(19) United States
(12) Patent Application Publication
(10) Pub. No.: US 2026/0035376 A1
(43) Pub. Date: Feb. 5, 2026

(54) COMPOSITIONS AND METHODS FOR INHIBITION OF RAS

(71) Applicants: TheRas, Inc., South San Francisco, CA (US); Leidos Biomedical Research, Inc., Frederick, MD (US); Lawrence Livermore National Security, LLC, Livermore, CA (US)

(72) Inventors: Zuhui Zhang, Millbrae, CA (US); Bin Wang, Dallas, TX (US); Rui Xu, Dallas, TX (US); Eli Wallace, Loveland, CO (US); Yue Yang, Foster City, CA (US); Felice Lightstone, Fremont, CA (US); Jun Pei, Livermore, CA (US); Anna Elzbieta Maciag, Frederick, MD (US); David Michael Turner, Frederick, MD (US); Dhirendra Kumar Simanshu, Frederick, MD (US); Albert Hay Wah Chan, Frederick, MD (US); Christopher John Brassard, Frederick, MD (US); Tao Liao, Frederick, MD (US)

(21) Appl. No.: 19/100,848

(22) PCT Filed: Aug. 4, 2023
(86) PCT No.: PCT/US2023/029537

§ 371 Date: Feb. 3, 2025

Related U.S. Application Data
(60) Provisional application No. 63/395,700, filed on Aug. 5, 2022.

(51) Int. Cl. C07D 487/08; A61K 31/517; A61K 31/551; A61K 31/554; C07D 409/14; C07D 519/00

(52) U.S. Cl. CPC C07D 487/08; A61K 31/517; A61K 31/551; A61K 31/554; C07D 409/14; C07D 519/00

(57) ABSTRACT

Provided herein are compounds, or salts, esters, tautomers, prodrugs, zwitterionic forms, or stereoisomers thereof, as well as pharmaceutical compositions comprising the same. Also provided herein are methods of using the same in modulating (e g., inhibiting) KRAS (e.g., KRAS having a G12D mutation) and treating diseases or disorders such as cancers in subjects in need thereof.

### RELATED APPLICATIONS

This application claims priority to and benefit of U.S. Application No. 63/395,700, filed Aug. 5, 2022, the entire contents of which are hereby incorporated by reference.

### STATEMENT AS TO RIGHTS TO INVENTIONS MADE UNDER FEDERALLY SPONSORED RESEARCH AND DEVELOPMENT

This invention was made with government support under (1) Contract No.: 75N91019D00024 awarded by the National Institutes of Health and (2) Contract No. DE-AC52-07NA27344 awarded by the United States Department of Energy. The government has certain rights in the invention.

### BACKGROUND

RAS mutations occur in approximately 20-30% of human cancers, including the majority of pancreatic ductal adenocarcinoma (PDAC), half of colorectal cancers, and a third of all lung cancers. With the highest RAS mutation frequencies seen with the top three causes of cancer deaths in the United States (lung, colorectal, and pancreatic cancer), the development of anti-RAS therapies is a major priority and a major challenge for cancer research. RAS proteins did not appear to present suitable pockets to which drugs could bind, except for the GDP/GTP binding site. Unfortunately, RAS proteins bind to these nucleotides with very high (picomolar) affinities, making the development of effective nucleotide analogs virtually impossible. Attempts to block pathways downstream of RAS with a hope to provide clinical benefit for patients suffering from RAS-driven cancers have been generally disappointing. Very recently, approved allele-specific covalent KRAS G12C inhibitors that bind and sequester the oncogenic protein in its inactive, GDP-bound state, have demonstrated clinical efficacy in patients with KRAS G12C cancers, including NSCLC, CRC and pancreatic adenocarcinoma.

The three RAS genes (HRAS, NRAS, and KRAS) encode four 188-189 amino acid proteins that share 82%-90% amino acid sequence identity and near-identical structural and biochemical properties. However, they are differentially expressed, and mutated with different frequencies in cancer. KRAS is the most frequently mutated oncogene in cancer and KRAS mutation is commonly associated with poor prognosis and resistance to therapy. Significant cancer type preferences exist among the RAS genes. KRAS mutations predominate in lung, colorectal, and pancreatic cancer, whereas NRAS mutations predominate in cutaneous melanomas and acute myelogenous leukemia, and HRAS mutations are found in bladder and head and neck squamous cell carcinomas.

KRAS is mutationally activated in 94% of pancreatic cancers. Pancreatic cancer (e.g., pancreatic ductal adenocarcinoma (PDAC)) became the third leading cause of cancer deaths in the United States in 2016. With a continued increase in incidence, pancreatic cancer is projected to become the second leading cause of cancer death by 2020. With no biomarkers for early detection, late onset of symptoms when the cancer has already reached the metastatic state, and the 5-year survival rate of 8%, pancreatic cancer is the deadliest cancer in the US.

KRAS mutations are the initiating genetic step in pancreatic cancer; however, continued mutant KRAS function is required to maintain the growth of PDAC. RNA interference-mediated KRAS inactivation in KRAS G12D-driven PDAC showed rapid regression of tumor growth. These data support the significance of mutant KRAS as a therapeutic target in PDAC. Since 40% of PDAC are driven by KRAS G12D mutant, inhibitors targeting this mutation are highly desirable. KRAS G12D mutation also occurs in high frequency in lung and colorectal cancers, making KRAS G12D desirable therapeutic target for direct G12D allele-specific inhibitors.

### SUMMARY

The present disclosure provides compounds, as well as compositions and kits comprising the same, and methods of using the same in the treatment of diseases and disorders such as cancers. The present disclosure provides compounds that may be capable of inhibiting one or more mutant forms of KRAS, such as KRAS having a G12D, G12V, G12C, G12S, G12A, G12R, Q61H, or G13D mutation, or wild-type KRAS. In particular, such compounds may be capable of selectively inhibiting a KRAS protein having a G12D mutation. In some embodiments, the compounds provided herein may be capable of targeting both active GTP-bound protein and inactive GDP-bound protein, which inhibitors may provide therapeutic advantages over compounds capable of targeting only the inactive GDP-bound protein. In some embodiments, compounds provided herein have inhibitory activity against a KRAS protein comprising a glycine to aspartic acid, valine, cysteine, serine, alanine, or arginine mutation at codon 12 (i.e., a G12D, G12V, G12C, G12S, G12A, or G12R mutation); or a glycine to aspartic acid mutation at codon 13 (e.g., a G13D mutation); or a glutamine to histidine mutation at codon 61 (e.g., a Q61H mutation) in both its active and inactive conformations; in particular embodiments, compounds provided herein have inhibitory activity against a KRAS protein comprising a glycine to aspartic acid mutation at codon 12 (i.e., a G12D mutation). In some embodiments, compounds provided herein are useful in the treatment of cancers, such as cancers characterized by KRAS proteins having a G12D mutation.

In an aspect, the present disclosure provides compositions comprising compounds represented by Formula I.

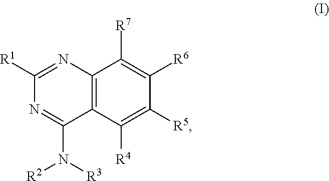

or a salt (e.g., pharmaceutically acceptable salt), ester, tautomer, prodrug, zwitterionic form, or stereoisomer thereof, wherein R¹, R², R³, R⁴, R⁵, R⁶, and R⁷ are as provided herein. In some embodiments, the compound is a compound according to any one of Formulas I, IA, IB, IC, ID, IE, IF, IG, IH, IJ, IK, IL, IM, and IN provided herein, or a salt (e.g., pharmaceutically acceptable salt), ester, tautomer, prodrug, zwitterionic form, or stereoisomer thereof. In some embodiments, a compound provided herein, or a salt, ester, tautomer, zwitterionic form, or stereoisomer thereof, can modulate (e.g., inhibit) the activity of a KRAS protein, such as a KRAS protein having a mutation at a G12D mutation. In some embodiments, a compound provided herein, or a salt, ester, tautomer, zwitterionic form, or stereoisomer thereof, is capable of interacting with a KRAS protein comprising a G12D mutation in both its active and inactive conformations. In some embodiments, a compound provided herein, or a salt, ester, tautomer, zwitterionic form, or stereoisomer thereof, is capable of binding a KRAS protein in an active (“GTP-bound”) conformation. In some embodiments, a compound provided herein, or a salt, ester, tautomer, zwitterionic form, or stereoisomer thereof, is capable of binding a KRAS protein in an inactive (“GDP-bound”) conformation. In some embodiments, a compound provided herein, or a salt, ester, tautomer, zwitterionic form, or stereoisomer thereof, is capable of binding a KRAS protein in both its active (“GTP-bound”) and inactive (“GDP-bound”) conformations.

In another aspect, the present disclosure provides a pharmaceutical composition comprising a compound provided herein (e.g., a compound represented by one of Formulas I, IA, IB, IC, ID, IE, IF, IG, IH, IJ, IK, IL, IM, and IN, or any other formula set forth herein), or a salt, ester, tautomer, prodrug, zwitterionic form, or stereoisomer thereof together with a pharmaceutically acceptable carrier.

In a further aspect, the present disclosure provides a method of inhibition of KRAS activity in a human or animal subject for the treatment of a disease such as cancer, including pancreatic cancer (e.g., pancreatic ductal adenocarcinoma (PDAC)), colorectal cancer, endometrial endometrioid adenocarcinoma, rectal adenocarcinoma, gastric cancer, and lung cancer, using, e.g., a compound provided herein (e.g., a compound represented by one of Formulas I, IA, IB, IC, ID, IE, IF, IG, IH, IJ, IK, IL, IM, and IN, or any other formula set forth herein), or a salt, ester, tautomer, prodrug, zwitterionic form, or stereoisomer thereof, or a pharmaceutical composition comprising the same.

In another aspect, the present disclosure provides a use of a compound provided herein (e.g., a compound represented by one of Formulas I, IA, IB, IC, ID, IE, IF, IG, IH, IJ, IK, IL, IM, and IN, or any other formula set forth herein), or a salt, ester, tautomer, zwitterionic form, or stereoisomer thereof, in the manufacture of a medicament for the treatment of a disease, disorder, or condition (e.g., a cancer) ameliorated, treated, inhibited, or reduced by inhibition of KRAS, including KRAS having a mutation at codon 12 (e.g., a G12D mutation). In some embodiments, the disease, disorder, or condition is pancreatic cancer (e.g., pancreatic ductal adenocarcinoma (PDAC)), colorectal cancer, or lung cancer.

In a further aspect, the present disclosure provides a compound as provided herein (e.g., a compound represented by one of Formulas I, IA, IB, IC, ID, IE, IF, IG, IH, IJ, IK, IL, IM, and IN, or any other formula set forth herein), or a salt, ester, tautomer, zwitterionic form, or stereoisomer thereof, for use as a medicament. In some embodiments, the medicament is used in the treatment of a disease, disorder, or condition (e.g., a cancer). In some embodiments, the disease, disorder, or condition is pancreatic cancer (e.g., pancreatic ductal adenocarcinoma (PDAC)), colorectal cancer, or lung cancer.

### DETAILED DESCRIPTION

The present disclosure provides compounds (e.g., compounds of Formulas I, IA, IB, IC, ID, IE, IF, IG, IH, IJ, IK, IL, IM, and IN), which compounds may possess useful KRAS inhibitory activity, and may be used in the treatment or prophylaxis of a disease, disorder, or condition in which KRAS plays an active role. In particular, certain compounds provided herein may possess useful inhibitory activity of a KRAS protein having a mutation at codon 12 (e.g., a G12D mutation), which KRAS protein is in an active (GTP-bound) or inactive (GDP-bound) conformation. Certain compounds provided herein may be capable of inhibiting both active and inactive forms of KRAS. The present disclosure also provides pharmaceutical compositions comprising one or more compounds provided herein together with a pharmaceutically acceptable carrier, as well as methods of making and using the compounds and compositions. The present disclosure also provides methods for inhibiting KRAS, including a KRAS protein having a mutation at codon 12 (e.g., a G12D mutation), which KRAS is in an active or inactive conformation. In an aspect, the present disclosure provides a method for treating a disorder mediated by KRAS including a KRAS protein having a mutation at codon 12 (e.g., a G12D mutation) in a subject in need of such treatment, which method comprises administering to the subject a therapeutically effective amount of a compound or composition provided herein. Also provided herein is the use of certain compounds provided herein in the manufacture of a medicament for the treatment of a disease, disorder, or condition ameliorated, treated, inhibited, or reduced by inhibition of KRAS, including a KRAS protein having a mutation at codon 12 (e.g., a G12D mutation). In some embodiments, the disease, disorder, or condition is a cancer (e.g., as described herein).

When ranges of values are disclosed, and the notation “from n₁ . . . to n₂” or “between n₁ . . . and n₂” is used, where n i and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Acyl,” as used herein, alone or in combination, refers to a carbonyl attached to an alkenyl, alkyl, aryl, cycloalkyl, heteroaryl, heterocycle, or any other moiety where the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

“Alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

“Alkynyl” refers to either a straight chain or branched-chain hydrocarbon having at least 2 carbon atoms and at least one triple bond and having the number of carbon atoms indicated (i.e., C₂₋₆ means to two to six carbons). Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, and 1,3,5-hexatriynyl.

“Alkoxy,” as used herein, alone or in combination, refers to an alkyl ether radical, wherein the term alkyl is as described herein. Examples of suitable alkyl ether radicals include methoxy, ethoxy, n-propoxy, isopropoxy, n-butoxy, iso-butoxy, sec-butoxy, tert-butoxy, and the like.

“Alkyl,” as used herein, alone or in combination, refers to a straight-chain or branched-chain alkyl radical containing from 1 to 20 carbon atoms (e.g., C₁₋₂₀ alkyl). In certain embodiments, said alkyl will comprise from 1 to 10 carbon atoms (e.g., C₁₋₁₀ alkyl). In further embodiments, said alkyl will comprise from 1 to 8 carbon atoms (e.g., C₁₋₈ alkyl). In further embodiments, said alkyl will comprise from 1 to 6 carbon atoms (e.g., C₁₋₆ alkyl). In further embodiments, said alkyl will comprise from 1 to 3 carbon atoms (e.g., C₁₋₃ alkyl). Alkyl groups are unsubstituted or substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl, and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

“Alkylamino,” as used herein, alone or in combination, refers to an alkyl group attached to the parent molecular moiety through an amino group. Suitable alkylamino groups maybe mono- or dialkylated, forming groups such as, for example, N-methylamino, N-ethylamino, N,N-dimethylamino, N,N-ethylmethylamino, and the like.

“Alkylthio,” as used herein, alone or in combination, refers to an alkyl thioether (R—S—) radical wherein the term alkyl is as described herein and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

“Amido” and “carbamoyl,” as used herein, alone or in combination, refer to an amino group as described herein attached to the parent molecular moiety through a carbonyl group, or vice versa. The “amido” group as used herein incudes a “C-amido” and “N-amido” groups. The term “C-amido” as used herein, alone or in combination, refers to a —C(O)N(RR′) group with R and R′ as defined herein or as defined by the specifically enumerated “R” groups designated. In some embodiments, the “amido” group includes —C(O)NH₂, C₁₋₄alkylamido, and di(C₁₋₄alkyl)amido. The term “C₁₋₄alkylamido”, as used herein, refers to —C(O)NH(C₁₋₄alkyl), wherein C₁₋₄alkyl is as defined herein. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

“Amino,” as used herein, alone or in combination, refers to —NRR′, wherein R and R′ are independently selected from hydrogen, alkyl, acyl, heteroalkyl, aryl, cycloalkyl, heteroaryl, and heterocycloalkyl, any of which may themselves be unsubstituted or substituted. Additionally, R and R′ may combine to form a heterocycloalkyl, which is unsubstituted or substituted. An “amino” group may be a primary amine (e.g., —NH₂), secondary or di-substituted amine (e.g., —NHR where R is not hydrogen), or tertiary or tri-substituted amine (e.g., —NRR′ where neither R nor R′ is hydrogen).

“Aryl,” as used herein, alone or in combination, means a carbocyclic aromatic system containing one, two, or three rings wherein such polycyclic ring systems are fused together. The term “aryl” embraces aromatic groups such as phenyl, naphthyl, anthracenyl, and phenanthryl. An aryl moiety may include, for example, between 5 to 20 carbon atoms, such as between 5 to 12 carbon atoms, such as 5 or 6 carbon atoms.

“Arylalkenyl” or “aralkenyl,” as used herein, alone or in combination, refers to an aryl group attached to the parent molecular moiety through an alkenyl group.

“Arylalkoxy” or “aralkoxy,” as used herein, alone or in combination, refers to an aryl group attached to the parent molecular moiety through an alkoxy group.

“Arylalkyl” or “aralkyl,” as used herein, alone or in combination, refers to an aryl group attached to the parent molecular moiety through an alkyl group.

“Aryloxy,” as used herein, alone or in combination, refers to an aryl group attached to the parent molecular moiety through an oxy.

“Carbamate,” as used herein, alone or in combination, refers to an ester of carbamic acid (—NHCOO—) which may be attached to the parent molecular moiety from either the nitrogen or acid end, and which is unsubstituted or substituted as defined herein.

“O-carbamyl” as used herein, alone or in combination, refers to a —OC(O)NRR′ group, with R and R′ as defined herein.

“N-carbamyl” as used herein, alone or in combination, refers to a ROC(O)NR′— group, with R and R′ as defined herein.

“Carbonyl,” as used herein, when alone includes formyl [—C(O)H] and in combination is a —C(O)— group.

“Carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

“Cyano,” as used herein, alone or in combination, refers to —CN.

“Cycloalkyl,” or, alternatively, “carbocycle,” as used herein, alone or in combination, refers to a saturated or partially saturated monocyclic, bicyclic, or tricyclic alkyl group wherein each cyclic moiety contains from 3 to 12 carbon atom ring members and which may optionally be a benzo fused ring system which is unsubstituted or substituted as defined herein. A carbocycle may comprise a bridged ring system and/or a spiro ring system (e.g., a system including two rings sharing a single carbon atom). The term “cycloalkenyl” refers to a cycloalkyl group having one or two double bonds. In certain embodiments, said cycloalkyl (or cycloalkenyl) will comprise from 5 to 7 carbon atoms. Examples of such groups include cyclopropyl, cyclobutyl, cyclopentyl, cyclohexyl, cycloheptyl, cyclobutenyl, cyclopentenyl, cyclohexenyl, tetrahydronapthyl, indanyl, octahydronaphthyl, 2,3-dihydro-IH-indenyl, adamantyl, and the like. “Bicyclic” and “tricyclic” as used herein are intended to include both fused ring systems, such as decahydronaphthalene and octahydronaphthalene, as well as the multicyclic (multicentered) saturated or partially unsaturated type. The latter type of isomer is exemplified in general by bicyclo[1,1,1]pentane, camphor, adamantane, and bicyclo[3,2,1]octane.

“Ester,” as used herein, alone or in combination, refers to a carboxy group bridging two moieties linked at carbon atoms.

“Ether,” as used herein, alone or in combination, refers to an oxy group bridging two moieties linked at carbon atoms.

“Halo,” or “halogen,” as used herein, alone or in combination, refers to fluorine, chlorine, bromine, or iodine.

“Haloalkoxy,” as used herein, alone or in combination, refers to a haloalkyl group attached to the parent molecular moiety through an oxygen atom.

“Haloalkyl,” as used herein, alone or in combination, refers to an alkyl radical having the meaning as described herein wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro, or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

“Heteroalkyl,” as used herein, alone or in combination, refers to a stable straight or branched hydrocarbon chain, fully saturated or containing from 1 to 3 degrees of unsaturation, consisting of the stated number of carbon atoms and from one to three heteroatoms selected from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

“Heteroaryl,” as used herein, alone or in combination, refers to a 3- to 15-membered aromatic monocyclic ring, or a fused monocyclic, bicyclic, or tricyclic ring system in which at least one of the fused rings is aromatic, which ring or ring system contains at least one atom selected from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, phenanthrolinyl, dibenzofuranyl, acridinyl, phenanthridinyl, xanthenyl and the like.

“Heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. A heterocycle may comprise a bridged ring system and/or a spiro ring system (e.g., a system including two rings sharing a single atom, such as a single carbon atom). “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups are unsubstituted or substituted unless specifically prohibited.

“Hydrazinyl” as used herein, alone or in combination, refers to two amino groups joined by a single bond, i.e., —N—N—.

“Hydroxy,” as used herein, alone or in combination, refers to —OH.

“Hydroxyalkyl,” as used herein, alone or in combination, refers to a hydroxy group attached to the parent molecular moiety through an alkyl group.

“Iminohydroxy,” as used herein, alone or in combination, refers to ═N(OH) and ═N—O—.

“Lower amino,” as used herein, alone or in combination, refers to —NRR′, wherein R and R′ are independently selected from hydrogen and lower alkyl (e.g., C₁₋₄ alkyl), either of which is unsubstituted or substituted.

“Mercaptyl” as used herein, alone or in combination, refers to an RS— group, where R is as defined herein.

“Nitro,” as used herein, alone or in combination, refers to —NO₂.

“Oxy” or “oxa,” as used herein, alone or in combination, refer to —O—.

“Oxo,” as used herein, alone or in combination, refers to ═O.

“Perhaloalkoxy” refers to an alkoxy group where all of the hydrogen atoms are replaced by halogen atoms.

“Perhaloalkyl” as used herein, alone or in combination, refers to an alkyl group where all of the hydrogen atoms are replaced by halogen atoms.

“Ring,” or equivalently, “cycle,” as used herein, in reference to a chemical structure or portion thereof, means a group in which every atom is a member of a common cyclic structure. A ring can be saturated or unsaturated, including aromatic, unless otherwise provided, and may have between 3 and 9 members. If the ring is a heterocycle, it may contain between 1 and 4 heteroatoms or heteroatom-comprising groups selected from B, N, O, S, C(O), and S(O)ₘ, wherein m is 0, 1, or 2. Unless specifically prohibited, a ring is unsubstituted or substituted. Two or more rings may be fused together (e.g., they may share a bond and two common atoms). Two or more rings may be linked together in a spiro arrangement such that only a single atom is shared between two rings. Two or more rings may also or alternatively be configured in a bridged arrangement such that three or more atoms are shared between two or more rings.

“Sulfonate,” “sulfonic acid,” and “sulfonic,” as used herein, alone or in combination, refer to the —SO₃H group and its anion as the sulfonic acid is used in salt formation.

“Sulfanyl,” as used herein, alone or in combination, refers to —S—.

“Sulfinyl,” as used herein, alone or in combination, refers to —S(O)—.

“Sulfonyl,” as used herein, alone or in combination, refers to —S(O)₂—.

“N-sulfonamido” refers to a RS(═O)₂NR′— group with R and R′ as defined herein.

“S-sulfonamido” refers to a —S(═O)₂NRR′, group, with R and R′ as defined herein.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers that are well known in the art include keto/enol, enamine/imine, and lactam/lactim tautomers. Examples of tautomers that are well known in the art also include 2-hydroxypyridine/2(1H)-pyridone and 2-aminopyridine/2(1H)-iminopyridone tautomers.

“Thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

“Thiol,” as used herein, alone or in combination, refers to an —SH group.

“Thiocarbonyl,” as used herein, when alone includes thioformyl —C(S)H and in combination is a —C(S)— group.

“N-thiocarbamyl” refers to an ROC(S)NR′— group, with R and R′ as defined herein.

“O-thiocarbamyl” refers to a —OC(S)NRR′ group, with R and R′ as defined herein.

“Thiocyanato” refers to a —CNS group.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

As described herein, groups may be substituted or unsubstituted (e.g., “optionally substituted”). Unless otherwise specified, any group may be substituted with one or more substituents, such as one or more substituents provided herein. Examples of substituents that may substitute a group include, but are not limited to, one or more substituents independently selected from the following groups or a particular designated set of groups, alone or in combination: alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), alkynyl (e.g., C₂₋₂₀ alkynyl, such as C₂₋₁₀ alkynyl, such as C₂₋₆ alkynyl), alkanoyl (e.g., C₁₋₂₀ alkanoyl, such as C₁₋₁₀ alkanoyl, such as C₁₋₆ alkanoyl), heteroalkyl (e.g., a heteroalkyl moiety including 1-20 carbon atoms and 1-6 heteroatoms, such as a heteroalkyl moiety including 1-6 carbon atoms and 1-3 heteroatoms), haloalkyl (e.g., a halo-substituted C₁₋₂₀ alkyl, such as a halo-substituted C₁₋₁₀ alkyl, a halo-substituted C₁₋₆ alkyl), haloalkenyl (e.g., a halo-substituted C₂₋₂₀ alkenyl, such as a halo-substituted C₂₋₆ alkenyl), haloalkynyl (e.g., a halo-substituted C₂₋₂₀ alkynyl, such as a halo-substituted C₂₋₆ alkynyl), perhaloalkyl (e.g., C₁₋₂₀ perhaloalkyl, such as C₁₋₆ perhaloalkyl, such as C₁₋₃ perhaloalkyl), perhaloalkoxy (e.g., C₁₋₂₀ perhaloalkoxy, such as C₁₋₆ perhaloalkoxy), phenyl, aryl (e.g., C₅₋₂₀ aryl, such as C₅₋₁₀ aryl, such as C₅₋₆ aryl), aryloxy (e.g., C₅₋₂₀ aryloxy, such as C₅₋₁₀ aryloxy, such as C₅₋₆ aryloxy), alkoxy (e.g., C₁₋₂₀ alkoxy, such as C₁₋₁₀ alkoxy, such as C₁₋₆ alkoxy), haloalkoxy (e.g., C₁₋₂₀ haloalkoxy, such as C₁₋₁₀ haloalkoxy, such as C₁₋₆ haloalkoxy), oxo, acyloxy (e.g., an acyloxy group including 1-20 carbon atoms, such as 1-10 carbon atoms, such as 1-6 carbon atoms), carbonyl (e.g., C(O) or C═O), carboxyl (e.g., C(O)O), alkylcarbonyl (e.g., C₁₋₂₀ alkylcarbonyl, such as C₁₋₁₀ alkylcarbonyl, such as C₁₋₆ alkylcarbonyl, such as C₁₋₃ alkylcarbonyl), carboxyester (e.g., C(O)OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), carboxamido, cyano (e.g., CN), hydrogen, halogen (e.g., iodine, bromine, chlorine, or fluorine), hydroxy, amino (e.g., NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), alkylamino (e.g., NR′R″ where R′ is alkyl (e.g., C₁₋₂₀ alkyl, such as C₁₋₁₀ alkyl, such as C₁₋₆ alkyl, such as C₁₋₃ alkyl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), arylamino (e.g., NR′R″ where R′ is aryl (e.g., C₅₋₂₀ aryl, such as C₅₋₁₀ aryl, such as C₅₋₆ aryl) and R″ is, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), amido (e.g., C(O)NR′R″ where R′ and R″ are independently, e.g., hydrogen, alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), nitro (e.g., NO₂), thiol (e.g., SH), alkylthio (e.g., C₁₋₂₀ alkyl substituted with a thiol group, such as C₁₋₁₀ alkyl substituted with a thiol group, such as C₁₋₆ alkyl substituted with a thiol group, such as C₁₋₃ alkyl substituted with a thiol group), haloalkylthio (e.g., C₁₋₂₀ haloalkylthio, such as C₁₋₁₀ haloalkylthio, such as C₁₋₆ haloalkylthio, such as C₁₋₃ haloalkylthio), perhaloalkylthio (e.g., C₁₋₂₀ perhaloalkylthio, such as C₁₋₁₀ perhaloalkylthio, such as C₁₋₆ perhaloalkylthio, such as C₁₋₃ perhaloalkylthio), arylthiol (e.g., C₅₋₂₀ arylthiol, such as C₅₋₁₀ arylthiol, such as C₅₋₆ arylthiol), sulfonate (e.g., S(O)₂OR where R is, e.g., alkyl (e.g., C₁₋₂₀ alkyl, such as C₁₋₁₀ alkyl, such as C₁₋₆ alkyl, such as C₁₋₃ alkyl), alkenyl (e.g., (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein), sulfonic acid (e.g., S(O)₂OH), trisubstituted silyl (e.g., SiR′R″R* where R′, R″, and R* are independently selected from, e.g., alkyl (e.g., C₁₋₂₀ alkyl, such as C₁₋₁₀ alkyl, such as C₁₋₆ alkyl, such as C₁₋₃ alkyl), alkenyl (e.g., C₂₋₂₀ alkenyl, such as C₂₋₁₀ alkenyl, such as C₂₋₆ alkenyl), or alkynyl (e.g., C₂₋₂₀ alkynyl, such as C₂₋₁₀ alkynyl, such as C₂₋₆ alkynyl), any of which may be substituted by any group provided herein; in some cases, a trisubstituted silyl can be trimethylsilyl), N₃, SCH₃, C(O)CH₃, CO₂CH₃, CO₂H, pyridinyl, thiophene, furanyl, carbamate, and urea. Additional groups may also be contemplated. Where structurally feasible, two substituents may be joined together to form a fused five-, six-, or seven-membered carbocyclic or heterocyclic ring consisting of zero to three heteroatoms (e.g., N, O, S, etc.), for example forming methylenedioxy or ethylenedioxy. An unsubstituted or substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “unsubstituted or substituted with.”

The terms R, R′, R″, R*, etc., appearing by themselves and without a number designation, unless otherwise defined, refer to a moiety selected from hydrogen, alkyl, cycloalkyl, heteroalkyl, aryl, heteroaryl and heterocycloalkyl, any of which is unsubstituted or substituted (e.g., as described herein). Such R and R′ groups should be understood to be unsubstituted or substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g., aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

“Bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

Asymmetric centers may exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, atropisomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds provided herein may comprise conformational isomers, which compounds comprise groups that can orient in different conformations in relation to another moiety. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Combination therapy” means the administration of two or more therapeutic agents to treat a therapeutic condition or disorder described in the present disclosure. Such administration encompasses co-administration of these therapeutic agents in a substantially simultaneous manner, such as in a single dose unit (e.g., capsule) having a fixed ratio of active ingredients or in multiple, separate dose units (e.g., capsules) for each active ingredient. In addition, such administration also encompasses use of each type of therapeutic agent in a sequential manner. In either case, the treatment regimen will provide beneficial effects of the drug combination in treating the conditions or disorders described herein.

“KRAS inhibitor” is used herein to refer to a compound that exhibits an IC₅₀ with respect to KRAS activity of no more than about 100 μM and more typically not more than about 50 μM, as measured in the assays described generally herein, such as a surface plasmon resonance KRAS-G12D, G12V, G12C, G12S, G12A, G12R, G13D, or Q61H mutation or wild-type KRAS protein binding assay; and/or a KRAS G12D, G12V, G12C, G12S, G12A, G12R, G13D, or Q61H mutation or wild-type KRAS protein-effector protein interaction disruption assay. “IC₅₀” is that concentration of inhibitor which reduces the activity of an enzyme (e.g., KRAS) to half-maximal level. Certain compounds disclosed herein have been discovered to exhibit inhibition against KRAS. In certain embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of no more than about 50 μM; in further embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of no more than about 10 M; in yet further embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of not more than about 1 μM; in yet further embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of not more than about 200 nanomolar (nM), as measured in the KRAS assay described herein. In some embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In certain embodiments, compounds exhibit an IC₅₀ with respect to KRAS (e.g., a KRAS protein having a mutation at codon 12 (e.g., a G12D, G12V, G12C, G12S, G12A, or G12R mutation), a KRAS protein having a mutation at codon 13 (e.g., a G13D mutation), a KRAS protein having a mutation at codon 61 (e.g., a Q61H mutation), or a wild-type KRAS protein) of less than about 1 μM, such as less than about 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less. In some embodiments, compounds exhibit an IC₅₀ with respect to KRAS having a G12D mutation of less than about 50 μM, such as less than about 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less.

In some embodiments, a KRAS inhibitor has inhibitory activity against KRAS having a G12D mutation that exceeds its inhibitory activity against KRAS having another mutation, such as a Q61H, G12C, G12R, G12S, G12A, G12V, or G13D mutation. In some embodiments, a KRAS inhibitor provided herein has greater inhibitory activity against KRAS having a G12D mutation than against KRAS having a G12C mutation. In some embodiments, a KRAS inhibitor provided herein has greater inhibitory activity against KRAS having a G12D mutation than against KRAS having a G12R mutation. In some embodiments, a KRAS inhibitor provided herein has greater inhibitory activity against KRAS having a G12D mutation than against a KRAS having a G12S mutation. In some embodiments, a KRAS inhibitor provided herein has greater inhibitory activity against KRAS having a G12D mutation than against KRAS having a G12A mutation. In some embodiments, a KRAS inhibitor provided herein has greater inhibitory activity against KRAS having a G12D mutation than against KRAS having a G12V mutation. In some embodiments, a KRAS inhibitor provided herein has greater inhibitory activity against KRAS having a G12D mutation than against KRAS having a G13D mutation. In some embodiments, a KRAS inhibitor provided herein has greater inhibitory activity against KRAS having a G12D mutation than against KRAS having a Q61H mutation.

In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having another mutation such as a Q61H, G12C, G12R, G12S, G12A, G12V, or G13D mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a Q61H mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a G12C mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a G12R mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a G12S mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a G12A mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a G12V mutation. In some embodiments, a KRAS inhibitor provided herein has at least two-fold, five-fold, ten-fold, twenty-fold, thirty-fold, forty-fold, fifty-fold, one hundred-fold, or higher inhibitory activity against KRAS having a G12D mutation relative to KRAS having a G13D mutation.

In some embodiments, a KRAS inhibitor provided herein has greater inhibitory activity against active (“GTP-bound”) KRAS having a G12D mutation than against an inactive (“GDP-bound”) KRAS having a G12D mutation. In some embodiments, a KRAS inhibitor provided herein has lower inhibitory activity against active (“GTP-bound”) KRAS having a G12D mutation than against an inactive (“GDP-bound”) KRAS having a G12D mutation. In some embodiments, a KRAS inhibitor provided herein has inhibitory activity against both active (“GTP-bound”) and inactive (“GDP-bound”) KRAS having a G12D mutation. In some embodiments, a KRAS inhibitor provided herein has similar inhibitory activity against active (“GTP-bound”) and inactive (“GDP-bound”) KRAS having a G12D mutation. In some embodiments, a KRAS inhibitor provided herein has inhibitory activity against a K-RAS4a splice variant. In some embodiments, a KRAS inhibitor provided herein has inhibitory activity against a K-RAS4b splice variant. In some embodiments, a KRAS inhibitor provided herein has inhibitory activity against both K-RAS4a and K-RAS4b splice variants.

“Therapeutically effective amount” refers to an amount of a compound or of a pharmaceutical composition useful for treating or ameliorating an identified disease, disorder, or condition, or for exhibiting a detectable therapeutic or inhibitory effect. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

“Treat,” “treating,” and “treatment” refer to any indicia of success in the treatment or amelioration of an injury, pathology, disease, disorder, or condition, including any objective or subjective parameter such as abatement; remission; diminishing of symptoms or making the injury, pathology, disease, disorder, or condition more tolerable to the patient; slowing in the rate of degeneration or decline; making the final point of degeneration less debilitating; and/or improving a patient's physical or mental well-being. The treatment or amelioration of symptoms can be based on objective or subjective parameters, including the results of a physical examination, neuropsychiatric exams, and/or a psychiatric evaluation. Treatment may also be preemptive in nature; i.e., it may include prevention of a disease, disorder, or condition, prevention of onset of one or more symptoms of a disease, disorder, or condition, and/or prevention of escalation of a disease, disorder, or condition. Prevention of a disease, disorder, or condition may involve complete protection from disease, and/or prevention of disease progression (e.g., to a later stage of the disease, disorder, or condition). For example, prevention of a disease may not mean complete foreclosure of any effect related to the diseases at any level, but instead may mean prevention of the symptoms of a disease, disorder, or condition to a clinically significant or detectable level.

“Patient” or “subject” refers to a living organism suffering from or prone to a disease, disorder, or condition that can be treated by administration of a compound or pharmaceutical composition as provided herein. Non-limiting examples include humans, rats, mice, rabbits, hamsters, guinea pigs, cats, dogs, non-human primates (e.g., monkeys), goats, pigs, sheep, cows, deer, horses, and other non-mammalian animals. Examples of mammals that can be treated by administration of a compound or pharmaceutical composition provided herein include, for example, rodents (e.g., rats, mice, squirrels, guinea pigs, hamsters, etc.), lagomorphs (e.g., rabbits, hares, etc.), primates (e.g., monkeys, apes, etc.), bovines (e.g., cattle), odd-toed ungulates (e.g., horses), even-toed ungulates (e.g., bovines such as cattle, ovine such as sheep, caprine such as goats, porcine such as pigs, etc.), and marsupials (e.g., kangaroo, wallaby, wallaroo, sugar glider, etc.). In some embodiments, the patient or subject is human. In some embodiments, the patient or subject is a companion animal such as a cat or dog. In some embodiments, the patient or subject is a farm animal such as a goat, sheep, cow, pig, or horse. In some embodiments, the patient or subject is an exotic animal such as a primate (e.g., monkey), marsupial (e.g., kangaroo, wallaby, wallaroo, sugar glider, etc.), or a non-domesticated or hybrid cat or dog.

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent, or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors, and colors. One of skill in the art will recognize that other pharmaceutical excipients are useful in the present disclosure.

The term “prodrug” refers to a compound that is made more active in vivo. Certain compounds disclosed herein may also exist as prodrugs. Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug.

The compounds disclosed herein can exist as therapeutically acceptable salts (also referred to herein as “pharmaceutically acceptable salts”). The present disclosure includes compounds provided herein in the form of salts, including acid addition salts. Suitable salts include those formed with both organic and inorganic acids. Such acid addition salts will normally be pharmaceutically acceptable. However, non-pharmaceutically acceptable salts may be of utility in the preparation and purification of the compound in question. Basic addition salts may also be formed and be pharmaceutically acceptable.

The terms “therapeutically acceptable salt” and “pharmaceutically acceptable salt” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

“KRAS positive cancer” refers to a cancer characterized by a KRAS mutation, such as a KRAS Q61H, G12C, G12D, G12R, G12A, G12S, G12V, or G13D mutation, and/orby amplified wild-type KRAS activity. In some embodiments, “KRAS positive cancer” refers to a cancer that may benefit from inhibition of KRAS, such as wild-type KRAS or KRAS having a Q61H, G12C, G12D, G12R, G12A, G12S, G12V, or G13D mutation.

“KRAS G12C-positive cancer” refers to a cancer characterized by a KRAS G12C mutation.

“KRAS G12D-positive cancer” refers to a cancer characterized by a KRAS G12D mutation.

“KRAS G12R-positive cancer” refers to a cancer characterized by a KRAS G12R mutation.

“KRAS G12V-positive cancer” refers to a cancer characterized by a KRAS G12V mutation.

“KRAS G12A-positive cancer” refers to a cancer characterized by a KRAS G12A mutation.

“KRAS G12S-positive cancer” refers to a cancer characterized by a KRAS G12S mutation.

“KRAS G13D-positive cancer” refers to a cancer characterized by a KRAS G13D mutation.

“KRAS Q61H-positive cancer” refers to a cancer characterized by a KRAS Q61H mutation.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a KRAS inhibitor, as defined herein, and an additional agent, which additional agent may be an agent configured to treat a disease, disorder, or condition or a symptom thereof. The effect can be, for example, slowing the symptomatic progression of a proliferative disease, such as cancer, particularly lung cancer, or symptoms thereof. Analogously, a “synergistically effective amount” refers to the amount needed to obtain a synergistic effect.

“A,” “an,” or “a(n)”, when used in reference to a group of substituents or “substituent group” herein, mean at least one. For example, where a compound is substituted with “an” alkyl or aryl, the compound is unsubstituted or substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### Compounds

In an aspect, the present disclosure provides a compound represented by Formula I:

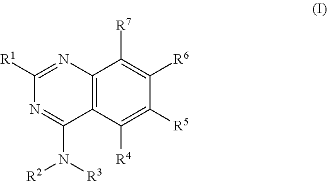

or a salt (e.g., pharmaceutically acceptable salt), ester, tautomer, prodrug, zwitterionic form, or stereoisomer thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is selected from H and C₁₋₆alkyl, wherein any C₁₋₆ alkyl is unsubstituted or is substituted with one or more R¹³
R³ is selected from a 4-6 membered heterocycle that is unsubstituted or substituted with one or more R¹⁰;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R³;
R⁶ is a bicyclic heteroaryl substituted with one or more R¹⁵;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
each R²⁰ is independently selected from —OH, —OC₁₋₆alkyl, —CN, —NH₂, —NHC₁₋₆alkyl, and halogen.

In some embodiments, the present disclosure provides a compound of Formula I, or a salt (e.g., a pharmaceutically acceptable salt) thereof.

In some embodiments, R⁶ is selected from:

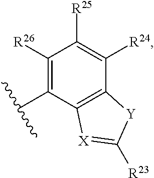

wherein X is selected from N and C—CN; Y is selected from O and S; R²³ is selected from —N(R¹²)₂, C₁₋₆alkyl, and C₁₋₆alkyl-N(R¹⁴)₂, wherein any C₁₋₆alkyl is unsubstituted or substituted with one or more R¹; and R²⁴, R²⁵, and R²⁶ are independently selected from H, halogen, and C₁₋₆alkyl, wherein any C₁₋₆alkyl is unsubstituted or substituted with one or more R¹³. In some embodiments, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂.

In some embodiments R⁶ is selected from:

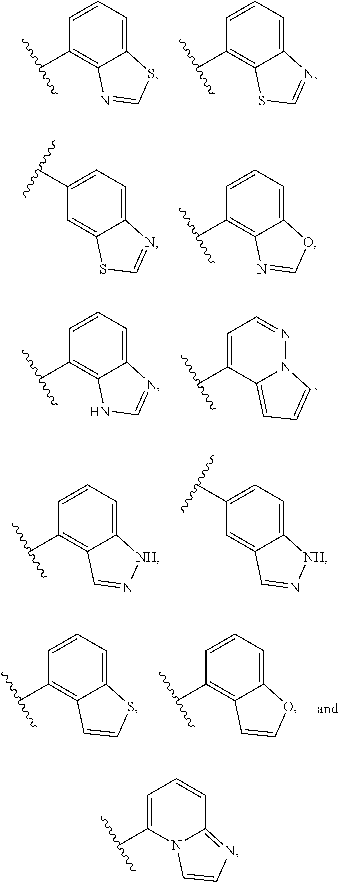

any of which is substituted with one or more R¹⁵.

In some embodiments, R⁶ is selected from:

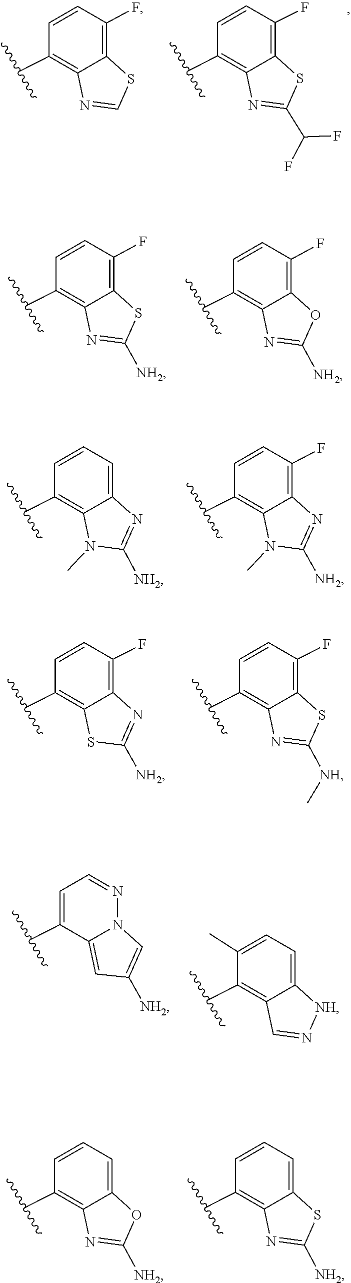

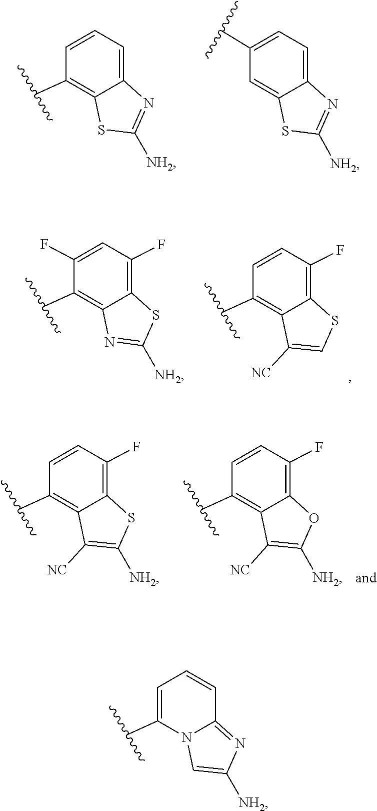

In some embodiments, R⁶ is selected from:

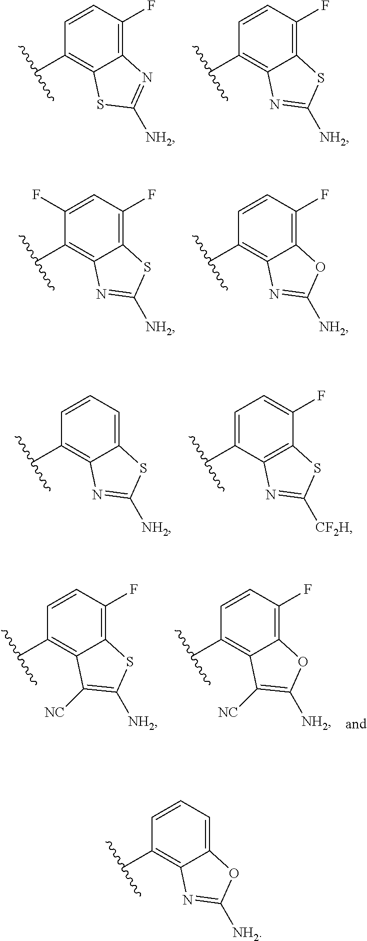

In some embodiments, R⁶ is

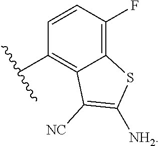 [SMILES: *c1ccc(F)c2sc(N)c(C#N)c12]

In some embodiments, the present disclosure provides a compound of Formula I:

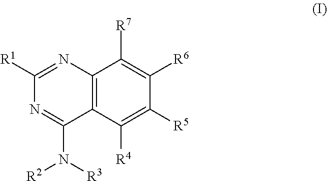

or a salt (e.g., pharmaceutically acceptable salt), ester, tautomer, prodrug, zwitterionic form, or stereoisomer thereof, wherein:
R¹ is selected from C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷,
provided that R¹ is not:

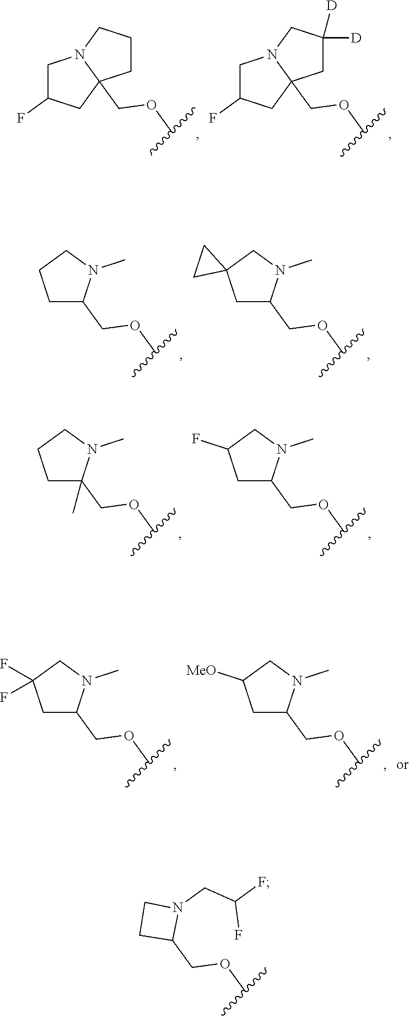

R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from H, halogen, —CN, —OR¹⁷, a 3-6 membered heterocycle, a 5-6 membered heteroaryl, and C₁₋₆alkyl, wherein any C₁₋₆alkyl is unsubstituted or substituted with one or more R¹³;
R⁶ is

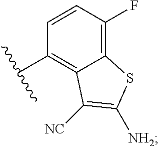 [SMILES: *c1ccc(F)c2sc(N)c(C#N)c12]

R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
each R²⁰ is independently selected from —OH, —OC₁₋₆alkyl, —CN, —NH₂, —NHC₁₋₆alkyl, and halogen.

In some embodiments, the present disclosure provides a compound of Formula I:

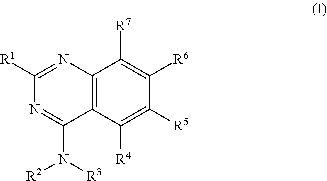

or a salt (e.g., pharmaceutically acceptable salt), ester, tautomer, prodrug, zwitterionic form, or stereoisomer thereof, wherein:
R¹ is selected from C₁₋₆alkyl, —OR⁸, —O(C₁₋₆alkylene)R⁸, —N(R¹⁶)₂, and a 4-, 5- or 7-membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen;
R⁸ is selected from C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
provided that R⁸ is not an unsubstituted or substituted heterocycle selected from:

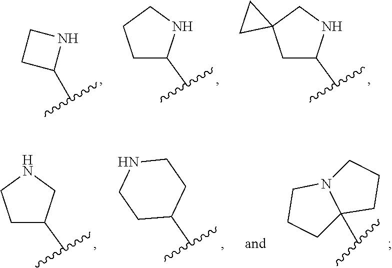 [SMILES: *C1CCN1 ; *C1CCCN1 ; *C1CC2(CC2)CN1 ; *C1CCNC1 ; *C1CCNCC1 ; *C12CCCN1CCC2]

each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
each R²⁰ is independently selected from —OH, —OC₁₋₆alkyl, —CN, —NH₂, —NHC₁₋₆alkyl, and halogen.

In some embodiments, the present disclosure provides a compound of Formula I:

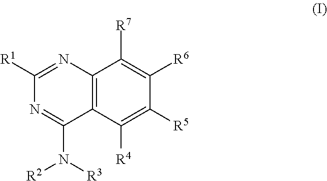

or a salt (e.g., pharmaceutically acceptable salt), ester, tautomer, prodrug, zwitterionic form, or stereoisomer thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is C₁₋₆alkyl;
R³ is a 4-6 membered heterocycle;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from halogen and C₁₋₆alkyl, wherein any C₁₋₆alkyl is unsubstituted or substituted with one or more R³;
R⁶ is a bicyclic heteroaryl substituted with one or more R¹⁵;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹¹ is independently C₁₋₆alkyl;
each R¹² is H;
each R¹³ is independently selected from —OR¹⁴, —CN, and halogen;
each R¹⁴ is independently selected from C₁₋₆ alkyl and H;
each R¹⁵ is independently selected from halogen, —N(R¹²)₂, and —CN;
each R¹⁶ is independently selected from H, C₁₋₆alkyl, —C(O)C₁₋₆alkyl, and —C(O)(C₀₋₆alkylene)heteroaryl, wherein any heteroaryl is unsubstituted or substituted with one or more R¹⁸;
each R¹⁷ is independently selected from —OR² and ═O;
each R¹⁸ is independently selected from C₁₋₆alkyl; and
each R¹⁹ is independently selected from —OR¹², ═O, and C₁₋₆alkyl.

In some embodiments, the compound is a compound according to Formula IA:

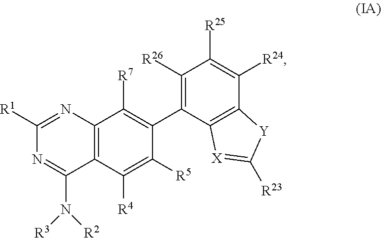

or a salt (e.g., pharmaceutically acceptable salt), ester, tautomer, prodrug, zwitterionic form, or stereoisomer thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is selected from H and C₁₋₆alkyl, wherein any C₁₋₆ alkyl is unsubstituted or is substituted with one or more R¹³
R³ is selected from a 4-6 membered heterocycle that is unsubstituted or substituted with one or more R¹⁰;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹¹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆alkenyl, and H;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R²⁰ is independently selected from —OH, —OC₁₋₆alkyl, —CN, —NH₂, —NHC₁₋₆alkyl, and halogen;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

In some embodiments, the present disclosure provides a compound of Formula IA, or a salt (e.g., a pharmaceutically acceptable salt) thereof.

In some embodiments, for a compound according to Formula IA, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R²)₂. In some embodiments, X is C—CN, Y is S, and R²³ is —NH₂. In some embodiments, X is C—CN, Y is S, R²³ is —N(R¹²)₂, and R²⁴ is a halogen (e.g., F). In some embodiments, X is C—CN, Y is S, R²³ is —N(R¹²)₂, and one or more of R²⁴, R²⁵, and R²⁶ is a halogen (e.g., F).

In some embodiments, for a compound according to Formula IA,

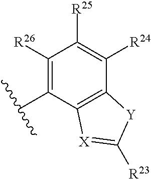

is

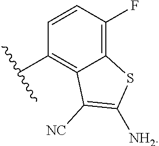 [SMILES: *c1ccc(F)c2sc(N)c(C#N)c12]

In some embodiments, for a compound according to Formula I or IA, R² is selected from H and C₁₋₆ alkyl, wherein any C₁₋₆ alkyl is unsubstituted or is substituted with one or more R¹³; and R³ is selected from a 4-6 membered heterocycle that is unsubstituted or substituted with one or more R¹⁰. In some embodiments, R³ is a 4-6 membered heterocycle that includes one or more heteroatoms selected from O, S, and N, wherein the heterocycle is unsubstituted or is substituted with one or more R¹⁰. In some embodiments, R³ is a 4-6 membered heterocycle that includes 1 heteroatom selected from O, S, and N, wherein the heterocycle is unsubstituted or is substituted with one or more R¹⁰. In some embodiments, R³ is a 4-6 membered heterocycle that includes 1 heteroatom selected from O, S, and N, wherein the heterocycle is substituted with 1-4 R¹⁰. In some embodiments, R³ is a 4-6 membered heterocycle that includes 1 heteroatom selected from O, S, and N, wherein the heterocycle is unsubstituted. In some embodiments, R³ is a 4-6 membered heterocycle that includes a nitrogen atom, wherein the heterocycle is substituted with 0-4 R¹⁰. In some embodiments, R³ is a 4-6 membered heterocycle that includes a nitrogen atom, wherein the heterocycle is unsubstituted. In some embodiments, R³ is an azetidine that is substituted with 0-4 R¹⁰. In some embodiments, R³ is an azetidine that is unsubstituted. In some embodiments, R³ is an azetidine that is substituted with 0-4 R¹⁰, provided that the azetidine includes an —NH— moiety. In some embodiments, R³ is a pyrrolidine that is substituted with 0-4 R¹⁰. In some embodiments, R³ is a pyrrolidine that is unsubstituted. In some embodiments, R³ is a pyrrolidine that is substituted with 0-4 RIU, provided that the pyrrolidine includes an —NH— moiety. In some embodiments, R³ is a piperidine that is substituted with 0-4 R¹⁰. In some embodiments, R³ is a piperidine that is unsubstituted. In some embodiments, R³ is a piperidine that is substituted with 0-4 R¹⁰, provided that the piperidine includes an —NH— moiety. In some embodiments, R³ is a 4-6 membered heterocycle that is substituted with one or more R¹⁰, wherein at least one R¹⁰ is selected from —OR¹² and a C₁₋₆alkyl substituted with —OH. In some embodiments, R³ is a 4-6 membered heterocycle that is substituted with one or more R¹⁰, wherein at least one R¹⁰ is an unsubstituted C₁₋₆alkyl. In some embodiments, R³ is selected from a 4-6 membered heterocycle that is substituted with one or more R¹⁰, wherein each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl that is unsubstituted or substituted with one or more R²⁰.

In some embodiments, the compound is a compound according to Formula IB, IC, ID, or IE:

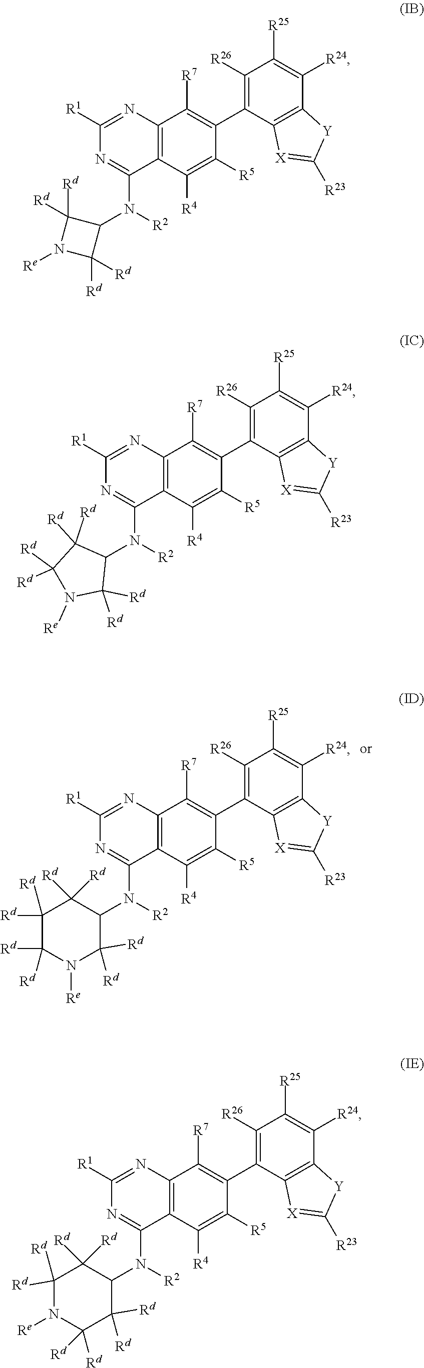

or a salt (e.g., pharmaceutically acceptable salt), ester, tautomer, prodrug, zwitterionic form, or stereoisomer thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is selected from H and C₁₋₆alkyl, wherein any C₁₋₆ alkyl is unsubstituted or is substituted with one or more R¹³;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R²⁰ is independently selected from —OH, —OC₁₋₆alkyl, —CN, —NH₂, —NHC₁₋₆alkyl, and halogen; each R^{d} is independently selected from H, —OR¹², ═O, —C(O)(C₁₋₆alkylene)CN, —C(O)(C₁₋₆alkylene)OH, —C(O)(C₁₋₆alkyl), —C(O)N(R¹⁴)₂, —S(O)₂(C₁₋₆alkyl), halogen, and C₁₋₆alkyl, wherein any C₁₋₆alkyl is unsubstituted or substituted with one or more R²⁰;
R^{e} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

In some embodiments, the compound is a compound according to Formula IB, or a salt (e.g., pharmaceutically acceptable salt), ester, tautomer, prodrug, zwitterionic form, or stereoisomer thereof. In some embodiments, the compound is a compound according to Formula IC, or a salt (e.g., pharmaceutically acceptable salt), ester, tautomer, prodrug, zwitterionic form, or stereoisomer thereof. In some embodiments, the compound is a compound according to Formula ID, or a salt (e.g., pharmaceutically acceptable salt), ester, tautomer, prodrug, zwitterionic form, or stereoisomer thereof. In some embodiments, the compound is a compound according to Formula IE, or a salt (e.g., pharmaceutically acceptable salt), ester, tautomer, prodrug, zwitterionic form, or stereoisomer thereof.

In some embodiments, the compound is a compound according to Formula IB, or a salt (e.g., pharmaceutically acceptable salt) thereof. In some embodiments, the compound is a compound according to Formula IC, or a salt (e.g., pharmaceutically acceptable salt) thereof. In some embodiments, the compound is a compound according to Formula ID, or a salt (e.g., pharmaceutically acceptable salt) thereof. In some embodiments, the compound is a compound according to Formula IE, or a salt (e.g., pharmaceutically acceptable salt) thereof.

In some embodiments, for a compound according to any one of Formulas IB, IC, ID, and IE, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S, and R²³ is —NH₂. In some embodiments, X is C—CN, Y is S, R²³ is —N(R²)₂, and R²⁴ is a halogen (e.g., F). In some embodiments, X is C—CN, Y is S, R²³ is —N(R¹²)₂, and one or more of R²⁴, R²⁵, and R²⁶ is a halogen (e.g., F).

In some embodiments, for a compound according to any one of Formulas IB, IC, ID, and IE,

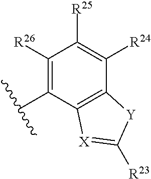

is

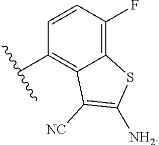 [SMILES: *c1ccc(F)c2sc(N)c(C#N)c12]

In some embodiments, for a compound of any one of Formulas IB, IC, ID, and IE, each R^{d} is H. In some embodiments, 0-2 R^{d} are C₁₋₆ alkyl and the remaining R^{d} groups are H. In some embodiments, at least one R^{d} is selected from —OR¹² and a C₁₋₆alkyl substituted with —OH. In some embodiments, each R^{d} is independently selected from H, —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl that is unsubstituted or substituted with one or more R²⁰. It will be appreciated that when R^{d} is =O, the other R^{d} on the same carbon atom is absent, such that the carbon has proper valency.

In some embodiments, for a compound according to any one of Formulas IB, IC, ID, and IE, R^{e} is H. In some embodiments, R^{e} is C₁₋₆alkyl that is unsubstituted or substituted with one or more R²⁰. In some embodiments, R^{e} is C₁₋₆alkyl that is unsubstituted. In some embodiments, R^{e} is C₁₋₆alkyl that is substituted with one or more R²⁰. In some embodiments, R^{e} is C₁₋₆alkyl that is substituted with one or more R²⁰ wherein each R²⁰ is independently selected from —OH, —OC₁₋₆alkyl, =O, and —CN.

In some embodiments, for a compound of any one of Formulas I, IA, IB, IC, ID, and IE, R² is H. In some embodiments, R² is a C₁₋₆alkyl that is unsubstituted or is substituted with one or more R¹³. In some embodiments, R² is a C₁₋₆alkyl that is unsubstituted. In some embodiments, R² is a C₁₋₆alkyl that is substituted with one or more R¹³. In some embodiments, R² is a C₁₋₂alkyl that is unsubstituted or is substituted with one or more R¹³. In some embodiments, R² is a C₁₋₂alkyl that is unsubstituted. In some embodiments, R² is a C₁₋₂alkyl that is substituted with one or more R¹³.

In some embodiments, for a compound according to Formula I or IA, R² and R³, together with the nitrogen atom to which they are attached, form a 4-9 membered heterocycle that is unsubstituted or is substituted with one or more R¹¹. In some embodiments, R² and R³, together with the nitrogen atom to which they are attached, form a 4-6 membered heterocycle that is unsubstituted or is substituted with one or more R¹¹. In some embodiments, R² and R³, together with the nitrogen atom to which they are attached, form a 4-6 membered heterocycle that is unsubstituted. In some embodiments, R² and R³, together with the nitrogen atom to which they are attached, form a 4-6 membered heterocycle that is substituted with one or more R¹¹. In some embodiments, R² and R³, together with the nitrogen atom to which they are attached, form a 4-6 membered heterocycle that includes 1 or 2 heteroatoms selected from O, S, and N, wherein the heterocycle is unsubstituted or is substituted with one or more R¹¹. In some embodiments, R² and R³, together with the nitrogen atom to which they are attached, form a 4-6 membered heterocycle that includes 1 or 2 heteroatoms selected from O, S, and N, wherein the heterocycle is unsubstituted. In some embodiments, R² and R³, together with the nitrogen atom to which they are attached, form a 4-6 membered heterocycle that includes 1 or 2 heteroatoms selected from O, S, and N, wherein the heterocycle is substituted with 1-4 R¹¹. In some embodiments, R² and R³, together with the nitrogen atom to which they are attached, form a 4-6 membered heterocycle that includes 2 nitrogen atoms, wherein the heterocycle is unsubstituted or is substituted with one or more R¹¹. In some embodiments, R² and R³, together with the nitrogen atom to which they are attached, form a 4-6 membered heterocycle that includes 2 nitrogen atoms, wherein the heterocycle is unsubstituted. In some embodiments, R² and R³, together with the nitrogen atom to which they are attached, form a 4-6 membered heterocycle that includes 2 nitrogen atoms, wherein the heterocycle is substituted with one or more R¹¹. In some embodiments, R² and R³, together with the nitrogen atom to which they are attached, form a 4-6 membered heterocycle that includes 2 nitrogen atoms, wherein the heterocycle is unsubstituted or is substituted with one or more R¹¹, and wherein the heterocycle includes an —NH— moiety. In some embodiments, R² and R³, together with the nitrogen atom to which they are attached, form a pyrrolidine that is substituted with 0-4 R¹¹. In some embodiments, R² and R³, together with the nitrogen atom to which they are attached, form an azetidine that is substituted with 0-4 R¹¹. In some embodiments, R² and R³, together with the nitrogen atom to which they are attached, form a piperidine that is substituted with 0-4 R¹¹. In some embodiments, R² and R³, together with the nitrogen atom to which they are attached, form a piperazine that is substituted with 0-4 R¹¹. In some embodiments, R² and R³, together with the nitrogen atom to which they are attached, form a piperazine that is unsubstituted. In some embodiments, R² and R³, together with the nitrogen atom to which they are attached, form a piperazine that is substituted with 1-4 R¹¹. In some embodiments, R² and R³, together with the nitrogen atom to which they are attached, form a piperazine that is substituted with 0-4 R¹¹, and wherein the heterocycle includes an —NH— moiety. In some embodiments, R² and R³, together with the nitrogen atom to which they are attached, form a 4-6 membered heterocycle substituted with one or more R¹¹, wherein at least one R¹¹ is selected from —OR¹² and a C₁₋₆alkyl substituted with —OH. In some embodiments, R² and R³, together with the nitrogen atom to which they are attached, form a 4-6 membered heterocycle substituted with one or more R¹¹, wherein at least one R¹¹ is an unsubstituted C₁₋₆alkyl.

In some embodiments, for a compound according to Formula I or IA, R² and R³, together with the nitrogen atom to which they are attached, form a bridged heterocycle that is unsubstituted or is substituted with one or more R¹¹. In some embodiments, R² and R³, together with the nitrogen atom to which they are attached, form a 7-9 membered bridged heterocycle that includes 1 or 2 heteroatoms selected from 0, S, and N, wherein the bridged heterocycle is unsubstituted or is substituted with one or more R¹¹. In some embodiments, R² and R³, together with the nitrogen atom to which they are attached, form a 7-9 membered bridged heterocycle that includes 1 or 2 heteroatoms selected from O, S, and N, wherein the bridged heterocycle is unsubstituted. In some embodiments, R² and R³, together with the nitrogen atom to which they are attached, form a 7-9 membered bridged heterocycle that includes 1 or 2 heteroatoms selected from O, S, and N, wherein the bridged heterocycle is substituted with 1-4 R¹¹. In some embodiments, R² and R³, together with the nitrogen atom to which they are attached, form a 7-9 membered bridged heterocycle that includes 2 nitrogen atoms, wherein the bridged heterocycle is substituted with 0-4 R¹¹. In some embodiments, R² and R³, together with the nitrogen atom to which they are attached, form a bridged piperidine that is substituted with 0-4 R¹¹. In some embodiments, R² and R³, together with the nitrogen atom to which they are attached, form a bridged piperazine that is substituted with 0-4 R¹¹. In some embodiments, R² and R³, together with the nitrogen atom to which they are attached, form a bridged piperazine that is unsubstituted. In some embodiments, R² and R³, together with the nitrogen atom to which they are attached, form a bridged piperazine that is substituted with 1-4 R¹¹. In some embodiments, R² and R³, together with the nitrogen atom to which they are attached, form a bridged piperazine that is substituted with 0-4 R¹¹, wherein the piperazine includes an —NH— moiety. In some embodiments, R² and R³, together with the nitrogen atom to which they are attached, form a bridged heterocycle substituted with one or more R¹¹, wherein at least one R¹¹ is selected from —OR¹² and a C₁₋₆alkyl substituted with —OH. In some embodiments, R² and R³, together with the nitrogen atom to which they are attached, form a bridged heterocycle substituted with one or more R¹¹, wherein at least one R¹¹ is an unsubstituted C₁₋₆alkyl.

In some embodiments, for a compound according to Formula I or IA, R² and R³, together with the nitrogen atom to which they are attached, form a spirocycle that is unsubstituted or is substituted with one or more R¹¹. In some embodiments, R² and R³, together with the nitrogen atom to which they are attached, form a spirocycle that is unsubstituted. In some embodiments, R² and R³, together with the atom to which they are attached, form a spirocycle that is substituted with one or more R¹¹. In some embodiments, R² and R³, together with the nitrogen atom to which they are attached, form a spirocycle that includes an —NH— moiety.

In some embodiments, for a compound according to Formula I or IA, R² and R³, together with the nitrogen atom to which they are attached, form a 4-9 membered heterocycle that is unsubstituted or is substituted with one or more R¹¹, wherein the one or more R¹ are independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹¹)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, R² and R³, together with the nitrogen atom to which they are attached, form a 4-9 membered heterocycle that is unsubstituted or is substituted with one or more R¹¹, wherein the heterocycle includes an —NH— moiety, and wherein the one or more R¹¹ are independently selected from —OR¹², ═O, —C(O)(C₁₋₆alkylene)CN, —C(O)(C₁₋₆alkylene)OR¹¹, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.

In some embodiments, the compound is a compound according to Formula IF:

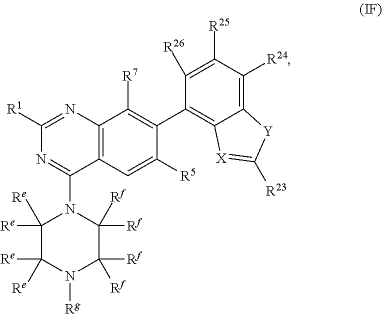

or a salt (e.g., pharmaceutically acceptable salt), ester, tautomer, prodrug, zwitterionic form, or stereoisomer thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R³;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹¹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R²⁰ is independently selected from —OH, —OC₁₋₆alkyl, —CN, —NH₂, —NHC₁₋₆alkyl, and halogen;
each R^{e} and R^{f} is independently selected from R¹¹ and hydrogen, wherein an R^{e} and an R^{f} can optionally join together to form a 4-6 membered ring, or a first R^{f} and a second R^{f} connected to adjacent atoms can optionally join together to form a 3-5 membered ring, or a first R^{e} and a second R^{e} connected to adjacent atoms can optionally join together to form a 3-5 membered ring;
R^{g} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

In some embodiments, the compound is a compound according to Formula IF, or a salt (e.g., pharmaceutically acceptable salt) thereof.

In some embodiments, for a compound according to Formula IF, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R¹²)₂. In some embodiments, X is C—CN, Y is S. and R²³ is —NH₂. In some embodiments, X is C—CN, Y is S, R²³ is —N(R¹²)₂, and R²⁴ is a halogen (e.g., F). In some embodiments, X is C—CN, Y is S, R²³ is —N(R¹²)₂, and one or more of R²⁴, R²⁵, and R²⁶ is a halogen (e.g., F).

In some embodiments, for a compound according to Formula IF,

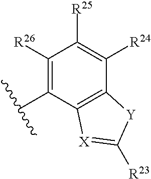

is

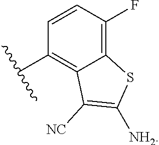 [SMILES: *c1ccc(F)c2sc(N)c(C#N)c12]

In some embodiments, for a compound according to Formula IF, R^{g} is selected from H and C₁₋₆alkyl that is unsubstituted. In some embodiments, R^{g} is H.

In some embodiments, for a compound according to Formula IF, each R^{e} and R^{f} is independently selected from R¹¹ and hydrogen. In some embodiments, each R^{e} and R^{f} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰. In some embodiments, each R^{e} and R^{f} is independently selected from hydrogen, —OR¹², ═O, —C(O)(C₁₋₆alkylene)CN, —C(O)(C₁₋₆alkylene)OR⁴, —C(O)(C₁₋₆alkyl), —S(O)₂(C₁₋₆alkyl), —N(R⁴)C(O)(C₁₋₆alkyl), —N(R⁴)C(O)(C₁₋₆alkylene)OR¹⁴, halogen, and C₁₋₆alkyl that is unsubstituted or substituted with one or more R²⁰. In some embodiments, at least one of R^{e} and R^{f} is —OR² or C₁₋₆alkyl that is unsubstituted or substituted with one or more R²⁰ (e.g., C₁₋₆ alkyl that is unsubstituted or substituted with —OH or —CN). In some embodiments, each R^{e} and R^{f} is hydrogen. In some embodiments, 0-2 R^{e} and R^{f} groups are C₁₋₆ alkyl and the remaining R^{e} and R^{f} groups are H. It will be appreciated that when R^{e} or R^{f} is =O, the other R^{e} or R^{f} on the same carbon atom is absent, such that the carbon has proper valency.

In some embodiments, for a compound according to Formula IF, each R^{e}, R^{f}, and R^{g} is H.

In some embodiments, for a compound according to Formula IF, an R^{e} and an R^{f} join together to form a 4-6 membered ring.

In some embodiments, for a compound according to Formula IF, a first R^{f} and a second R^{f} connected to adjacent atoms join together to form a 3-5 membered ring, or a first R^{e} and a second R^{e} connected to adjacent atoms join together to form a 3-5 membered ring.

In some embodiments, the compound is a compound according to Formula IG, IH, IJ, IK, IL, IM, or IN:

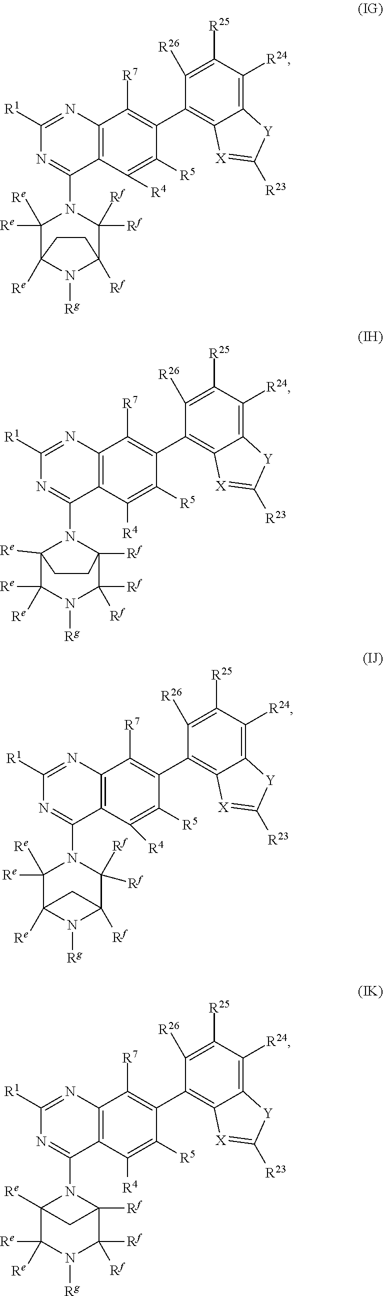

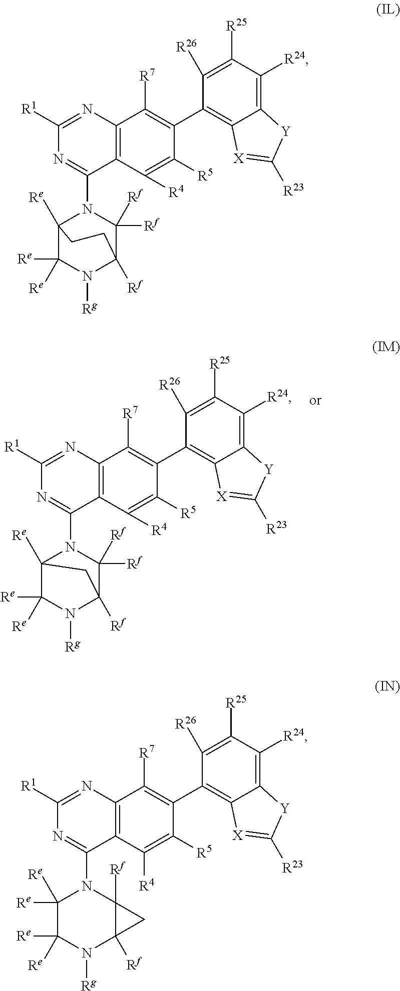

or a salt (e.g., pharmaceutically acceptable salt), ester, tautomer, prodrug, zwitterionic form, or stereoisomer thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹;
each R¹¹ is independently selected from —OR¹², ═O, —C(O)(C₁₋₆alkylene)CN, —C(O)(C₁₋₆ alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆ alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R²⁰ is independently selected from —OH, —OC₁₋₆alkyl, —CN, —NH₂, —NHC₁₋₆alkyl, and halogen;
each R^{e} and R^{f} is independently selected from R¹¹ and hydrogen;
R^{g} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

In some embodiments, the present disclosure provides a compound of Formula IG or a salt (e.g., pharmaceutically acceptable salt), ester, tautomer, prodrug, zwitterionic form, or stereoisomer thereof. In some embodiments, the present disclosure provides a compound of Formula IH or a salt (e.g., pharmaceutically acceptable salt), ester, tautomer, prodrug, zwitterionic form, or stereoisomer thereof. In some embodiments, the present disclosure provides a compound of Formula IJ or a salt (e.g., pharmaceutically acceptable salt), ester, tautomer, prodrug, zwitterionic form, or stereoisomer thereof. In some embodiments, the present disclosure provides a compound of Formula IK or a salt (e.g., pharmaceutically acceptable salt), ester, tautomer, prodrug, zwitterionic form, or stereoisomer thereof. In some embodiments, the present disclosure provides a compound of Formula IL or a salt (e.g., pharmaceutically acceptable salt), ester, tautomer, prodrug, zwitterionic form, or stereoisomer thereof. In some embodiments, the present disclosure provides a compound of Formula IM or a salt (e.g., pharmaceutically acceptable salt), ester, tautomer, prodrug, zwitterionic form, or stereoisomer thereof. In some embodiments, the present disclosure provides a compound of Formula IN or a salt (e.g., pharmaceutically acceptable salt), ester, tautomer, prodrug, zwitterionic form, or stereoisomer thereof.

In some embodiments, the present disclosure provides a compound of Formula IG or a salt (e.g., a pharmaceutically acceptable salt) thereof. In some embodiments, the present disclosure provides a compound of Formula IH or a salt (e.g., a pharmaceutically acceptable salt) thereof. In some embodiments, the present disclosure provides a compound of Formula IJ or a salt (e.g., a pharmaceutically acceptable salt) thereof. In some embodiments, the present disclosure provides a compound of Formula IK or a salt (e.g., a pharmaceutically acceptable salt) thereof. In some embodiments, the present disclosure provides a compound of Formula IL or a salt (e.g., a pharmaceutically acceptable salt) thereof. In some embodiments, the present disclosure provides a compound of Formula IM or a salt (e.g., a pharmaceutically acceptable salt) thereof. In some embodiments, the present disclosure provides a compound of Formula IN or a salt (e.g., a pharmaceutically acceptable salt) thereof.

In some embodiments, for a compound according to any one of Formulas IG, IH, IJ, IK, IL, IM, and IN, X is C—CN and Y is S. In some embodiments, X is C—CN and Y is O. In some embodiments, X is N and Y is S. In some embodiments, X is N and Y is O. In some embodiments, X is C—CN, Y is S, and R²³ is —N(R²)₂. In some embodiments, X is C—CN, Y is S, and R²³ is —NH₂. In some embodiments, X is C—CN, Y is S, R²³ is —N(R¹²)₂, and R²⁴ is a halogen (e.g., F). In some embodiments, X is C—CN, Y is S, R²³ is —N(R¹²)₂, and one or more of R²⁴, R²⁵, and R²⁶ is a halogen (e.g., F).

In some embodiments, for a compound according to any one of Formulas IG, IH, IJ, IK, IL, IM, and IN,

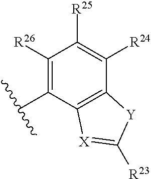

is

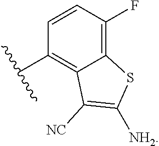 [SMILES: *c1ccc(F)c2sc(N)c(C#N)c12]

In some embodiments, for a compound according to any one of Formulas IG, IH, IJ, IK, IL, IM, and IN, R^{g} is selected from H and C₁₋₆alkyl that is unsubstituted. In some embodiments, R^{g} is H.

In some embodiments for a compound according to any one of Formulas IG, IH, IJ, IK, IL, IM, and IN, each R^{e} and R^{f} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰. In some embodiments, at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN). In some embodiments, each R^{e} and R^{f} is hydrogen. In some embodiments, 0-2 R^{e} and R^{f} groups are C₁₋₆ alkyl and the remaining R^{e} and R^{f} groups are H. It will be appreciated that when R^{e} or R^{f} is =O, the other R^{e} or R^{f} on the same carbon atom is absent, such that the carbon has proper valency.

In some embodiments, for a compound according to any one of Formulas IG, IH, IJ, IK, IL, IM, and IN, each R^{e}, R^{f}, and R^{g} is H.

In some embodiments, for a compound according to any one of Formulas I, IA, IB, IC, ID, IE, IF, IG, IH, IJ, IK, IL, IM, and IN, R⁴ is H. In some embodiments, R⁴ is —OR¹⁴. In some embodiments, R⁴ is —OCH₃.

In some embodiments, for a compound according to any one of Formulas I, IA, IB, IC, ID, IE, IF, IG, IH, IJ, IK, IL, IM, and IN, R⁵ is a halogen (e.g., F or Cl). In some embodiments, R⁵ is Cl. In some embodiments, R⁵ is F. In some embodiments, R⁵ is selected from C₁₋₆alkyl that is unsubstituted or substituted with one or more R¹³. In some embodiments, R⁵ is selected from C₁₋₂alkyl that is unsubstituted or substituted with one or more R¹³. In some embodiments, R⁵ is selected from C₁₋₆alkyl that is unsubstituted, such as methyl or ethyl. In some embodiments, R⁵ is selected from C₁₋₆alkyl that is substituted with one or more halogens or —CN. In some embodiments, R⁵ is C₁₋₆alkyl that is substituted with one or more halogens, such as one or more fluorines. In some embodiments, R⁵ is —CF₃. In some embodiments, R⁵ is —CHF₂. In some embodiments, R⁵ is selected from —CF₂H, —CF₃, —CH₂CN, and —CH₂CH₃. In some embodiments, R is selected from CH₃, —CH₂CH₃, —CF₂H, —CF₃, —CF₂CH₃, and —CH₂CN. In some embodiments, R⁵ is C₁₋₆alkyl that is substituted with one or more R¹³, wherein each R¹³ is independently selected from —OR¹⁴, —CN, and —N(R¹⁴)₂. In some embodiments, R⁵ is —CH₂CN. In some embodiments, R⁵ is —CN. In some embodiments, R⁵ is —OR¹². In some embodiments, R⁵ is —OR¹², where R¹² is selected from C₁₋₆ alkyl and C₂₋₆ alkenyl, wherein any C₁₋₆alkyl or C₂₋₆ alkenyl is unsubstituted or substituted with one or more R¹³. In some embodiments, R⁵ is —OR¹², where R¹² is a C₁₋₆ alkyl that is unsubstituted or substituted with one or more R¹³. In some embodiments, R⁵ is —OR¹², where R¹² is a C₁₋₆ alkyl that is substituted with one or more halogens. In some embodiments, R⁵ is —OCF₃. In some embodiments, R⁵ is —OCH₃. In some embodiments, R⁵ is a 3-6 membered heterocycle. In some embodiments, R⁵ is a 5-6 membered heteroaryl, such as a furan.

In some embodiments, for a compound according to any one of Formulas I, IA, IB, IC, ID, IE, IF, IG, IH, IJ, IK, IL, IM, and IN, R⁷ is Cl. In some embodiments, R⁷ is F.

In some embodiments, for a compound of according to any one of Formulas I, IA, IB, IC, ID, IE, IF, IG, IH, IJ, IK, IL, IM, and IN, R¹ is selected from C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷. In some embodiments, R¹ is selected from C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷, provided that R⁸ is not an unsubstituted or substituted heterocycle selected from:

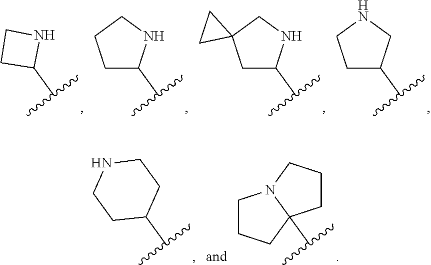 [SMILES: *C1CCN1 ; *C1CCCN1 ; *C1CC2(CC2)CN1 ; *C1CCNC1 ; *C1CCNCC1 ; *C12CCCN1CCC2]

In some embodiments, R¹ is selected from C₁₋₆alkyl, —OR⁸, —O(C₁₋₆alkylene)R⁸, —N(R¹⁶)₂, and a 4-, 5- or 7-membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷.

In some embodiments, for a compound of according to any one of Formulas I, IA, IB, IC, ID, IE, IF, IG, IH. IJ, IK, IL, IM, and IN, R¹ is H.

In some embodiments, for a compound according to any one of Formulas I, IA, IB, IC, ID, IE, IF, IG, IH, IJ, IK, IL, IM, and IN, R¹ is C₁₋₆alkyl that is unsubstituted or substituted with one or more R¹³. In some embodiments, R¹ is C₁₋₃alkyl that is unsubstituted or substituted with one or more R¹³. In some embodiments, R¹ is C₁₋₃alkyl that is unsubstituted.

In some embodiments, for a compound according to any one of Formulas I, IA, IB, IC, ID, IE, IF, IG, IH, IJ, TK, IL, IM, and TN, R¹ is —OR⁸ or —O(C₁₋₆alkylene)R⁸, wherein any C₁₋₆alkylene is unsubstituted or substituted with one or more R¹³. In some embodiments, R¹ is —OR⁸. In some embodiments, R¹ is —O(C₁₋₆ alkylene)R⁸, wherein any C₁₋₆alkylene is unsubstituted or substituted with one or more R¹³. In some embodiments, R¹ is —O(C₁₋₆alkylene)Rx, wherein any C₁₋₆alkylene is unsubstituted. In some embodiments, R¹ is —OH. In some embodiments, R¹ is —O(C₁₋₆alkylene)H, wherein any C₁₋₆alkylene is unsubstituted or substituted with one or more R¹³. In some embodiments, R¹ is —OR⁸ and R⁸ is C₁₋₆alkyl that is unsubstituted or substituted with one or more R¹³. In some embodiments, R¹ is —OR⁸ and R⁸ is C₁₋₆alkyl that is unsubstituted. In some embodiments, R¹ is —OR⁸ and R⁸ is C₁₋₃alkyl that is unsubstituted or substituted with one or more R¹³. In some embodiments, R¹ is —OR⁸ and R⁸ is C₁₋₃alkyl that is unsubstituted.

In some embodiments, for a compound according to any one of Formulas I, IA, IB, IC, ID, IE, IF, IG, IH, IJ. IK, IL, IM, and IN, R¹ is selected from

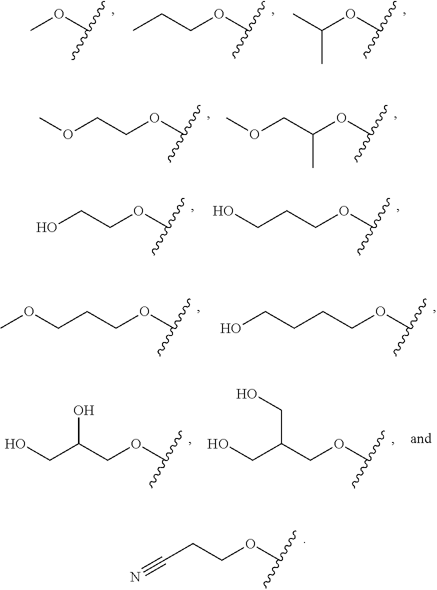

In some embodiments, for a compound according to any one of Formulas I, IA, IB, IC, ID, IE, IF, IG, IH, IJ, IK, IL, IM, and IN, R¹ is —OR⁸ or —O(C₁₋₆alkylene)R⁸, wherein any C₁₋₆alkylene is unsubstituted or substituted with one or more R¹³, and wherein R⁸ is selected from a carbocycle, aryl, heteroaryl, and heterocycle, wherein any carbocycle, aryl, heteroaryl, or heterocycle comprises 3-8 members and is unsubstituted or substituted with one or more R¹⁹. In some embodiments, R¹ is —OR⁸ wherein R⁸ is selected from a carbocycle, aryl, heteroaryl, and heterocycle, wherein any carbocycle, aryl, heteroaryl, or heterocycle comprises 3-8 members and is unsubstituted or substituted with one or more R¹⁹. In some embodiments, R¹ is —O(C₁₋₆alkylene)R⁸, wherein any C₁₋₆alkylene is unsubstituted or substituted with one or more R¹³, and wherein R⁸ is selected from a carbocycle, aryl, heteroaryl, and heterocycle, wherein any carbocycle, aryl, heteroaryl, or heterocycle comprises 3-8 members and is unsubstituted or substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is selected from a carbocycle, aryl, heteroaryl, and heterocycle, wherein any carbocycle, aryl, heteroaryl, or heterocycle comprises 3-8 members and is unsubstituted or substituted with one or more R¹⁹, provided that R⁸ is not an unsubstituted or substituted heterocycle selected from:

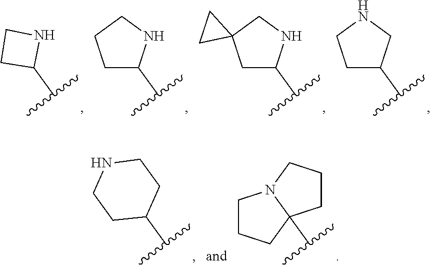 [SMILES: *C1CCN1 ; *C1CCCN1 ; *C1CC2(CC2)CN1 ; *C1CCNC1 ; *C1CCNCC1 ; *C12CCCN1CCC2]

In some embodiments, for a compound according to any one of Formulas I, IA, IB, IC, ID, IE, IF, IG, IH, IJ, IK, IL, IM, and IN, R¹ is —OR⁸ or —O(C₁₋₆alkylene)R⁸, wherein any C₁₋₆alkylene is unsubstituted or substituted with one or more R¹³, and wherein R⁸ is a heteroaryl that comprises 3-8 members (e.g., 5-8 members) and is unsubstituted or substituted with one or more R¹⁹. In some embodiments, R⁸ is a heteroaryl that comprises 3-8 members (e.g., 5-8 members) including one or more heteroatoms selected from N, 0, and S, wherein the heteroaryl is unsubstituted or substituted with one or more R¹⁹. In some embodiments, R⁸ is a heteroaryl that comprises 3-8 members (e.g., 5-8 members) including one or more heteroatoms selected from N, O, and S, wherein the heteroaryl is unsubstituted. In some embodiments, R⁸ is a heteroaryl that comprises 3-8 members (e.g., 5-8 members) including one or more heteroatoms selected from N, O, and S, wherein the heteroaryl is substituted with one or more R¹⁹. In some embodiments, R⁸ is a heteroaryl that comprises 3-8 members (e.g., 5-8 members) including two or more heteroatoms selected from N, O, and S, wherein the heteroaryl is unsubstituted or substituted with one or more R¹⁹. In some embodiments, R⁸ is a heteroaryl that comprises 3-8 members (e.g., 5-8 members) including two or more heteroatoms selected from N, O, and S, wherein the heteroaryl is unsubstituted. In some embodiments, R⁸ is a heteroaryl that comprises 3-8 members (e.g., 5-8 members) including two or more heteroatoms selected from N, O, and S, wherein the heteroaryl is substituted with one or more R¹⁹. In some embodiments, R⁸ is a heteroaryl that comprises 3-8 members (e.g., 5-8 members) including two nitrogen atoms, wherein the heteroaryl is unsubstituted or substituted with one or more R¹⁹. In some embodiments, R⁸ is a heteroaryl that comprises 3-8 members (e.g., 5-8 members) including two nitrogen atoms, wherein the heteroaryl is unsubstituted. In some embodiments, R⁸ is a heteroaryl that comprises 3-8 members (e.g., 5-8 members) including two nitrogen atoms, wherein the heteroaryl is substituted with one or more R¹⁹.

In some embodiments, for a compound according to any one of Formulas I, IA, IB, IC, ID, IE, IF, IG, IH, IJ, IK, IL, IM, and IN, R¹ is

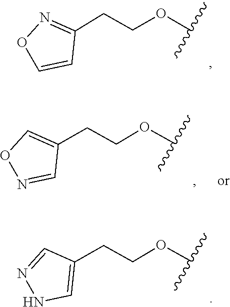

In some embodiments, for a compound according to any one of Formulas I, IA, IB, IC, ID, IE, IF, IG, IH, IJ, IK, IL, IM, and IN, R¹ is —OR⁸ or —O(C₁₋₆alkylene)R⁸, wherein any C₁₋₆alkylene is unsubstituted or substituted with one or more R¹³, and wherein R⁸ is a heterocycle that comprises 3-8 members and is unsubstituted or substituted with one or more R¹⁹. In some embodiments, R⁸ is a heterocycle that comprises 3-8 members including one or more heteroatoms selected from N, O, and S, wherein the heterocycle is unsubstituted or substituted with one or more R¹⁹. In some embodiments, R⁸ is a heterocycle that comprises 3-8 members including one or more heteroatoms selected from N, O, and S, wherein the heterocycle is unsubstituted. In some embodiments, R⁸ is a heterocycle that comprises 3-8 members including one or more heteroatoms selected from N, O, and S, wherein the heterocycle is substituted with one or more R¹⁹. In some embodiments, R⁸ is a heterocycle that comprises 3-8 members including two or more heteroatoms selected from N, O, and S, wherein the heterocycle is unsubstituted or substituted with one or more R¹⁹. In some embodiments, R⁸ is a heterocycle that comprises 3-8 members including two or more heteroatoms selected from N, O, and S, wherein the heterocycle is unsubstituted. In some embodiments, R⁸ is a heterocycle that comprises 3-8 members including two or more heteroatoms selected from N, O, and S, wherein the heterocycle is substituted with one or more R¹⁹. In some embodiments, R⁸ is a heterocycle that comprises 3-8 members including three or more heteroatoms selected from N, O, and S, wherein the heterocycle is unsubstituted or substituted with one or more R¹⁹. In some embodiments, R⁸ is a heterocycle that comprises 3-8 members including three or more heteroatoms selected from N, O, and S, wherein the heterocycle is unsubstituted. In some embodiments, R⁸ is a heterocycle that comprises 3-8 members including three or more heteroatoms selected from N, O, and S, wherein the heterocycle is substituted with one or more R¹⁹. In some embodiments, R⁸ is a heterocycle that comprises 3-8 members including two oxygen atoms, wherein the heterocycle is unsubstituted or substituted with one or more R¹⁹. In some embodiments, R⁸ is a heterocycle that comprises 3-8 members including two oxygen atoms, wherein the heterocycle is unsubstituted. In some embodiments, R⁸ is a heterocycle that comprises 3-8 members including two oxygen atoms, wherein the heterocycle is substituted with one or more R¹⁹.

In some embodiments, for a compound according to any one of Formulas I, IA, IB, IC, ID, IE, IF, IG, IH, IJ, IK, IL, IM, and TN, R¹ is selected from

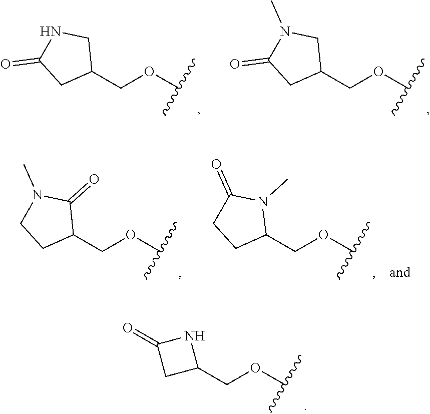

In some embodiments, for a compound according to any one of Formulas I, IA, IB, IC, ID, IE, IF, IG, IH, IJ, IK, IL, IM, and IN, R¹ is selected from

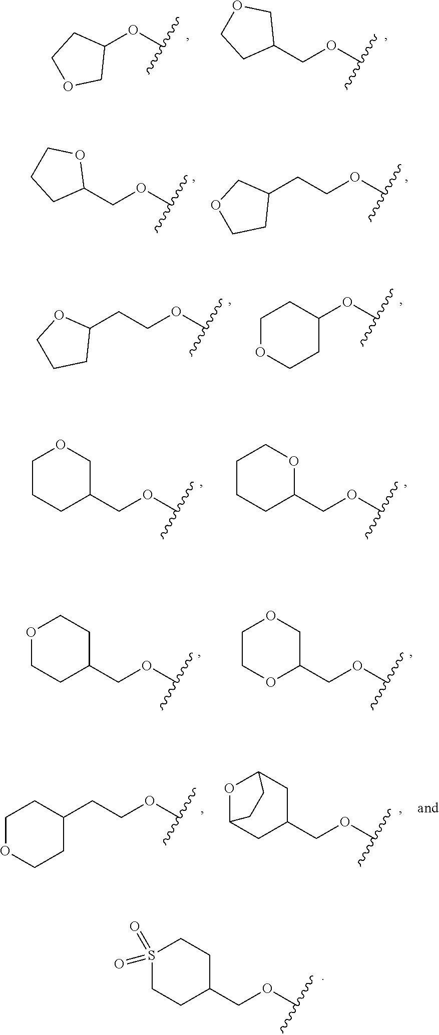

In some embodiments, for a compound according to any one of Formulas I, IA, IB, IC, ID, IE, IF, IG, IH, IJ, IK, IL, IM, and IN, R¹ is not a structure selected from:

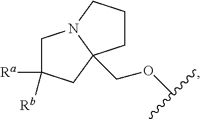

wherein R^{a} and R^{b} are each independently selected from halogen, C₁₋₆alkyl, —OR¹², and H, wherein any C₁₋₆alkyl is unsubstituted or is substituted with one or more R¹³. In some embodiments, R¹ is not a structure selected from:

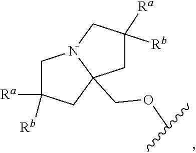

wherein R^{a} and R^{b} are each independently selected from deuterium, halogen, C₁₋₆alkyl, —OR¹², and H, wherein any C₁₋₆alkyl is unsubstituted or is substituted with one or more R¹³, or an R^{a} and R^{b} on the same atom combine to form a 3-membered carbocycle optionally substituted with one or more halogen. In some embodiments, R¹ is not a structure selected from:

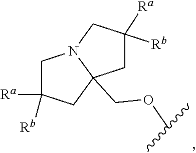

wherein R^{a} and R^{b} are each independently selected from deuterium, halogen, C₁₋₆alkyl, —OR¹², and H, wherein any C₁₋₆alkyl is unsubstituted or is substituted with one or more R¹³.

In some embodiments, for a compound according to any one of Formulas I, IA, IB, IC, ID, IE, IF, IG, IH, IJ, IK, IL, IM, and IN, R¹ is not a structure selected from:

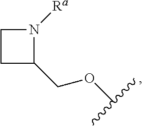

wherein R^{a} is selected from halogen, C₁₋₆alkyl, —OR¹², and H, wherein any C₁₋₆alkyl is unsubstituted or is substituted with one or more R¹.

In some embodiments, for a compound according to any one of Formulas I, IA, IB, IC, ID, IE, IF, IG, IH, IJ, IK, IL, IM, and IN, R¹ is not a structure selected from:

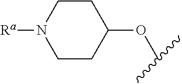

wherein R^{a} is selected from C₁₋₆alkyl and H, wherein any C₁₋₆alkyl is unsubstituted or is substituted with one or more R¹³.

In some embodiments, for a compound according to any one of Formulas I, IA, IB, IC, ID, IE, IF, IG, IH, IJ, IK, IL, IM, and IN, R¹ is not a structure selected from:

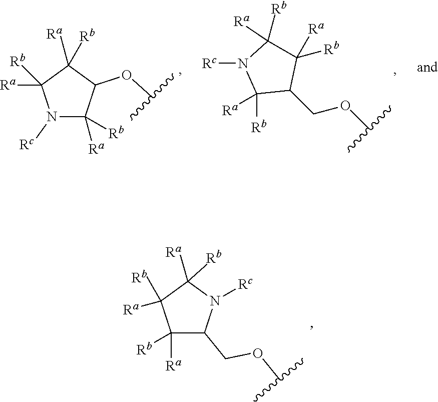

wherein each R^{a} and R^{b} is independently selected from halogen, C₁₋₆ alkyl, —OR¹², and H; and R^{c} is selected from C₁₋₆ alkyl, wherein the C₁₋₆ alkyl is unsubstituted or is substituted with one or more R¹³. In some embodiments, R¹ is not a structure selected from:

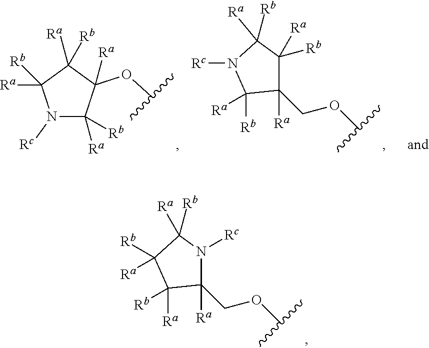

wherein each R^{a} and R^{b} is independently selected from halogen, C₁₋₆ alkyl, —OR², and H; and R is selected from C₁₋₆ alkyl, wherein the C₁₋₆ alkyl is unsubstituted or is substituted with one or more R¹³, or an R^{a} and R^{b} on the same atom combine to form a 3-membered carbocycle.

In some embodiments, for a compound according to any one of Formulas I, IA, IB, IC, ID, IE, IF, IG, IH, IJ, IK, IL, IM, and IN, R¹ is not a structure selected from:

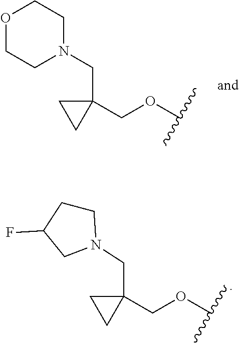 [SMILES: *OCC1(CN2CCOCC2)CC1 ; *OCC1(CN2CCC(F)C2)CC1]

In some embodiments, for a compound according to any one of Formulas I, IA, IB, IC, ID, IE, IF, IG, IH, IJ, IK IL, IM, and IN, R¹ is not:

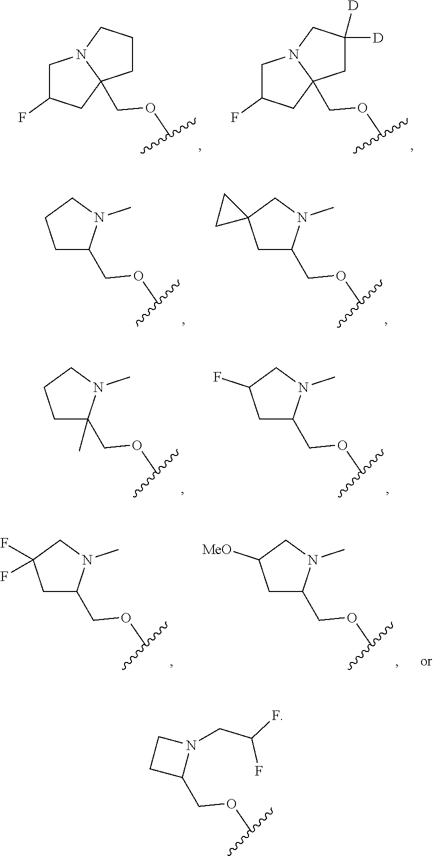

In some embodiments, for a compound according to any one of Formulas I, IA, IB, IC, ID, IE, IF, IG, IH, IJ, IK, IL, IM, and IN, R¹ is —OR⁸ or —O(C₁₋₆alkylene)R⁸, wherein any C₁₋₆alkylene is unsubstituted or substituted with one or more R¹³, and wherein R⁸ is an aryl that comprises 3-8 members (e.g., phenyl) and is unsubstituted or substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is an aryl that comprises 3-8 members (e.g., phenyl) and is unsubstituted. In some embodiments, R¹ is —OR⁸ or —O(C₁₋₆alkylene)R⁸, wherein any C₁₋₆alkylene is unsubstituted or substituted with one or more R¹³, and wherein R⁸ is an aryl that comprises 3-8 members (e.g., phenyl) and is substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is a phenyl that is unsubstituted or substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is a phenyl that is unsubstituted. In some embodiments, R¹ is

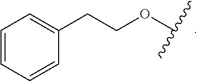 [SMILES: *OCCc1ccccc1]

In some embodiments, for a compound according to any one of Formulas I, IA, IB, IC, ID, IE, IF, IG, IH, IJ, IK, IL, IM, and IN, R¹ is —OR⁸ or —O(C₁₋₆alkylene)R⁸, wherein any C₁₋₆alkylene is unsubstituted or substituted with one or more R¹³, and wherein R⁸ is a carbocycle that comprises 3-8 members and is unsubstituted or substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is a carbocycle that comprises 3-8 members and is unsubstituted. In some embodiments, R¹ is —OR⁸ or —O(C₁₋₆alkylene)R⁸, wherein any C₁₋₆alkylene is unsubstituted or substituted with one or more R¹³, and wherein R⁸ is a carbocycle that comprises 3-8 members and is substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is a carbocycle that comprises 3-6 members and is unsubstituted or substituted with one or more R¹⁹. In some embodiments, R¹ is —OR⁸ or —O(C₁₋₆alkylene)R⁸, wherein any C₁₋₆alkylene is unsubstituted or substituted with one or more R¹³, and wherein R⁸ is a carbocycle that comprises 3-6 members and is unsubstituted.

In some embodiments, for a compound according to any one of Formulas I, IA, IB, IC, ID, IE, IF, IG, IH, IJ, IK, IL, IM, and IN, R¹ is selected from

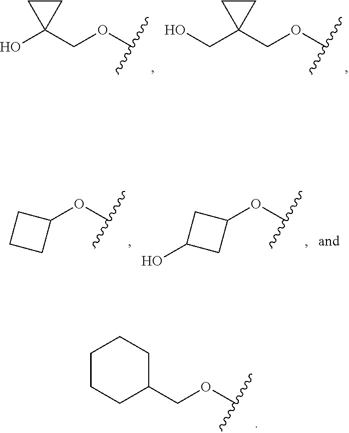 [SMILES: *OCC1(O)CC1 ; *OCC1(CO)CC1 ; *OC1CCC1 ; *OC1CC(O)C1 ; *OCC1CCCCC1]

In some embodiments, for a compound according to any one of Formulas I, IA, IB, IC, ID, IE, IF, IG, IH, IJ, IK, IL, IM, and IN, R¹ is —N(R¹⁶)₂. In some embodiments, R¹ is —NHR¹⁶. In some embodiments, each R¹⁶ is independently selected from H, C₁₋₆alkyl, —C(O)C₁₋₆alkyl, and —C(O)(C₁₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸. In some embodiments, R¹⁶ is selected from C₁₋₆alkyl that is unsubstituted or substituted with one or more R¹³. In some embodiments, each R¹⁶ is independently selected from C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸. In some embodiments, each R¹⁶ is independently selected from C₁₋₆alkyl, —C(O)C₁₋₆alkyl, and —C(O)(C₁₋₆alkylene)heteroaryl, wherein any C₁₋₆alkyl and C₁₋₆ alkylene are unsubstituted or substituted with one or more R¹³, and wherein any heteroaryl is unsubstituted or substituted with one or more R¹⁸. In some embodiments, each R¹⁶ is independently selected from C₁₋₆ alkyl and —C(O)C₁₋₆alkyl, wherein any C₁₋₆alkyl is unsubstituted or substituted with one or more R¹³.

In some embodiments, for a compound according to any one of Formulas I, IA, IB, IC, ID, IE, IF, IG, IH, IJ, IK, IL, IM, and IN, R¹ is selected from

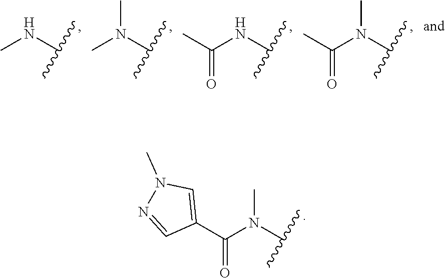

In some embodiments, for a compound according to any one of Formulas I, IA, IB, IC, ID, IE, IF, IG, IH, IJ, IK, IL, IM, and IN, R¹ is a 4-7 membered nitrogen-containing heterocycle that is unsubstituted or substituted with one or more R¹⁷. In some embodiments, R¹ is a 4-7 membered nitrogen-containing heterocycle that is unsubstituted or substituted with —OH. In some embodiments, R¹ is a 4-7 membered nitrogen-containing heterocycle that is unsubstituted. In some embodiments, R¹ is a 4-5 membered nitrogen-containing heterocycle that is unsubstituted. In some embodiments, R¹ is a 4-, 5-, or 7-membered nitrogen-containing heterocycle that is unsubstituted or substituted with one or more R¹⁷.

In some embodiments, for a compound according to any one of Formulas I, IA, IB, IC, ID, IE, IF, IG, IH, IJ, IK, IL, IM, and IN, R¹ is selected from

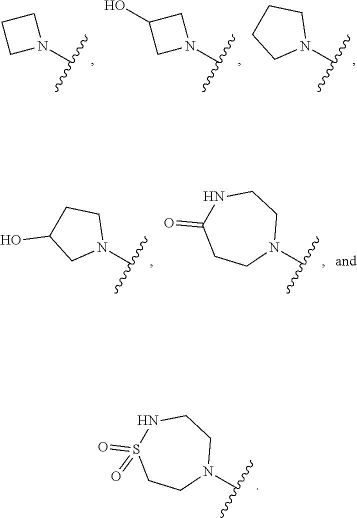

In some embodiments for a compound according to any one of Formulas I, IA, IB, IC, ID, IE, IF, IG, IH, IJ, IK, IL, IM, and IN, R¹ is not

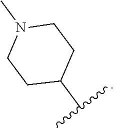 [SMILES: *C1CCN(C)CC1]

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combine to form a ring is mutually exclusive with an embodiment in which one group is ethyl and the other group is hydrogen. Similarly, an embodiment wherein one group is CH₂ is mutually exclusive with an embodiment wherein the same group is NH.

In some embodiments of any of the preceding aspects, the compound is a compound included in Table 2, or a salt, ester, tautomer, prodrug, zwitterionic form, or stereoisomer thereof. In some embodiments, the compound is a compound included in Table 2, or a salt (e.g., a pharmaceutically acceptable salt) thereof. In some embodiments of any of the preceding aspects, the compound is a compound included in Table 3, or a salt, ester, tautomer, prodrug, zwitterionic form, or stereoisomer thereof. In some embodiments, the compound is a compound included in Table 3, or a salt (e.g., a pharmaceutically acceptable salt) thereof.

Also provided herein is a compound selected from Table 2, Table 3, or any of the Examples provided herein, or a salt, ester, tautomer, prodrug, zwitterionic form, or stereoisomer thereof. In some embodiments, the present disclosure provides a compound selected from Table 2, Table 3, or any of the Examples provided herein, or a salt thereof.

In some embodiments of any of the preceding aspects, a compound provided herein, or a salt, ester, tautomer, zwitterionic form, or stereoisomer thereof, is capable of interacting with a residue at the 12 and/or 13 and/or 61 position of the KRAS protein (e.g., a glutamine, histidine, cysteine, valine, aspartic acid, serine, alanine, arginine, or glycine residue). In some embodiments, a compound provided herein, or a salt, ester, tautomer, zwitterionic form, or stereoisomer thereof, is capable of non-covalently interacting with a residue at the 12 and/or 13 and/or 61 position of the KRAS protein (e.g., a glutamine, histidine, cysteine, valine, aspartic acid, serine, alanine, arginine, or glycine residue), such as via one or more van der Waals, hydrogen bonding, ionic, or other interactions.

In some embodiments of any of the preceding aspects, a compound provided herein, or a salt, ester, tautomer, zwitterionic form, or stereoisomer thereof, binds selectively to KRAS having a G12D mutation relative to KRAS having other residues at the 12 position of the P loop, such as cysteine (C), glycine (G), arginine (R), valine (V), serine (S), and alanine (A). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100-fold, or greater selectivity for KRAS having a G12D mutation relative to KRAS having other residues at the 12 position of the P loop, such as cysteine (C), glycine (G), arginine (R), valine (V), serine (S), and alanine (A). In some embodiments of any of the preceding aspects, a compound provided herein, or a salt, ester, tautomer, zwitterionic form, or stereoisomer thereof, binds selectively to KRAS having a G12D mutation relative to wild-type KRAS. For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100-fold, or greater binding selectivity for KRAS having a G12D mutation relative to wild-type KRAS. In some embodiments of any of the preceding aspects, a compound provided herein, or a salt, ester, tautomer, zwitterionic form, or stereoisomer thereof, binds selectively to KRAS having a G12D mutation relative to other forms of RAS (e.g., HRAS and NRAS). For example, in some embodiments, a compound provided herein, or a salt, ester, tautomer, zwitterionic form, or stereoisomer thereof, demonstrates at least 1.5, 2, 3, 4, 5, 10, 20, 30, 40, 50, 60, 70, 80, 90, 100-fold, or greater binding selectivity for KRAS having a G12D mutation relative to another form of RAS (e.g., HRAS or NRAS), such as an HRAS or NRAS protein having a G12D mutation. In some embodiments of any of the preceding aspects, a compound provided herein, or a salt, ester, tautomer, zwitterionic form, or stereoisomer thereof, is capable of binding to a KRAS protein having a G12D mutation and one or more additional mutations, such as a mutation at codon 13 (to, e.g., D or C) or codon 61.

In some embodiments of any of the preceding aspects, a compound provided herein, or a salt, ester, tautomer, zwitterionic form, or stereoisomer thereof, is capable of selectively binding a KRAS protein in an active (GTP-bound) conformation. In some embodiments of any of the preceding aspects, a compound provided herein, or a salt, ester, tautomer, zwitterionic form, or stereoisomer thereof, is capable of selectively binding a KRAS protein in an inactive (GDP-bound) conformation. In some embodiments of any of the preceding aspects, a compound provided herein, or a salt, ester, tautomer, zwitterionic form, or stereoisomer thereof, is capable of selectively binding a KRAS protein in both active (GTP-bound) and inactive (GDP-bound) conformations. In some embodiments of any of the preceding aspects, a compound provided herein, or a salt, ester, tautomer, zwitterionic form, or stereoisomer thereof, has higher selectivity for a KRAS protein in its active (GTP-bound) conformation than in its inactive (GDP-bound) conformation. In some embodiments of any of the preceding aspects, a compound provided herein, or a salt, ester, tautomer, zwitterionic form, or stereoisomer thereof, has higher selectivity for a KRAS protein in its inactive (GDP-bound) conformation than in its active (GTP-bound) conformation.

### Compositions

The present disclosure also provides a composition (e.g., a pharmaceutical composition) comprising a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof. In some embodiments, a provided composition comprises a compound provided herein, or a pharmaceutically acceptable salt thereof. For example, the present disclosure provides a pharmaceutical composition comprising a compound provided herein (e.g., a compound of any one of Formulas I, IA, IB, IC, ID, IE, IF, IG, IH, IJ, TK, IL, IM, and TN), or a salt, ester, tautomer, prodrug, zwitterionic form, or stereoisomer thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, IA, IB, IC, ID, IE, IF, IG, IH, IJ, IK, IL, IM, and IN), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, IA, IB, IC, ID, IE, IF, IG, IH, IJ, IK, IL, IM, and IN), or a pharmaceutically acceptable salt, ester, amide, prodrug or solvate thereof (“active ingredient”) in contact with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, IA, IB, IC, ID, IE, IF, IG, IH, IJ, IK, IL, IM, and IN in any available form (e.g., salt, ester, tautomer, prodrug, zwitterionic form, stereoisomer etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, sealed capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compounds may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, IA, IB, IC, ID, IE, IF, IG, IH, IJ, IK, IL, IM, and IN), or a form thereof (e.g., salt, ester, tautomer, prodrug, zwitterionic form, stereoisomer, etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, IA, IB, IC, ID, IE, IF, IG, IH, IJ, IK, IL, IM, and IN), or a form thereof (e.g., salt, ester, tautomer, prodrug, zwitterionic form, stereoisomer etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, IA, IB, IC, ID, IE, IF, IG, IH, IJ, IK, IL, IM, and IN in any suitable form (e.g., salt, ester, tautomer, prodrug, zwitterionic form, stereoisomer, etc.)), may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, IA, IB, IC, ID, IF, IF, IG, IH, IJ, IK, IL, IM, and IN) or a form thereof (e.g., salt, ester, tautomer, prodrug, zwitterionic form, stereoisomer, etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer, etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, IA, IB, IC, ID, IE, IF, IG, IH, IJ, IK, IL, IM, and IN) or a form thereof (e.g., salt, ester, tautomer, prodrug, zwitterionic form, stereoisomer, etc.) may be formulated for non-systemic administration, such as topical administration. This includes the application of a compound disclosed herein, or a form thereof, externally to the epidermis or the buccal cavity and the instillation of such a compound, or a form thereof, into the ear, eye, and nose, such that the compound, or a form thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments, or pastes, and drops suitable for administration to the eve, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, IA, IB, IC, ID, IE, IF, IG, IH, IJ, IK, IL, IM, and IN) or forms thereof (e.g., salt, ester, tautomer, prodrug, zwitterionic form, stereoisomer, etc.) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as described herein, or an appropriate fraction thereof, of the active ingredient (e.g., a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof).

It should be understood that in addition to the ingredients particularly described elsewhere herein, the formulations described herein may include other useful agents having regard to the type of formulation in question, for example those suitable for oral administration may include flavoring agents.

Compounds (e.g., compounds of any one of Formulas I, IA, IB, IC, ID, IE, IF, IG, IH, IJ, IK, IL, IM, and IN) or forms thereof (e.g., salt, ester, tautomer, prodrug, zwitterionic form, stereoisomer, etc.) may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that maybe combined with the carrier materials to produce a single dosage form will vary depending upon the host treated and the particular mode of administration.

### Methods

The present disclosure also provides a method of modulating KRAS (e.g., KRAS having a Q61H, G12C, G12D, G12V, G12S, G12A, G12R, or G13D mutation, or wild-type KRAS) comprising contacting KRAS with a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof. For example, the present disclosure may provide a method of altering a cell phenotype, cell proliferation, KRAS activity, biochemical output produced by active or inactive KRAS, expression of KRAS, and/or binding of KRAS with a natural binding partner. Any such feature may be monitored and may be altered upon contacting KRAS with a compound provided herein, or a form thereof. A method of modulating KRAS (e.g., KRAS having a Q61H, G12C, G12D, G12V, G12S, G12A, G12R, or G13D mutation, or wild-type KRAS) may be a mode of treatment of a disease, disorder, or condition (e.g., a cancer), a biological assay, a cellular assay, a biochemical assay, etc. In some embodiments, a method of modulating KRAS (e.g., KRAS having a Q61H, G12C, G12D, G12V, G12S, G12A, G12R, or G13D mutation, or wild-type KRAS) comprises contacting a KRAS protein with a compound provided herein, or a salt, ester, tautomer, zwitterionic form, or stereoisomer thereof, where the KRAS protein is in the active (GTP-bound) conformation. In some embodiments, a method of modulating KRAS (e.g., KRAS having a Q61H, G12C, G12D, G12V, G12S, G12A, G12R, or G13D mutation, or wild-type KRAS) comprises contacting a KRAS protein with a compound provided herein, or a salt, ester, tautomer, zwitterionic form, or stereoisomer thereof, where the KRAS protein is in the inactive (GDP-bound) conformation. In some embodiments, contacting a KRAS protein with a compound provided herein, or a salt, ester, tautomer, zwitterionic form, or stereoisomer thereof, comprises incubating the KRAS protein with the compound or form thereof. In some embodiments, contacting a KRAS protein with a compound provided herein, or a salt, ester, tautomer, zwitterionic form, or stereoisomer thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a Q61H, G12C, G12D, G12V, G12S, G12A, G12R, or G13D mutation, or wild-type KRAS.

The present disclosure also provides methods of treating a disease, disorder, or condition in a subject in need thereof using a compound provided herein, (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof. For example, the present disclosure provides a method comprising providing (e.g., administering) to a subject (e.g., patient) in need thereof an effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof. The present disclosure also provides methods of treating a disease, disorder, or condition in a subject in need thereof using a pharmaceutical composition comprising a compound provided herein, (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof. For example, the present disclosure provides a method comprising providing (e.g., administering) to a subject (e.g., patient) in need thereof a pharmaceutical composition comprising an effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof. In some embodiments, the subject is known to have (e.g., has previously been diagnosed with) a disease, disorder, or condition such as a cancer. The disease, disorder, or condition may be a KRAS-mediated disease, such as a cancer characterized by a KRAS protein having a Q61H, G12C, G12D, G12V, G12S, G12A, G12R, or G13D mutation, or wild-type KRAS. In some embodiments, the compound administered to the subject in need thereof according to the methods described herein is a compound described in an embodiment, example, figure, or table herein, or a stereoisomer or pharmaceutically acceptable salt thereof.

The present disclosure also provides a compound as provided herein (e.g., a compound of any one of Formulas T, TA, TB, IC, ID, IE, IF, IG, IH, IJ, 1K, IL, IM, and IN), or a salt, ester, tautomer, prodrug, zwitterionic form, or stereoisomer thereof, or a pharmaceutical composition comprising any of the foregoing compounds and a pharmaceutically acceptable excipient, for use as a medicament, such as a medicament for the treatment of a disease, disorder, or condition (e.g., a cancer). The present disclosure also provides a compound as provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for the treatment of a disease, disorder, or condition (e.g., a cancer) in a subject in need thereof.

The present disclosure also provides the use of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, for the treatment of a disease, disorder, or condition (e.g., a cancer, as described herein, such as a cancer characterized by a KRAS protein having a Q61H, G12C, G12D, G12V, G12S, G12A. G12R, or G13D mutation, or wild-type KRAS) in a subject in need thereof.

The present disclosure also provides the use of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for treating a disease, disorder, or condition (e.g., a cancer, as described herein, such as a cancer characterized by a KRAS protein having a Q61H, G12C, G12D, G12V, G12S, G12A, G12R, or G13D mutation, or wild-type KRAS) in a subject in need thereof.

The present disclosure also provides a method of inhibiting KRAS (e.g., KRAS protein having a Q61H, G12C, G12D, G12V, G12S, G12A, G12R, or G13D mutation, or wild-type KRAS) (e.g., in a subject in need thereof) comprising contacting KRAS with a compound as provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient. In some embodiments, a method of inhibiting KRAS (e.g., KRAS protein having a Q61H, G12C, G12D, G12V, G12S, G12A, G12R, or G13D mutation, or wild-type KRAS) comprises contacting a KRAS protein with a compound provided herein, or a salt, ester, tautomer, zwitterionic form, or stereoisomer thereof, where the KRAS protein is in the active (GTP-bound) conformation. In some embodiments, a method of inhibiting KRAS (e.g., KRAS protein having a Q61H, G12C, G12D, G12V, G12S, G12A, G12R, or G13D mutation, or wild-type KRAS) comprises contacting a KRAS protein with a compound provided herein, or a salt, ester, tautomer, zwitterionic form, or stereoisomer thereof, where the KRAS protein is in the inactive (GDP-bound) conformation. In some embodiments, contacting a KRAS protein with a compound provided herein, or a salt, ester, tautomer, zwitterionic form, or stereoisomer thereof, comprises incubating the KRAS protein with the compound or form thereof. In some embodiments, contacting a KRAS protein with a compound provided herein, or a salt, ester, tautomer, zwitterionic form, or stereoisomer thereof, comprises contacting a cell containing the KRAS protein with the compound or form thereof. In some embodiments, the cell is in a subject. In some embodiments, the subject is a human. In some embodiments, the subject is a human having a disease, disorder, or condition such as a cancer, such as a cancer characterized by a KRAS protein having a Q61H, G12C, G12D, G12V, G12S, G12A, G12R, or G13D mutation, or wild-type KRAS.

The present disclosure also provides a compound as provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, for use in inhibiting KRAS (e.g., KRAS protein having a Q61H, G12C, G12D, G12V, G12S, G12A, G12R, or G13D mutation, or wild-type KRAS) (e.g., in a subject in need thereof). The present disclosure also provides a compound as provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, for use in the manufacture of a medicament for inhibiting KRAS (e.g., KRAS protein having a Q61H, G12C, G12D, G12V, G12S, G12A, G12R, or G13D mutation, or wild-type KRAS) in a subject in need thereof.

The present disclosure also provides the use of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, for inhibiting KRAS (e.g., KRAS protein having a Q61H, G12C, G12D, G12V, G12S, G12A, G12R, or G13D mutation, or wild-type KRAS) in a subject in need thereof.

The present disclosure also provides the use of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, or a pharmaceutical composition comprising any of the foregoing compounds and a pharmaceutically acceptable excipient, in the manufacture of a medicament for inhibiting KRAS (e.g., KRAS protein having a Q61H, G12C, G12D, G12V, G12S, G12A, G12R, or G13D mutation, or wild-type KRAS) in a subject in need thereof.

The present disclosure also provides a method comprising administering a therapeutically effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof to a subject (e.g., patient) (e.g., a subject in need thereof), thereby ameliorating, reducing, eliminating, ceasing, delaying the progression of, or improving one or more symptoms of the subject, such as one or more symptoms of a disease, disorder, or condition (e.g., a cancer). In some embodiments, the subject has a cancer characterized by a mutant KRAS (e.g., KRAS protein having a Q61H, G12C, G12D, G12V, G12S, G12A, G12R, or G13D mutation, or wild-type KRAS).

In some embodiments, administering a therapeutically effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, slows or prevents growth of a tumor. In some embodiments, administering a therapeutically effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, results in shrinkage of a tumor (e.g., tumor regression). In some embodiments, administering a therapeutically effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, results in at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% regression of a tumor, such as for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, administering a therapeutically effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, stabilizes a tumor. In some embodiments, administering a therapeutically effective amount of a compound provided herein (e.g., a compound of any one of Formulas I, IA, IB, IC, ID, IE, IF, IG, IH, IJ, IK, IL, IM, and IN), or a salt, ester, tautomer, prodrug, zwitterionic form, or stereoisomer thereof, stabilizes a tumor for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, the subject has a cancer characterized by a mutant KRAS (e.g., KRAS protein having a Q61H, G12C, G12D, G12V, G12S, G12A, G12R, or G13D mutation, or wild-type KRAS).

In some embodiments of any of the methods, uses, and medicaments provided herein, the disease, disorder, or condition is a cancer. In some embodiments of any of the methods, uses, and medicaments provided herein, the cancer is pancreatic cancer (e.g., pancreatic ductal adenocarcinoma), lung cancer (e.g., non-small cell lung cancer), colorectal cancer (CRC), endometrial cancer, uterine carcinosarcoma, Ewing sarcoma, osteosarcoma, Rhabdomyosarcoma, adrenocortical carcinoma, neuroblastoma, Wilm tumor, retinoblastoma, skin cancer, breast cancer, prostate cancer, head and neck cancer, or ovarian cancer. In some embodiments, the cancer is pancreatic cancer (e.g., pancreatic ductal adenocarcinoma), lung cancer (e.g., non-small cell lung cancer adenocarcinoma), or colorectal cancer (CRC). In some embodiments, the cancer is pancreatic cancer (e.g., pancreatic ductal adenocarcinoma). In some embodiments, the cancer is lung cancer (e.g., non-small cell lung cancer adenocarcinoma). In some embodiments, the cancer is colorectal cancer (CRC). In some embodiments, the cancer is or comprises a solid tumor.

In some embodiments of any of the methods, uses, and medicaments provided herein, the disease, disorder, or condition is related to KRAS, such as a disorder associated with a mutation of KRAS or dysregulation of KRAS. In some embodiments, the disease, disorder, or condition is related to the KRAS gene, such as a disease, disorder, or condition associated with a mutation of the KRAS gene or dysregulation of the KRAS gene. Mutation or dysregulation of KRAS or KRAS may include mutation or dysregulation of human K-Ras4a and/or human K-Ras4b. In some embodiments, the disease, disorder, or condition is related to the KRAS (e.g., human K-Ras4a or K-Ras4b) signaling pathway activity, such as a disease, disorder, or condition related to aberrant KRAS signaling pathway activity. In some embodiments, the disease, disorder, or condition is related to mutation or dysregulation of human K-Ras4b. In some embodiments, the disease, disorder, or condition is related to aberrant K-Ras4b signaling pathway activity. In some embodiments, the disease, disorder, or condition is related to mutation or dysregulation of human K-Ras4a. In some embodiments, the disease, disorder, or condition is related to aberrant K-Ras4a signaling pathway activity.

### Administration and Combination Therapy

The compounds provided herein (e.g., compounds of any one of Formulas I, IA, IB, IC, ID, IE, IF, IG, IH, IJ, TK, IL, IM, and IN) and forms thereof (e.g., salt, ester, tautomer, prodrug, zwitterionic form, stereoisomer, etc.), or compositions (e.g., pharmaceutical compositions) comprising the same, can be administered in various modes (e.g., orally, topically, or by injection). The amount of active ingredient (e.g., a compound provided herein in any suitable form thereof) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, IA, IB, IC, ID, IE, IF, IG, IH, IJ, IK, IL, IM, and IN), or a form thereof (e.g., salt, ester, tautomer, prodrug, zwitterionic form, stereoisomer, etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, IA, IB, IC, ID, IE, IF, IG, IH, IJ, IK, IL, IM, and IN), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, IA, IB, IC, ID, IE, IF, IG, IH, IJ, IK, IL, IM, and IN), or a form thereof, may be enhanced by administration of the compound, or a form thereof, and an additional agent (which may comprise an additional therapeutic regimen) that also provides a therapeutic benefit. For example, a compound provided herein (e.g., a compound of any one of Formulas I, IA, IB, IC, ID, IE, IF, IG, IH, IJ, IK, IL, IM, and IN), or a form thereof, may be administered in combination with an additional agent that may be effective in the treatment of a disease, disorder, or condition such as a cancer. Generally, the combination of a compound provided herein (e.g., a compound of any one of Formulas I, IA, IB, IC, ID, IE, IF, IG, IH, IJ, IK, IL, IM, and IN), or a form thereof, and one or more additional agents (e.g., therapeutic agents) may enhance the overall benefit experienced by the subject upon either component individually. In some embodiments, the effect may be additive. In some embodiments, the effect may be synergistic.

In some embodiments, a compound provided herein (e.g., a compound of any one of Formulas I, IA, IB, IC, ID, IE, IF, IG, IH, IJ, TK, IL, IM, and IN), or a form thereof (e.g., salt, ester, tautomer, prodrug, zwitterionic form, stereoisomer, etc.) is administered in combination with an anti-cancer agent (e.g., chemotherapeutic agent). An anti-cancer agent may be, for example, an alkylating agent, an antimitotic, a checkpoint inhibitor, an anti-metabolite, a plant alkaloid, a terpenoid, a cytotoxic agent, an antibiotic, a topoisomerase inhibitor, an aromatase inhibitor, an angiogenesis inhibitor, an anti-steroid, an anti-androgen, an mTOR inhibitor, monoclonal antibodies, or a tyrosine kinase inhibitor. An alkylating agent may be, for example, armustine, chlorambucil (LEUKERAN), cisplatin (PLATIN), carboplatin (PARAPLATIN), oxaliplatin (ELOXATIN), streptozocin (ZANOSAR), busulfan (MYLERAN), dacarbazine, ifosfamide, lomustine (CCNU), melphalan (ALKERAN), procarbazine (MATULAN), temozolomide (TEMODAR), thiotepa, or cyclophosphamide (ENDOXAN). An anti-metabolite may be, for example, cladribine (LEUSTATIN), mercaptopurine (PURINETHOL), thioguanine, pentostatin (NIPENT), cytosine arabinoside (cytarabine, ARA-C), gemcitabine (GEMZAR), fluorouracil (5-FU, CARAC), capecitabine (XELODA), leucovorin (FUSILEY), methotrexate (RHEUMATREX), or raltitrexed. An antimitotic may be, for example, a taxane such as docetaxel (TAXITERE) or paclitaxel (ABRAXANE, TAXOL), or a vinca alkaloid such as vincristine (ONCOVIN), vinblastine, vindesine, or vinorelbine (NAVELBINE). A checkpoint inhibitor may be an anti-PD-1 or anti-PD-L1 antibody such as pembrolizumab (KEYTRUDA), nivolumab (OPDIVO), MEDI4736, or MPDL3280A; anti-CTLA-4 antibody ipilimumab (YERVOY); or an agent that targets LAG3 (lymphocyte activation gene 3 protein), KIR (killer cell immunoglobulin-like receptor), 4-1BB (tumor necrosis factor receptor superfamily member 9), TIM3 (T-cell immunoglobulin and mucin-domain containing-3), or 0X40 (tumor necrosis factor receptor superfamily member 4). A topoisomerase inhibitor may be, for example, camptothecin (CTP), irinotecan (CAMPTOSAR), topotecan (HYCAMTIN), teniposide (VUMON), or etoposide (EPOSIN). A cytotoxic antibiotic may be, for example, actinomycin D (dactinomycin, COSMEGEN), bleomycin (BLENOXANE), doxorubicin (ADRIAMYCIN), daunorubicin (CERUBIDINE), epirubicin (ELLENCE), fludarabine (FLUDARA), idarubicin, mitomycin (MITOSOL), mitoxantrone (NOYANTRONE), or plicamycin. An aromatase inhibitor may be, for example, aminoglutethimide, anastrozole (ARIMIDEX), letrozole (FEMARA), vorozole (RIYIZOR), or exemestane (AROMASIN). An angiogenesis inhibitor may be, for example, genistein, sunitinib (SUTENT), or bevacizumab (AYASTIN). An anti-steroid or anti-androgen may be, for example, aminoglutethimide (CYTADREN), bicalutamide (CASODEX), cyproterone, flutamide (EULEXIN), or nilutamide (NILANDRON). A tyrosine kinase inhibitor may be, for example, imatinib (GLEEVEC), erlotinib (TARCEVA), afatinib (GILOTRIF), lapatinib (TYKERB), sorafenib (NEXAVAR), or axitinib (INLYTA). An mTOR inhibitor may be, for example, everolimus, temsirolimus (TORISEL), or sirolimus. Monoclonal antibody may be, for example, trastuzumab (HERCEPTIN) or rituximab (RITUXAN). Additional examples of agents that may be useful in combination with a compound provided herein, or an alternative form thereof, include, but are not limited to, amsacrine; Bacillus Calmette-Guerin (B—C-G) vaccine; buserelin (ETILAMIDE); chloroquine (ARALEN); clodronate, pamidronate, and other bisphosphonates; colchicine; demethoxyviridin; dichloroacetate; estramustine; filgrastim (NEUPOGEN); fludrocortisone (FLORINEF); goserelin (ZOLADEX); interferon; leucovorin; leuprolide (LUPRON); levamisole; lonidamine; mesna; metformin; mitotane (o,r′-DDD, LYSODREN); nocodazole; octreotide (SANDOSTATIN); perifosine; porfimer (particularly in combination with photo- and radiotherapy); suramin; tamoxifen; titanocene dichloride; tretinoin; anabolic steroids such as fluoxymesterone (HALOTESTIN); estrogens such as estradiol, diethylstilbestrol (DES), and dienestrol; progestins such as medroxyprogesterone acetate (MPA) and megestrol; and testosterone.

Two or more therapeutic agents, one of which is a compound provided herein (e.g., a compound of any one of Formulas I, IA, IB, IC, ID, IE, IF, IG, IH, IJ, IK, IL, IM, and IN) or a form thereof, may be administered in any order or may be administered simultaneously. If administered simultaneously, the multiple therapeutic agents may be provided in a single, unified form, or in multiple forms (such as, for example, as a single pill or as two separate pills). One of the therapeutic agents may be given in multiple doses, or both may be given as multiple doses. If not administered simultaneously, the timing between the multiple doses may be any duration of time ranging from a few minutes to four weeks.

Accordingly, in another aspect, the present disclosure provides a method for treating a disease, disorder, or condition (e.g., a cancer) in a subject (e.g., a human or animal subject) in need of such treatment comprising administering to the subject an amount of a compound provided herein (e.g., a compound of any one of Formulas I, IA, IB, IC, ID, IE, IF, IG, IH, IJ, IK, IL, IM, and IN), or a form thereof (e.g., salt, ester, tautomer, prodrug, zwitterionic form, stereoisomer, etc.), in combination with at least one additional agent for the treatment of the disease, disorder, or condition. In a related aspect, the present disclosure provides a composition (e.g., pharmaceutical composition) comprising a compound provided herein (e.g., a compound of any one of Formulas I, IA, IB, IC, ID, IE, IF, IG, IH, IJ, IK, IL, IM, and IN), or a form thereof (e.g., salt, ester, tautomer, prodrug, zwitterionic form, stereoisomer, etc.), and at least one additional agent for use in the treatment of a disease, disorder, or condition (e.g., a cancer).

In some embodiments, a method provided herein is used to treat a disease, disorder, or condition (e.g., a cancer) comprising administering to a subject in need thereof a therapeutically effective amount of a compound of any one of Formulas I, IA, IB, IC, ID, IE, IF, IG, IH, IJ, IK, IL, IM, and IN or a pharmaceutically acceptable salt thereof, wherein the disease, disorder, or condition is a cancer that has developed a resistance to one or more chemotherapeutic drugs and/or ionizing radiation. In some embodiments, a method provided herein is used to treat a disease, disorder, or condition (e.g., a cancer) comprising administering to a subject in need thereof a therapeutically effective amount of a compound of any one of Formulas I, IA, IB, IC, ID, IE, IF, IG, IH, IJ, IK, IL, IM, and IN or a pharmaceutically acceptable salt thereof, in combination with an additional agent, wherein the disease, disorder, or condition is a cancer that has developed a resistance to one or more chemotherapeutic drugs and/or ionizing radiation.

The compounds, compositions, and methods disclosed herein are useful for the treatment of a disease, disorder, or condition, such as a cancer. In certain embodiments, the disease is one of dysregulated cellular proliferation, including cancer. The cancer may be hormone-dependent or hormone-resistant, such as in the case of breast cancers. In certain embodiments, the cancer is or comprises a solid tumor. In other embodiments, the cancer is a lymphoma or leukemia. In certain embodiments, the cancer is a drug resistant phenotype of a cancer disclosed herein or otherwise known. Tumor invasion, tumor growth, tumor metastasis, and angiogenesis may also be treated using the compositions and methods disclosed herein. In some embodiments, the compounds, compositions, and methods provided herein are also useful in the treatment of precancerous neoplasias.

Cancers that may be treated by the methods disclosed herein include, but are not limited to, pancreatic cancer, colon cancer, rectal cancer, colorectal cancer, breast cancer, ovarian cancer, endometrial cancer, lung cancer, and prostate cancer; cancers of the oral cavity and pharynx (lip, tongue, mouth, larynx, pharynx), esophagus, stomach, small intestine, large intestine, colon, rectum, liver and biliary passages; pancreas, bone, connective tissue, skin, cervix, uterus, corpus endometrium, testis, bladder, kidney and other urinary tissues, including renal cell carcinoma (RCC); cancers of the eye, brain, spinal cord, and other components of the central and peripheral nervous systems, as well as associated structures such as the meninges; and thyroid and other endocrine glands. The term “cancer” also encompasses cancers that do not necessarily form solid tumors, including Hodgkin's disease, non-Hodgkin's lymphomas, multiple myeloma, and hematopoietic malignancies including leukemias (Chronic Lymphocytic Leukemia (CLL), Acute Lymphocytic Leukemia (ALL), Chronic Myelogenous Leukemia (CML), Acute Myelogenous Leukemia (AML),) and lymphomas including lymphocytic, granulocytic and monocytic lymphomas. Additional types of cancers which may be treated using the compounds and methods provided herein include, but are not limited to, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, head and neck cancer, hemangioblastoma, hepatocellular carcinoma, hepatoma, Kaposi's sarcoma, large cell carcinoma, leiomyosarcoma, leukemias, liposarcoma, lymphatic system cancer, lymphomas, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, oligodendroglioma, osteogenic sarcoma, epithelial ovarian cancer, papillary carcinoma, papillary adenocarcinomas, paraganglioma, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, non-small cell lung carcinoma, squamous cell carcinoma, sweat gland carcinoma, synovioma, thyroid cancer, uveal melanoma, and Wilm's tumor. Additional diseases and disorders that may be treated by the methods disclosed herein include, but are not limited to, diseases or disorders related to KRAS, such as diseases or disorders associated with a mutation of KRAS (e.g., KRAS protein having a Q61H, G12C, G12D, G12V, G12S, G12A, G12R, or G13D mutation, or wild-type KRAS) or dysregulation of KRAS, and diseases or disorders related to the KRAS gene, such as diseases or disorders associated with a mutation of the KRAS gene or dysregulation of the KRAS gene.

In some embodiments, the compounds, compositions, and methods provided herein are useful in the prevention and/or reduction of tumor invasion, growth, and/or metastasis.

The compounds, compositions, and methods provided herein may be useful in the treatment of humans as well as in the veterinary treatment of non-human animals including companion animals, exotic animals, and farm animals (e.g., as described herein), including mammals, rodents, and the like. For example, the compounds, compositions, and methods provided herein may be useful in the treatment of horses, dogs, or cats.

### Enumerated Embodiments

The following numbered embodiments, while non-limiting, are exemplary of certain aspects of the present disclosure:

1. A compound represented by Formula I:

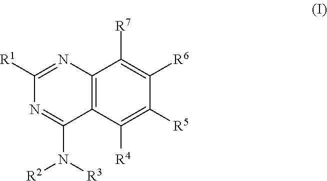

or a salt (e.g., pharmaceutically acceptable salt) thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen;
R^{S} is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R⁹;
each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹¹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R¹⁶ is independently selected from H, C₁₋₆alkyl, —C(O)C₁₋₆alkyl, and —C(O)(C₁₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
each R²⁰ is independently selected from —OH, —OC₁₋₆alkyl, —CN, —NH₂, —NHC₁₋₆alkyl, and halogen.

2. The compound of embodiment 1, wherein R⁶ is selected from:

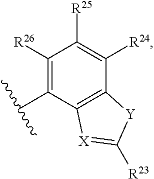

wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹.

3. The compound of embodiment 2, wherein X is C—CN and Y is S.

4. The compound of embodiment 2 or 3, wherein R²³ is —N(R¹²)₂.

5. The compound of any one of embodiments 2-4, wherein X is C—CN, Y is S, and R²³ is —NH₂.

6. The compound of any one of embodiments 1-5, wherein R⁶ is selected from:

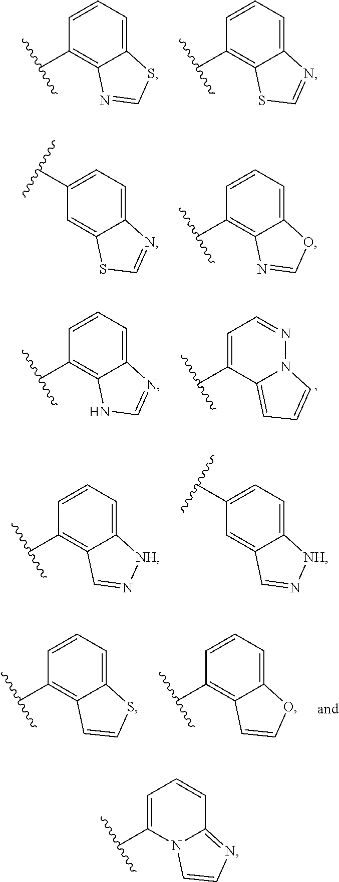

any of which is substituted with one or more R¹⁵.

7. The compound of any one of embodiments 1-6, wherein R⁶ is selected from:

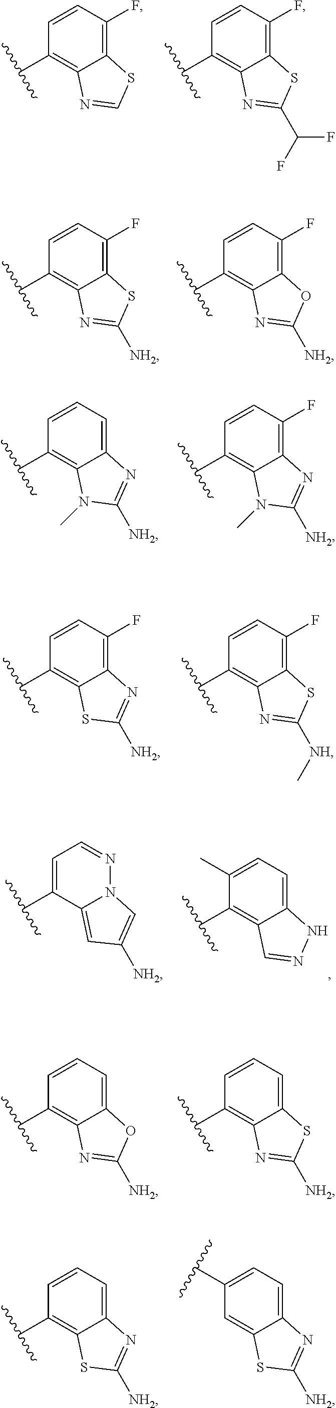

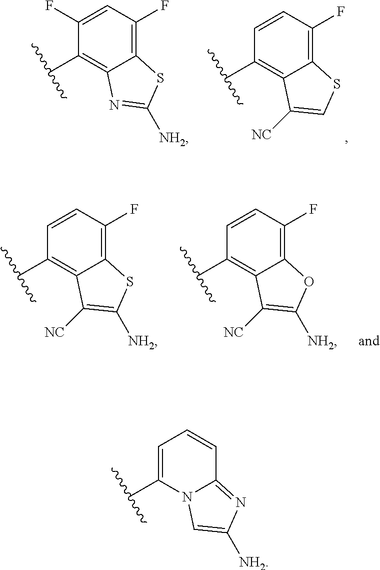 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

8. The compound of any one of embodiments 1-7, wherein R⁶ is selected from:

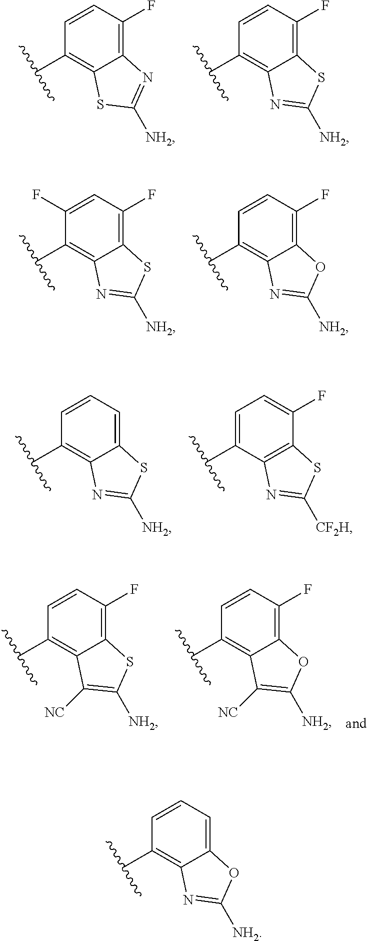

9. The compound of any one of embodiments 1-8, wherein the compound is a compound according to Formula IA:

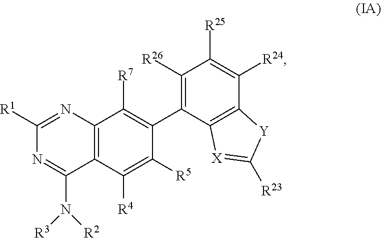

or a salt (e.g., pharmaceutically acceptable salt) thereof, wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹.

10. The compound of embodiment 9, wherein X is C—CN and Y is S.

11. The compound of embodiment 9 or 10, wherein R²³ is —N(R¹²)₂.

12. The compound of any one of embodiments 9-11, wherein X is C—CN, Y is S, R²³ is —N(R¹²)₂, and R²⁴ is a halogen (e.g., F).

13. The compound of any one of embodiments 1-12, wherein R² is selected from H and C₁₋₆alkyl, wherein any C₁₋₆ alkyl is unsubstituted or is substituted with one or more R¹³; and R³ is selected from a 4-6 membered heterocycle that is unsubstituted or substituted with one or more R¹⁰.

14. The compound of embodiment 13, wherein R³ is a 4-6 membered heterocycle that includes one or more heteroatoms selected from O, S, and N.

15. The compound of embodiment 14, wherein R³ is a 4-6 membered heterocycle that includes 1 heteroatom selected from O, S, and N.

16. The compound of embodiment 14 or 15, wherein R³ is a 4-6 membered heterocycle that includes a nitrogen atom.

17. The compound of embodiment 16, wherein R³ is an azetidine.

18. The compound of embodiment 16, wherein R³ is a pyrrolidine.

19. The compound of embodiment 16, wherein R³ is a piperidine.

20. The compound of any one of embodiments 16-19, wherein R³ includes an —NH— moiety.

21. The compound of any one of embodiments 13-20, wherein at least one R¹⁰ is selected from —OR¹² and a C₁₋₆alkyl substituted with —OH.

22. The compound of any one of embodiments 13-21, wherein at least one R¹⁰ is an unsubstituted C₁₋₆alkyl.

23. The compound of any one of embodiments 13-22, wherein each R¹⁰ is independently selected from —OR², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl that is unsubstituted or substituted with one or more R²⁰.

24. The compound of any one of embodiments 13-20, wherein R³ is unsubstituted.

25. The compound of any one of embodiments 1-24, wherein the compound is a compound according to Formula IB, IC, ID, or IE:

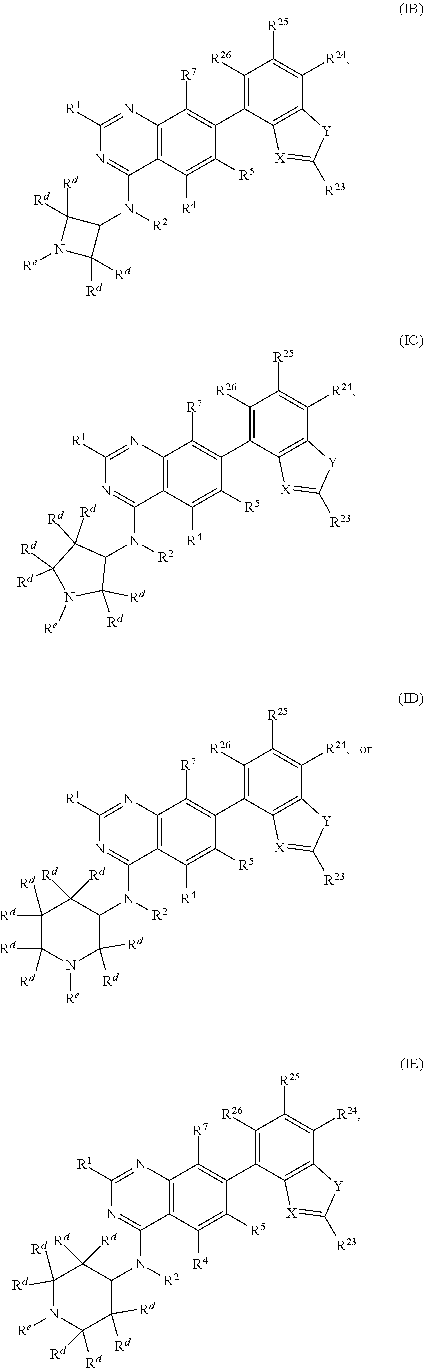

or a salt (e.g., pharmaceutically acceptable salt) thereof, wherein:
each R^{d} is independently selected from H, —OR¹², ═O, —C(O)(C₁₋₆alkylene)CN, —C(O)(C₁₋₆alkylene)OH, —C(O)(C₁₋₆alkyl), —C(O)N(R⁴)₂, —S(O)₂(C₁₋₆alkyl), halogen, and C₁₋₆alkyl, wherein any C₁₋₆alkyl is unsubstituted or substituted with one or more R²⁰;
R^{e} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹².

26. The compound of embodiment 25, wherein the compound is a compound according to Formula IB, or a salt (e.g., pharmaceutically acceptable salt) thereof.

27. The compound of embodiment 25, wherein the compound is a compound according to Formula IC, or a salt (e.g., pharmaceutically acceptable salt) thereof.

28. The compound of embodiment 25, wherein the compound is a compound according to Formula ID, or a salt (e.g., pharmaceutically acceptable salt) thereof.

29. The compound of embodiment 25, wherein the compound is a compound according to Formula IE, or a salt (e.g., pharmaceutically acceptable salt) thereof.

30. The compound of any one of embodiments 25-29, wherein X is C—CN and Y is S.

31. The compound of any one of embodiments 25-30, wherein R²³ is —N(R¹²)₂.

32. The compound of any one of embodiments 25-31, wherein X is C—CN, Y is S, R²³ is —N(R¹²)₂, and R²⁴ is a halogen (e.g., F).

33. The compound of any one of embodiments 25-32, wherein each R^{d} is H.

34. The compound of any one of embodiments 25-32, wherein at least one R^{d} is selected from —OR¹² and a C₁₋₆alkyl substituted with —OH.

35. The compound of any one of embodiments 25-32, wherein each R^{d} is independently selected from H, —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl that is unsubstituted or substituted with one or more R²⁰.

36. The compound of any one of embodiments 25-35, wherein R^{e} is H.

37. The compound of any one of embodiments 25-35, wherein R^{e} is C₁₋₆alkyl that is unsubstituted or substituted with one or more R²⁰.

38. The compound of embodiment 37, wherein R^{e} is C₁₋₆alkyl that is unsubstituted.

39. The compound of embodiment 37, wherein R^{e} is C₁₋₆alkyl that is substituted with one or more R²⁰, wherein each R²⁰ is independently selected from —OH, —OC₁₋₆alkyl, =O, and —CN.

40. The compound of any one of embodiments 13-39, wherein R² is H.

41. The compound of any one of embodiments 13-39, wherein R² is a C₁₋₆alkyl that is unsubstituted or is substituted with one or more R¹³.

42. The compound of embodiment 41, wherein R² is a C₁₋₂alkyl that is unsubstituted or is substituted with one or more R¹³.

43. The compound of embodiment 41 or 42, wherein R² is unsubstituted.

44. The compound of embodiment 41 or 42, wherein R² is substituted with one or more R¹³.

45. The compound of any one of embodiments 1-12, wherein R² and R³, together with the nitrogen atom to which they are attached, form a 4-9 membered heterocycle that is unsubstituted or is substituted with one or more R¹¹.

46. The compound of embodiment 45, wherein R² and R³, together with the nitrogen atom to which they are attached, form a 4-6 membered heterocycle that is unsubstituted or is substituted with one or more R¹¹.

47. The compound of embodiment 46, wherein the 4-6 membered heterocycle includes 1 or 2 heteroatoms selected from O, S, and N.

48. The compound of embodiment 47, wherein the 4-6 membered heterocycle includes 2 nitrogen atoms.

49. The compound of embodiment 48, wherein the 4-6 membered heterocycle is a piperazine.

50. The compound of any one of embodiments 46-49, wherein the 4-6 membered heterocycle includes an —NH— moiety.

51. The compound of embodiment 46 or 47, wherein the 4-6 membered heterocycle is an azetidine.

52. The compound of embodiment 46 or 47, wherein the 4-6 membered heterocycle is a pyrrolidine.

53. The compound of embodiment 46 or 47, wherein the 4-6 membered heterocycle is a piperidine.

54. The compound of any one of embodiments 46-53, wherein the 4-6 membered heterocycle is substituted with one or more R¹¹.

55. The compound of any one of embodiments 46-54, wherein at least one R¹¹ is selected from —OR¹² and a C₁₋₆alkyl substituted with —OH.

56. The compound of any one of embodiments 46-55, wherein at least one R¹¹ is an unsubstituted C₁₋₆alkyl.

57. The compound of any one of embodiments 46-53, wherein the 4-6 membered heterocycle is unsubstituted.

58. The compound of embodiment 45, wherein R² and R³, together with the nitrogen atom to which they are attached, form a bridged heterocycle that is unsubstituted or is substituted with one or more R¹¹.

59. The compound of embodiment 58, wherein R² and R³, together with the nitrogen atom to which they are attached, form a 7-9 membered bridged heterocycle that includes 1 or 2 heteroatoms selected from O, S, and N.

60. The compound of embodiment 59, wherein the bridged heterocycle includes 2 nitrogen atoms.

61. The compound of embodiment 60, wherein the bridged heterocycle is a bridged piperazine.

62. The compound of embodiment 60 or 61, wherein the bridged heterocycle includes an —NH-moiety.

63. The compound of embodiment 59, wherein the bridged heterocycle is a bridged piperidine.

64. The compound of any one of embodiments 58-63, wherein the bridged heterocycle is substituted with one or more R¹¹, wherein at least one R¹¹ is selected from —OR¹² and a C₁₋₆alkyl substituted with —OH.

65. The compound of any one of embodiments 58-64, wherein the bridged heterocycle is substituted with one or more R¹¹, wherein at least one R¹¹ is an unsubstituted C₁₋₆alkyl.

66. The compound of any one of embodiments 58-63, wherein the bridged heterocycle is unsubstituted.

67. The compound of embodiment 45, wherein R² and R³, together with the nitrogen atom to which they are attached, form a spirocycle that is unsubstituted or is substituted with one or more R¹¹.

68. The compound of embodiment 67, wherein the spirocycle includes an —NH— moiety.

69. The compound of any one of embodiments 45-68, wherein R² and R³, together with the nitrogen atom to which they are attached, form a 4-9 membered heterocycle that is unsubstituted or is substituted with one or more R¹¹, wherein the one or more R¹¹ are independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.

70. The compound of any one of embodiments 45-68, wherein R² and R³, together with the nitrogen atom to which they are attached, form a 4-9 membered heterocycle that is unsubstituted or is substituted with one or more R¹¹, wherein the heterocycle includes an —NH— moiety, and wherein the one or more R¹¹ are independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.

71. The compound of any one of embodiments 1-12 and 45-70, wherein the compound is a compound according to Formula IF:

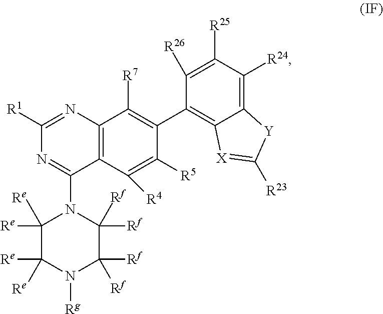

or a salt (e.g., pharmaceutically acceptable salt) thereof, wherein:
each R^{e} and R^{f} is independently selected from R¹¹ and hydrogen, wherein an R^{e} and an R^{f} can optionally join together to form a 4-6 membered ring, or a first R^{f} and a second R^{f} connected to adjacent atoms can optionally join together to form a 3-5 membered ring, or a first R^{e} and a second R^{e} connected to adjacent atoms can optionally join together to form a 3-5 membered ring;
R^{g} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

72. The compound of embodiment 71, wherein X is C—CN and Y is S.

73. The compound of embodiment 71 or 72, wherein R²³ is —N(R¹²)₂.

74. The compound of any one of embodiments 71-73, wherein X is C—CN, Y is S, R²³ is —N(R¹²)₂, and R²⁴ is a halogen (e.g., F).

75. The compound of any one of embodiments 71-74, wherein R^{g} is selected from H and C₁₋₆alkyl that is unsubstituted.

76. The compound of embodiment 75, wherein R^{g} is H.

77. The compound of embodiment 75, wherein R^{g} is C₁₋₆alkyl that is unsubstituted.

78. The compound of any one of embodiments 71-77, wherein each R^{e} and R^{f} is independently selected from R¹¹ and hydrogen.

79. The compound of embodiment 78, wherein each R^{e} and R^{f} is independently selected from hydrogen, —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R⁴)C(O)(C₁₋₆alkyl), —N(R¹⁴)C(O)(C₁₋₆alkylene)OR⁴, halogen, and C₁₋₆alkyl, wherein any C₁₋₆alkyl is unsubstituted or substituted with one or more R²⁰.

80. The compound of embodiment 78 or 79, wherein at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN).

81. The compound of embodiment 78 or 79, wherein each R^{e} and R^{f} is hydrogen.

82. The compound of any one of embodiments 71-76, wherein each R^{e}, R^{f}, and R^{g} is H.

83. The compound of any one of embodiments 71-77, wherein an R^{o} and an Rejoin together to form a 4-6 membered ring.

84. The compound of any one of embodiments 71-77, wherein a first R^{f} and a second R^{f} connected to adjacent atoms join together to form a 3-5 membered ring, or a first R^{e} and a second R^{e} connected to adjacent atoms join together to form a 3-5 membered ring.

85. The compound of any one of embodiments 1-12 and 45-84, wherein the compound is a compound according to Formula IG, IH, IJ, IK, IL, IM, or IN:

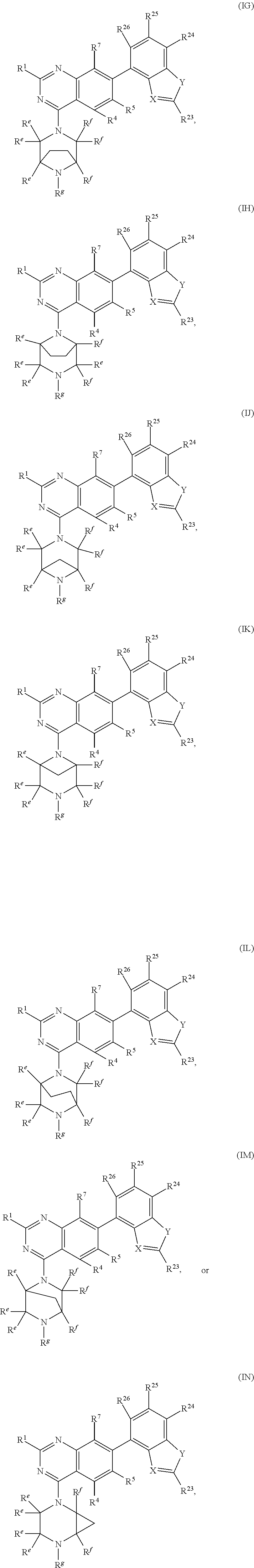

or a salt (e.g., pharmaceutically acceptable salt) thereof, wherein:
each R^{e} and R^{f} is independently selected from R¹¹ and hydrogen;
R^{g} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

86. The compound of embodiment 85, wherein the compound is a compound of Formula IG or a salt (e.g., a pharmaceutically acceptable salt) thereof.

87. The compound of embodiment 85, wherein the compound is a compound of Formula IH or a salt (e.g., a pharmaceutically acceptable salt) thereof.

88. The compound of embodiment 85, wherein the compound is a compound of Formula IJ or a salt (e.g., a pharmaceutically acceptable salt) thereof.

89. The compound of embodiment 85, wherein the compound is a compound of Formula IK or a salt (e.g., a pharmaceutically acceptable salt) thereof.

90. The compound of embodiment 85, wherein the compound is a compound of Formula IL or a salt (e.g., a pharmaceutically acceptable salt) thereof.

91. The compound of embodiment 85, wherein the compound is a compound of Formula IM or a salt (e.g., a pharmaceutically acceptable salt) thereof.

92. The compound of embodiment 85, wherein the compound is a compound of Formula IN or a salt (e.g., a pharmaceutically acceptable salt) thereof.

93. The compound of any one of embodiments 85-92, wherein R^{g} is selected from H and C₁₋₆alkyl that is unsubstituted.

94. The compound of embodiment 93, wherein R^{g} is H.

95. The compound of embodiment 93, wherein R^{g} is C₁₋₆alkyl that is unsubstituted.

96. The compound of any one of embodiments 85-95, wherein each R^{e} and R^{f} is independently selected from hydrogen, —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.

97. The compound of embodiment 96, wherein at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN).

98. The compound of any one of embodiments 85-97, wherein each R^{e} and R^{f} is hydrogen.

99. The compound of any one of embodiments 85-94, wherein each R^{e}, R^{f}, and R^{g} is H.

100. The compound of any one of embodiments 1-99, wherein R⁴ is H.

101. The compound of any one of embodiments 1-99, wherein R⁴ is —OR¹⁴.

102. The compound of embodiment 101, wherein R⁴ is —OCH₃.

103. The compound of any one of embodiments 1-102, wherein R⁵ is a halogen (e.g., F or Cl).

104. The compound of any one of embodiments 1-102, wherein R⁵ is selected from C₁₋₆alkyl that is unsubstituted or substituted with one or more R¹³.

105. The compound of embodiment 104, wherein R⁵ is selected from C₁₋₆alkyl that is substituted with one or more halogens or —CN.

106. The compound of embodiment 105, wherein R⁵ is selected from —CF₂H, —CF₃, —CH₂CN, and —CH₂CH₃.

107. The compound of embodiment 106, wherein R⁵ is —CF₃.

108. The compound of any one of embodiments 1-102, wherein R⁵ is —CN or —OR¹².

109. The compound of any one of embodiments 1-102, wherein R⁵ is a 3-6 membered heterocycle.

110. The compound of any one of embodiments 1-102, wherein R⁵ is a 5-6 membered heteroaryl.

111. The compound of any one of embodiments 1-110, wherein R⁷ is F.

112. The compound of any one of embodiments 1-111, wherein R¹ is H.

113. The compound of any one of embodiments 1-111, wherein R¹ is C₁₋₆alkyl that is unsubstituted or substituted with one or more R¹³.

114. The compound of embodiment 113, wherein R¹ is C₁₋₃alkyl that is unsubstituted or substituted with one or more R¹.

115. The compound of embodiment 114, wherein R¹ is C₁₋₃alkyl that is unsubstituted.

116. The compound of any one of embodiments 1-111, wherein R¹ is —OR⁸ or —O(C₁₋₆alkylene)R⁸, wherein any C₁₋₆alkylene is unsubstituted or substituted with one or more R¹³.

117. The compound of embodiment 116, wherein R¹ is —OR⁸.

118. The compound of embodiment 116, wherein R¹ is —O(C₁₋₆alkylene)R⁸.

119. The compound of embodiment 118, wherein R¹ is —O(C₁₋₆alkylene)R⁸, wherein any C₁₋₆alkylene is unsubstituted.

120. The compound of any one of embodiments 116-119, wherein R⁸ is H.

121. The compound of any one of embodiments 116-119, wherein R⁸ is C₁₋₆alkyl that is unsubstituted or substituted with one or more R¹³.

122. The compound of embodiment 121, wherein R⁸ is C₁₋₃alkyl that is unsubstituted or substituted with one or more R¹.

123. The compound of embodiment 122, wherein R⁸ is C₁₋₃alkyl that is unsubstituted.

124. The compound of any one of embodiments 121-123, wherein R¹ is selected from

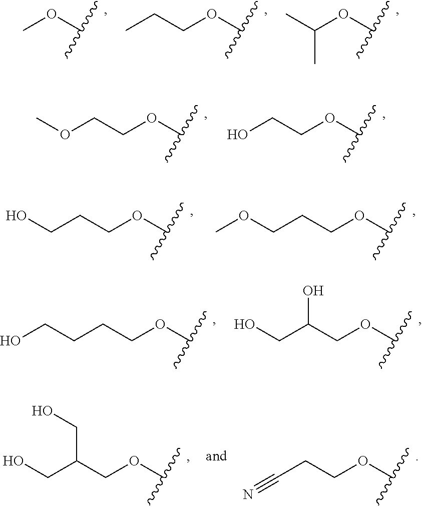

125. The compound of any one of embodiments 116-119, wherein R⁸ is a heteroaryl that comprises 3-8 members and is unsubstituted or substituted with one or more R¹⁹.

126. The compound of embodiment 125, wherein R⁸ is a heteroaryl that comprises 3-8 members including one or more heteroatoms selected from N, O, and S.

127. The compound of embodiment 126, wherein R⁸ is a heteroaryl that comprises 3-8 members including two or more heteroatoms selected from N, O, and S.

128. The compound of embodiment 127, wherein R⁸ is a heteroaryl that comprises 3-8 members including two nitrogen atoms.

129. The compound of any one of embodiments 125-128, wherein R⁸ is a heteroaryl that is unsubstituted.

130. The compound of any one of embodiments 125-129, wherein R¹ is

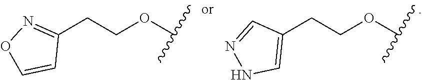 [SMILES: *OCCc1ccon1 ; *OCCc1cn[nH]c1]

131. The compound of any one of embodiments 116-119, wherein R⁸ is a heterocycle that comprises 3-8 members and is unsubstituted or substituted with one or more R¹⁹.

132. The compound of embodiment 131, wherein R⁸ is a heterocycle that comprises 3-8 members including one or more heteroatoms selected from N, O, and S.

133. The compound of embodiment 132, wherein R⁸ is a heterocycle that comprises 3-8 members including two or more heteroatoms selected from N, O, and S.

134. The compound of embodiment 133, wherein R⁸ is a heterocycle that comprises 3-8 members including three or more heteroatoms selected from N, O, and S.

135. The compound of embodiment 133, wherein R⁸ is a heterocycle that comprises 3-8 members including two oxygen atoms.

136. The compound of any one of embodiments 131-136, wherein R⁸ is a heterocycle that is unsubstituted.

137. The compound of any one of embodiments 131-136, wherein R¹ is selected from

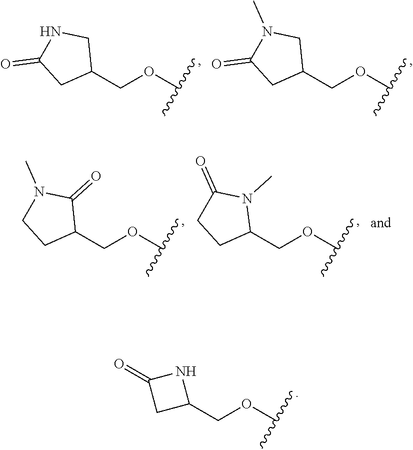

138. The compound of any one of embodiments 131-136, wherein R¹ is selected from

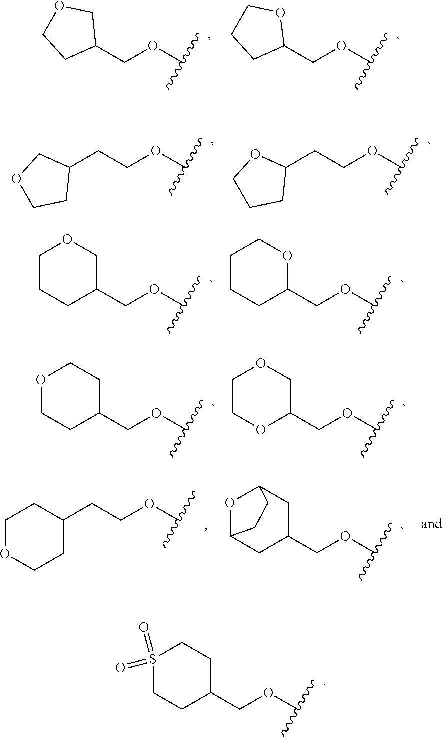

139. The compound of any one of embodiments 131-136, wherein R¹ is not a structure selected from:

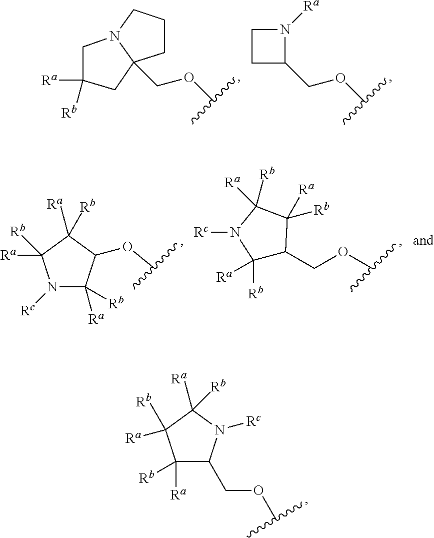

wherein each R^{a} and R^{b} are each independently selected from halogen, C₁₋₆alkyl, —OR¹², and H, wherein any C₁₋₆alkyl is unsubstituted or is substituted with one or more R¹³; and R^{e} is selected from C₁₋₆ alkyl that is unsubstituted or is substituted with one or more R¹³.

140. The compound of any one of embodiments 131-136, wherein R¹ is not a structure selected from:

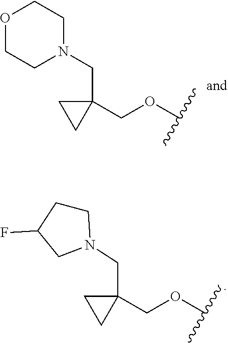 [SMILES: *OCC1(CN2CCOCC2)CC1 ; *OCC1(CN2CCC(F)C2)CC1]

141. The compound of any one of embodiments 116-119, wherein R⁸ is an aryl that comprises 3-8 members and is unsubstituted or substituted with one or more R¹⁹.

142. The compound of embodiment 141, wherein R⁸ is phenyl.

143. The compound of embodiment 141 or 142, wherein R⁸ is an aryl that is unsubstituted.

144. The compound of any one of embodiments 141-143, wherein R¹ is

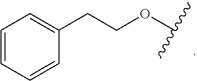 [SMILES: *OCCc1ccccc1]

145. The compound of any one of embodiments 116-119, wherein R⁸ is a carbocycle that comprises 3-8 members and is unsubstituted or substituted with one or more R⁹.

146. The compound of embodiment 145, wherein R⁸ is a carbocycle that comprises 3-6 members.

147. The compound of embodiment 145 or 146, wherein R⁸ is a carbocycle that is unsubstituted.

148. The compound of any one of embodiments 145-147, wherein R¹ is selected from

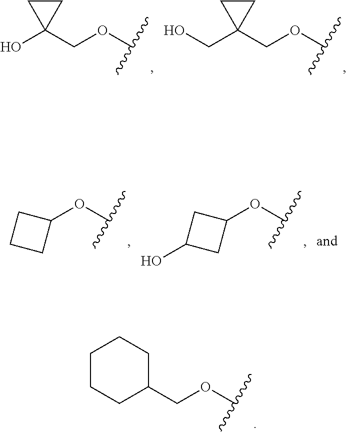 [SMILES: *OCC1(O)CC1 ; *OCC1(CO)CC1 ; *OC1CCC1 ; *OC1CC(O)C1 ; *OCC1CCCCC1]

149. The compound of any one of embodiments 1-111, wherein R¹ is —N(R¹⁶)₂.

150. The compound of embodiment 149, wherein R¹ is —NHR¹⁶.

151. The compound of embodiment 149 or 150, wherein each R¹⁶ is independently selected from C₁₋₆alkyl that is unsubstituted or substituted with one or more R¹³.

152. The compound of embodiment 149 or 150, wherein each R¹⁶ is independently selected from C₁₋₆alkyl and —C(O)C₁₋₆alkyl, wherein any C₁₋₆alkyl is unsubstituted or substituted with one or more R¹³.

153. The compound of any one of embodiments 149-152, wherein R¹ is selected from

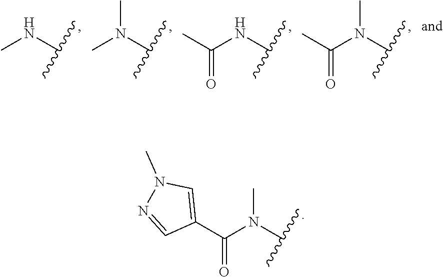

154. The compound of any one of embodiments 1-111, wherein R¹ is a 4-7 membered nitrogen-containing heterocycle that is unsubstituted or substituted with one or more R¹⁷.

155. The compound of embodiment 154, wherein R¹ is a 4-5 membered nitrogen-containing heterocycle.

156. The compound of embodiment 154 or 155, wherein R¹ is a nitrogen-containing heterocycle that is unsubstituted.

157. The compound of any one of embodiments 154-156, wherein R¹ is selected from

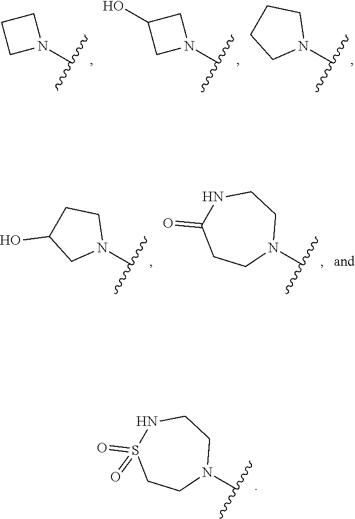

158. A compound shown in Table 2, or a salt (e.g., pharmaceutically acceptable salt) thereof.

159. A pharmaceutical composition comprising a compound of any one of embodiments 1-158, or a salt (e.g., pharmaceutically acceptable salt) thereof, and a pharmaceutically acceptable excipient.

160. A compound of any one of embodiments 1-158, or a salt (e.g., pharmaceutically acceptable salt) thereof, for use as a medicament.

161. The compound of embodiment 160, wherein the medicament is useful in the prevention or treatment of a disease, disorder, or condition ameliorated by the inhibition of KRAS having a G12D mutation.

162. The compound of embodiment 160 or 161, wherein the medicament is useful in the prevention or treatment of a cancer.

163. The compound of embodiment 162, wherein the cancer is selected from the group consisting of pancreatic cancer, colorectal cancer, and lung cancer.

164. A compound of any one of embodiments 1-158, or a salt (e.g., pharmaceutically acceptable salt) thereof, for use in the treatment of a disease, disorder, or condition.

165. The compound of embodiment 164, wherein the disease, disorder, or condition is a cancer.

166. The compound of embodiment 165, wherein the cancer is selected from the group consisting of pancreatic cancer, colorectal cancer, and lung cancer.

167. The compound of any one of embodiments 164-166, wherein the compound is used in the treatment of a disease, disorder, or condition in a subject in need thereof.

168. A compound of any one of embodiments 1-158, or a salt (e.g., pharmaceutically acceptable salt) thereof, for use in the manufacture of a medicament.

169. The compound of embodiment 168, wherein the medicament is useful in the prevention or treatment of a disease, disorder, or condition ameliorated by the inhibition of KRAS having a G12D mutation.

170. The compound of embodiment 168 or 169, wherein the medicament is useful in the treatment of a cancer.

171. The compound of embodiment 170, wherein the cancer is selected from the group consisting of pancreatic cancer, colorectal cancer, and lung cancer.

172. A method, comprising administering a therapeutically effective amount of a compound of any one of embodiments 1-158, or a salt (e.g., pharmaceutically acceptable salt) thereof, to a subject in need thereof.

173. The method of embodiment 172, wherein the subject has a disease, disorder, or condition ameliorated by the inhibition of KRAS having a G12D mutation.

174. The method of embodiment 172 or 173, wherein the subject has a cancer.

175. The method of embodiment 174, wherein the subject was previously diagnosed with the cancer.

176. The method of embodiment 174, wherein the subject has previously undergone a treatment regimen for the cancer.

177. The method of embodiment 176, wherein the subject has previously entered remission from the cancer.

178. The method of any one of embodiments 174-177, wherein the cancer is selected from the group consisting of pancreatic cancer, colorectal cancer, and lung cancer.

179. The method of any one of embodiments 172-178, wherein the compound, or the salt thereof, is administered in combination with an additional therapeutic agent.

180. The use of a compound of any one of embodiments 1-158, or a salt (e.g., pharmaceutically acceptable salt) thereof, for the manufacture of a medicament for the treatment of a cancer.

181. The use of embodiment 180, wherein the cancer is selected from the group consisting of pancreatic cancer, colorectal cancer, and lung cancer.

182. A method, comprising contacting a KRAS protein with a compound of any one of embodiments 1-158, or a salt (e.g., pharmaceutically acceptable salt) thereof.

183. The method of embodiment 182, wherein contacting the KRAS protein with the compound modulates KRAS.

184. The method of embodiment 182 or 183, wherein the KRAS protein has a G12D mutation.

185. The method of any one of embodiments 182-184, wherein the KRAS protein is in an active (GTP-bound) state.

186. The method of any one of embodiments 182-184, wherein the KRAS protein is in an inactive (GDP-bound) state.

187. The method of any one of embodiments 182-186, wherein the KRAS protein is located within a cell.

188. The method of embodiment 187, wherein the cell is located within a subject.

189. The method of embodiment 188, wherein the subject is a human.

190. The method of embodiment 188 or 189, wherein the subject has a cancer.

191. The method of embodiment 190, wherein the cancer is selected from the group consisting of pancreatic cancer, colorectal cancer, and lung cancer.

192. A method of inhibiting the function of a KRAS protein having a G12D mutation, comprising contacting the KRAS protein with a compound of any one of embodiments 1-158, or a salt (e.g., pharmaceutically acceptable salt) thereof.

193. The method of embodiment 192, wherein the KRAS protein has a G12D mutation.

194. The method of embodiment 192 or 193, wherein the KRAS protein is in an active (GTP-bound) state.

195. The method of embodiment 192 or 193, wherein the KRAS protein is in an inactive (GDP-bound) state.

196. The method of any one of embodiments 192-195, wherein the KRAS protein is located within a cell.

197. The method of embodiment 196, wherein the cell is located within a subject.

198. The method of embodiment 197, wherein the subject is a human.

199. The method of embodiment 197 or 198, wherein the subject has a cancer.

200. The method of embodiment 199, wherein the cancer is selected from the group consisting of pancreatic cancer, colorectal cancer, and lung cancer.

201. A compound capable of inhibiting a KRAS protein having a G12D mutation in both its active (GTP-bound) and inactive (GDP-bound) state.

202. The compound of embodiment 201, wherein the compound:
(i) has IC₅₀≤0.1 μM, 0.1 M<IC₅₀≤1 μM, or 1 μM<IC₅₀≤10 μM in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF1 RBD-3×FLAG (52-151); 50 nM Avi-KRAS G12R (amino acids 1-169) GppNHp/RAF1 RBD-3×FLAG (52-151); 50 nM Avi-KRAS G12V (amino acids 1-169) GppNHp/RAF1 RBD-3×FLAG (52-151); 50 nM Avi-KRAS WT (amino acids 1-169) GppNHp/RAF1 RBD-3×FLAG (52-151); and/or 75 nM Avi-RAF1 RBD-3×FLAG); and/or
(ii) has IC₅₀≤0.1 μM or 0.1 μM<IC₅₀≤1 μM in the assay of Biological Example 2 (e.g., cell-based pERK HTRF assay in GP2d (G12D) cell; and/or
(iii) has IC₅₀≤0.1 μM, 0.1 μM<IC₅₀≤1 μM, 1 μM<IC₅₀≤10 μM, or IC₅₀>10 μM in the assay of Biological Example 3 (e.g., cell-based pERK HTRF assay in SW620 (G12V) cell).

203. The compound of embodiment 202, wherein the compound:
(i) has IC₅₀≤0.1 μM or 0.1 μM<IC₅₀≤1 μM in the assay of Biological Example 1; and/or
(ii) has IC₅₀≤0.1 μM or 0.1 μM<IC₅₀≤1 μM in the assay of Biological Example 2.

204. The compound of any one of embodiments 201-203, wherein the compound is capable of irreversibly binding the KRAS protein.

205. The compound of any one of embodiments 201-203, wherein the compound is capable of reversibly binding the KRAS protein.

206. The compound of any one of embodiments 201-205, wherein the compound is a compound according to any one of embodiments 1-158.

### EXAMPLES

Selected abbreviations used in the preceding sections and the Examples are summarized in Table 1.

TABLE 1
Abbreviations.

| Abbreviation | Term |
|---|---|
| MeCN | acetonitrile |
| cm | centimeter |
| ° C. | degrees Celsius |
| ° K | degrees Kelvin |
| BINAP | [1,1′-Binaphthalene]-2,2′- |
| | diyl)bis(diphenylphosphane) |
| DCM | dichloromethane |
| DIEA | N,N-diisopropylethylamine (Hunig's base) |
| DMF | dimethylformamide |
| DMSO | dimethyl sulfoxide |
| DPPA | diphenylphosphoryl azide |
| eq | equivalents |
| ESI | electrospray ion-mass spectrometry |
| EtOH | ethanol |
| EtOAc | ethyl acetate |
| g | gram |
| Hz | hertz |
| HATU | Hexafluorophosphate azabenzotriazole tetramethyl uronium |
| HPLC | high performance liquid chromatography |
| HTRF | homogenous time-resolved fluorescence |
| h | hour |
| kDa | kilodalton |
| LC | liquid chromatography |
| LCMS | liquid chromatography-mass spectrometry |
| L | liter |
| MS | mass spectra |
| MHz | megahertz |
| MeOH | methanol |
| MTBE | methyl tert-butyl ether |
| μg | microgram |
| μL | microliter |
| μM | micromolar |
| μm | micron |
| μs | microsecond |
| mg | milligram |
| mL | milliliter |
| mm | millimeter |
| mM | millimolar |
| mmol | millimole |
| min | minute |
| M | molar |
| nL | nanoliter |
| nm | nanometer |
| NCS | N-chlorosuccinimide |
| NMP | N-methyl-2-pyrrolidone |
| PE:EA | Petroleum ether:ethyl acetate |
| ppm | parts per million |
| PTLC | preparative thin layer chromatography |
| ¹HNMR | proton nuclear magnetic resonance |
| RBD | receptor binding domain |
| RP | reverse phase |
| rpm | revolutions per minute |
| SPR | surface plasmon resonance |
| THF | tetrahydrofuran |
| TMS | tetramethylsilane |
| TLC | thin layer chromatography |
| SOCl₂ | thionyl chloride |
| TEA | triethylamine |
| TFA | trifluoroacetic acid |
| TCEP | tris(2-carboxyethyl)phosphine |
| UV | ultraviolet |
| UV/Vis | ultraviolet/visible |

### Materials and Methods

Preparative thin layer chromatography (PTLC) separations described herein were typically performed on 20×20 cm plates (500-μm thick silica gel).

Chromatographic purifications were typically performed using Biotage Isolera One automated system running Biotage Isolera One 2.0.6 software (Biotage LLC, Charlotte, NC). Flow rates were the default values specified for the column in use. Reverse phase chromatography was performed using elution gradients of water and acetonitrile on KP-C18-HS Flash+ columns (Biotage LLC) of various sizes. Typical loading was between 1:50 and 1:1000 crude sample: RP SiO₂ by weight. Normal phase chromatography was performed using elution gradients of various solvents (e.g., hexane, ethyl acetate, methylene chloride, methanol, acetone, chloroform, MTBE, etc.). The columns were SNAP Cartridges containing KP-SIL or SNAP Ultra (25 pm spherical particles) of various sizes (Biotage LLC). Typical loading was between 1:10 to 1:150 crude sample: SiO₂ by weight. Alternatively, silica gel chromatography was performed on a Biotage Horizon flash chromatography system.

¹HNMR analyses of intermediates and exemplified compounds were typically performed on a Bruker Ascend™ 400 spectrometer (operating at 400 MHz), Bruker Ascend 700 MHz Advance Neo Spectrometer (Bruker-Biospin) or Bruker Advance ultrashield 300/54 (operating at 300 MHz) at 298° K following standard operating procedure suggested by manufacturer. Reference frequency was set using TMS as an internal standard. Chemical shift values (6) are reported in parts per million (ppm) with splitting patterns abbreviated to: s (singlet), br. s (broad singlet), d (doublet), dd (double doublet), t (triplet), and m (multiplet). The coupling constant (J) is given in Hz. Typical deuterated solvents were utilized as indicated in the individual examples.

LCMS analysis were typically performed using one of the following conditions:
(1) LCMS spectra were taken on an Agilent Technologies 6120B Quadrupole spectrometer. The mobile phase for the LC was acetonitrile (A) with 0.1% formic acid, and water (B) with 0.1% formic acid, and the eluent gradient was from 5-95% A in 6.0 min, 5%-40% A in 6.0 min, 80-100% A in 6.0 min using a poroshell 120 EC-C18 50 mm×3.0 mm×2.7 μm capillary column; Flow Rate: 0.7 mL/min. Mass spectra (MS) were measured by electrospray ion-mass spectroscopy (ESI). All temperatures are in degrees Celsius (° C.) unless otherwise noted.
(2) LCMS spectra were taken on an Agilent Technologies 1290-6420 Triple Quadrupole spectrometer: The mobile phase for the LC was acetonitrile (A) with 0.05% formic acid, and water (B) with 0.05% formic acid, and the eluent gradient was from 5-95% A in 5.0 min, using a ZORBAX SB—C18 50 mm×2.1 mm×1.8 μm capillary column; Flow Rate: 0.3 mL/min. Mass spectra (MS) were measured by electrospray ion-mass spectroscopy (ESI). All temperatures are in degrees Celsius unless otherwise noted.
(3) LC-MS analysis was performed using an Agilent 6120b single quadrupole mass spectrometer with an Agilent 1260 infinity II chromatography separations module and Agilent 1260 infinity II photodiode array detector controlled by Agilent Chemstation software. The HPLC column used was an Agilent ZORBAX Eclipse XDB-C18 4.6 mm×150 mm×3.5 m RapidResol column with a mobile phase of water (0.1% formic acid)/MeCN (0.1% formic acid) and a gradient of 5-95% MeCN over 10 minutes at a flow rate of 1 mL/min. Accurate mass data was obtained using a Thermo Fisher extractive plus EMR orbitrap LCMS system. Exact mass values were calculated by ChemCalc.
(4) LCMS spectra were taken on an alliance Waters 2695 coupled to a dual absorbance detector waters 2487 and a waters micro mass ZQ-2000 single quadrupole spectrometer. The mobile phase for the LC was acetonitrile (A) and water (B) with 0.01% formic acid, and the eluent gradient was from 5-100% A in 10.0 minute using a Kromasil 100-5-C18 150 mm×4.6 mm×5 μm column. Mass spectra (MS) were measured by electrospray ion-mass spectroscopy (ESI). All temperatures are in degrees Celsius unless otherwise noted.

Typically, analytical HPLC mass spectrometry conditions were as follows:
LC1: Agilent Technologies 1260 Infinity coupled, Column: poroshell 120 EC-C18 150 mm×4.6 mm×4 m; Temperature: 40° C.; Fluent: 5:95 v/v acetonitrile/water+0.02% trifluoroacetic acid in 20 min; Flow Rate: 1.2 mL/min; Detection: VWD, 190-600 nm.
LC2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μm OBD™ 19×250 mm column with a mobile phase of water/MeCN or water (0.1% TFA)/MeCN (0.1% TFA).

Preparative HPLC were carried out with one of the following two conditions:
Condition 1: GILSON Preparative HPLC System; Column: Ultimate XB—C18, 21.2 mm×250 mm, 5 μm; Mobile phase: Water with 0.1% trifluoroacetic acid; MeCN with 0.10% trifluoroacetic acid; Method: 15 minutes gradient elution; Initial organic: 10% to 30%; Final organic: 60% to 80%; UV1: 240; UV2: 230; Flow: 15 mL/min.
Condition 2: C18-Reverse phase preparative HPLC was performed using a Waters purification system with 2489 UV/Vis detector, 2545 Gradient module, and Fraction collector III controlled by Waters Chromescope v1.6. The preparative HPLC column used was a Waters XBridge® Prep C18 5 μm OBD™ 19×250 mm column with a mobile phase of water/MeCN or water (0.1% TFA)/MeCN (0.1% TFA).

Compound names were generated with ChemDraw Professional.

The compounds provided herein (e.g., compounds in Table 2 or Table 3), including in various forms such as salts, esters, tautomers, prodrugs, zwitterionic forms, stereoisomers, etc., may be prepared according to various methods including those set forth in the following Examples or in International Patent Application No. PCT/US2022/016487, filed Feb. 15, 2022, and published as WO2022/177917, which is herein incorporated by reference in its entirety.

A compound of present disclosure, such as a compound of a formula included in Table 2 or Table 3, may be synthesized according to one of the general routes outlined in International Patent Application No. PCT/US2022/016487, filed Feb. 15, 2022, and published as WO2022/177917, or by various other methods generally known in the art.

### Synthetic Example 1: Synthesis of 4-(4-((1R,5S)-3,8-diazabicyclo[3.2.1]octan-3-yl)-8-fluoro-6-(trifluoromethyl)quinazolin-7-yl)-2-amino-7-fluorobenzo[b]thiophene-3-carbonitrile (Compound 5)

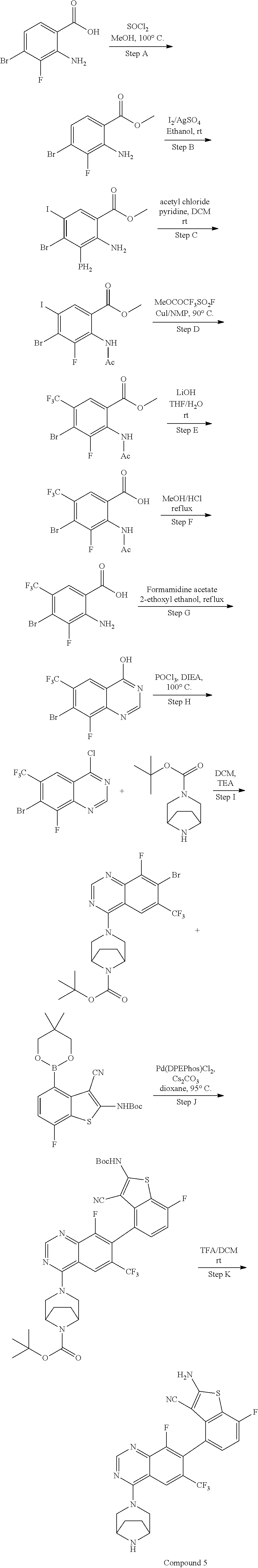

Step A: Preparation of methyl 2-amino-4-bromo-3-fluorobenzoate: To a stirring solution of 2-amino-4-bromo-3-fluorobenzoic acid (5.0 g, 21.4 mmol) in MeOH (30 mL) was added dropwise thionyl chloride (15.6 ml, 21 mmol) at 0° C. under argon. The resulting mixture was heated to 100° C. for 16 hours. The solvent was evaporated, and the residue was dissolved in ethyl acetate (100 mL). The organic layer was washed with a saturated aqueous NaHCO₃ solution then dried over Na₂SO₄, filtered, and concentrated under vacuum. The resulting crude material was purified by silica gel column chromatography using EtOAc in hexanes (0% to 20%) as eluent to give methyl 2-amino-4-bromo-3-fluorobenzoate (5.0 g, 94%) as a solid. LCMS ESI (+) m/z 249 (M+H). ¹HNMR (300 MHz, CDCl₃) δ 7.53 (dd, J=8.8, 1.8 Hz, 1H), 6.78 (dd, J=8.8, 6.3 Hz, 1H), 5.93 (s, 1H), 3.90 (s, 1H).

Step B: Preparation of methyl 2-amino-4-bromo-3-fluoro-5-iodobenzoate: To a mixture of iodine (7.16 g, 28 mmol) and silver sulfate (5.3 g, 17 mmol) in EtOH (200 mL), methyl 2-amino-4-bromo-3-fluorobenzoate (5.0 g, 20 mmol) was added and the resulting mixture was stirred at ambient temperature for 45 minutes. The solid was filtered off and washed with DCM, and the filtrate was concentrated under vacuum. The residue was dissolved in DCM and washed with 10% sodium thiosulphate solution and brine, and the resulting organic solution was dried over Na₂SO₄, filtered, and concentrated under vacuum to give methyl 2-amino-4-bromo-3-fluoro-5-iodobenzoate (6.66 g, 88% yield) as a yellow solid. LCMS ESI (+) m/z 373 (M+H). ¹HNMR (300 MHz, CDCl₃) δ 8.14 (d, J=1.9 Hz, 1H), 5.94 (s, 2H), 3.91 (s, 3H).

Step C: Preparation of methyl 2-acetamido-4-bromo-3-fluoro-5-iodobenzoate: Methyl 2-amino-4-bromo-3-fluoro-5-iodobenzoate (3.50 g, 9.4 mmol) and pyridine (2.3 ml, 28 mmol) were dissolved in DCM at 0° C. Acetyl chloride (0.79 mL, 11 mmol) was added and the reaction was warmed to ambient temperature and stirred at this temperature for 16 hours. The reaction mixture was concentrated under vacuum and the residue obtained was purified by silica gel column chromatography using ethyl acetate in hexanes (0% to 30%) as eluent to give methyl 2-acetamido-4-bromo-3-fluoro-5-iodobenzoate (2.7 g, 69%) as solid. LCMS ESI (+) m/z 417 (M+H). ¹HNMR (300 MHz, CDCl₃) δ 8.87 (s, 1H), 8.25 (s, 1H), 3.95 (s, 3H), 2.26 (s, 3H).

Step D: Preparation of methyl 2-acetamido-4-bromo-3-fluoro-5-(trifluoromethyl)benzoate: To a stirred solution of methyl 4-bromo-2-acetamido-3-fluoro-5-iodobenzoate (1.0 g, 2.4 mmol) and methyl fluorosulfonyldifluoroacetate (0.92 g, 0.72 mmol) in NMP (22.0 mL) at ambient temperature, CuI (0.14 g, 0.73 mmol) was added and the resulting mixture was stirred at 80° C. for 16 hours. After cooling to ambient temperature, the mixture was quenched with water and extracted with ethyl acetate. The organic layer was dried over Na₂SO₄, filtered, and concentrated and the crude material was purified by silica gel column chromatography using ethyl acetate in hexanes (0% to 20%) as eluent to give methyl 2-acetamido-4-bromo-3-fluoro-5-(trifluoromethyl)benzoate (0.64 g, 74%) as solid. LCMS ESI (+) m/z 358 (M+H). ¹HNMR (300 MHz, CDCl₃) δ 9.23 (s, 1H), 8.10 (s, TH), 3.93 (s, 3H), 2.28 (s, 3H).

Step E: Preparation of 2-acetamido-4-bromo-3-fluoro-5-(trifluoromethyl)benzoic acid: Methyl 4-bromo-2-acetamido-3-fluoro-5-(trifluoromethyl)benzoate (3.4 g, 9.49 mmol) was dissolved in THF (56 mL) and water (14 mL) at ambient temperature, then LiOH (0.91 g, 38 mmol) was added. The resulting mixture was stirred at 80° C. for 2 hours. The reaction was diluted with water, acidified with 2M HCl to adjust to pH ˜4 and then extracted with ethyl acetate (2×25 mL). The organic layer was washed with brine, dried over Na₂SO₄, filtered and concentrated in vacuum to afford 4-bromo-2-acetamido-3-fluoro-5-(trifluoromethyl)benzoic acid as solid (3.0 g, 92%). ¹HNMR (300 MHz, CD₃OD) δ 8.10 (s, 1H), 8.01 (s, 1H), 2.21 (s, 3H).

Step F: Preparation of 2-amino-4-bromo-3-fluoro-5-(trifluoromethyl)benzoic acid: 4-Bromo-2-acetamido-3-fluoro-5-(trifluoromethyl)benzoic acid (0.50 g, 1.45 mmol) was dissolved in a 3 M solution of HCl in MeOH (0.064 mL, 1.74 mmol) and refluxed at 80° C. for 2 hours. After cooling to ambient temperature, the reaction mixture was concentrated under vacuum to provide 2-amino-4-bromo-3-fluoro-5-(trifluoromethyl)benzoic acid as a solid (0.40 g, 91%). LCMS ESI (−) m/z 300 (M−H). ¹HNMR (300 MHz, CD₃OD) δ 7.86 (s, 1H).

Step G: Preparation of 7-bromo-8-fluoro-6-(trifluoromethyl)quinazolin-4-ol: To a solution of 2-amino-4-bromo-3-fluoro-5-(trifluoromethyl)benzoic acid (1.00 eq, 3.16 g, 10.5 mmol) in 2-ethoxyethanol (50 mL) was added formamidine acetate (2.00 eq, 2.18 g, 20.9 mmol) at 25° C. After addition, the reaction mixture was stirred at 140° C. for 16 hours. After cooling to ambient temperature, the mixture was evaporated in vacuo. Then 40 mL of water was added to the mixture, and the mixture was extracted with DCM (3×60 mL). The combined organic phase was washed with brine, dried over Na₂SO₄, and concentrated. The residue was purified with silica gel column chromatography (DCM-DCM/MeOH=10/1) to give 7-bromo-8-fluoro-6-(trifluoromethyl)quinazolin-4-ol (1.30 g, 3.89 mmol, 37.2% yield) as a solid. LCMS ESI (+) m/z 311 (M+H).

Step H: Preparation of 7-bromo-4-chloro-8-fluoro-6-(trifluoromethyl)quinazoline: To a solution of 7-bromo-8-fluoro-6-(trifluoromethyl)quinazolin-4-ol (1.00 eq, 400 mg, 1.29 mmol) in phosphoryl trichloride (41.6 eq, 5.0 mL, 53.5 nmol) was added DIEA (5.00 eq, 1.1 mL, 6.43 mmol) at 25° C. under N₂. The resulting mixture was stirred at 100° C. for 2 hours. After cooling to ambient temperature, the mixture was evaporated in vacuo to give the crude product (1.6 g) as an oil. LCMS ESI (+) m/z 329 (M+H).

Step I: Preparation of tert-butyl 3-[7-bromo-8-fluoro-6-(trifluoromethyl)quinazolin-4-yl]-3,8-diazabicyclo[3.2.1]octane-8-carboxylate: To a solution of 7-bromo-4-chloro-8-fluoro-6-(trifluoromethyl)quinazoline (1.00 eq, 1.60 g, 4.86 mmol) and TEA (3.00 eq, 1.47 g, 14.6 mmol) in DCM (20 mL) was added tert-butyl 3,8-diazabicyclo[3.2.1]octane-3-carboxylate (1.00 eq, 1.03 g, 4.86 mmol) at 0° C. under N₂. The resulting mixture was stirred at 25° C. for 16 hours. Then 40 mL of water was added to the mixture and extracted with EA (3×60 mL). The combined organic phase was washed with brine, dried over Na₂SO₄, and concentrated. The residue was purified by passing through a silica gel column (PE-PE/EA=5/1) to give tert-butyl 3-[7-bromo-8-fluoro-6-(trifluoromethyl)quinazolin-4-yl]-3,8-diazabicyclo[3.2.1]octane-8-carboxylate (260 mg, 0.494 mmol, 10.2% yield) as a solid. LCMS ESI (+) m/z 505 (M+H).

Step J: Preparation of tert-butyl 3-[7-[2-(tert-butoxycarbonylamino)-3-cyano-7-fluoro-benzothiophen-4-yl]-8-fluoro-6-(trifluoromethyl)quinazolin-4-yl]-3,8-diazabicyclo[3.2.1]octane-8-carboxylate: To a solution of tert-butyl 3-[7-bromo-8-fluoro-6-(trifluoromethyl)quinazolin-4-yl]-3,8-diazabicyclo[3.2.1]octane-8-carboxylate (1.00 eq, 310 mg, 0.613 mmol) in 1,4-dioxane (9 mL) was added tert-butyl N-[3-cyano-4-(5,5-dimethyl-1,3,2-dioxaborinan-2-yl)-7-fluoro-benzothiophen-2-yl]carbamate (1.50 eq, 372 mg, 0.92 mmol), Cs₂CO₃ (3.00 eq, 600 mg, 1.84 mmol) and Pd(DPEPhos)Cl₂ (0.20 eq, 88 mg, 0.12 mmol) under N₂. The resulting mixture was stirred at 95° C. for 16 hours under N₂. After cooling to ambient temperature, 20 mL of water was added the mixture and extracted with EA (3×20 mL). The combined organic phase was washed with brine, dried over Na₂SO₄ and concentrated. The residue was purified by passing through a silica gel column (PE-PE/EA=10/1) to give crude product. The crude product was purified by preparative-HPLC to give tert-butyl 3-[7-[2-(tert-butoxycarbonylamino)-3-cyano-7-fluoro-benzothiophen-4-yl]-8-fluoro-6-(trifluoromethyl)quinazolin-4-yl]-3,8-diazabicyclo[3.2.1]octane-8-carboxylate (30 mg, 0.0418 mmol, 6.8% yield) as a solid. LCMS ESI (+) m/z 717 (M+H).

Step K: Preparation of 2-amino-4-[4-(3,8-diazabicyclo[3.2.1]octan-3-yl)-8-fluoro-6-(trifluoromethyl)quinazolin-7-yl]-7-fluoro-benzothiophene-3-carbonitrile: To a solution of tert-butyl 3-[7-[2-(tert-butoxycarbonylamino)-3-cyano-7-fluoro-benzothiophen-4-yl]-8-fluoro-6-(trifluoromethyl)quinazolin-4-yl]-3,8-diazabicyclo[3.2.1]octane-8-carboxylate (1.00 eq, 20 mg, 0.0279 mmol) in DCM (3 mL) was added TFA (465 eq, 1.0 mL, 13.0 mmol) at 25° C. The resulting mixture was stirred at 25° C. for 4 hours. The resulting mixture was concentrated under vacuo and the residue was purified by preparative-HPLC to give 2-amino-4-[4-(3,8-diazabicyclo[3.2.1]octan-3-yl)-8-fluoro-6-(trifluoromethyl)quinazolin-7-yl]-7-fluoro-benzothiophene-3-carbonitrile (6.1 mg, 0.0116 mmol, 41.48% yield) as a solid. LCMS ESI (+) m/z 517 (M+H). ¹H NMR (400 MHz, CD₃OD) δ 8.74 (s, 1H), 8.19 (s, 1H), 7.17-7.33 (m, 1H), 7.03 (t, 1H), 4.81 (d, 1H), 4.63 (d, 1H), 4.09 (d, 2H), 3.94 (d, 1H), 3.84 (d, 1H), 1.85-2.14 (m, 4H).

### Synthetic Example 2: Synthesis of 4-(4-((1R,5S)-3,8-diazabicyclo[3.2.1]octan-3-yl)-2-(dimethylamino)-8-fluoro-6-(trifluoromethyl)quinazolin-7-yl)-2-amino-7-fluorobenzo[b]thiophene-3-carbonitrile (Compound 10)

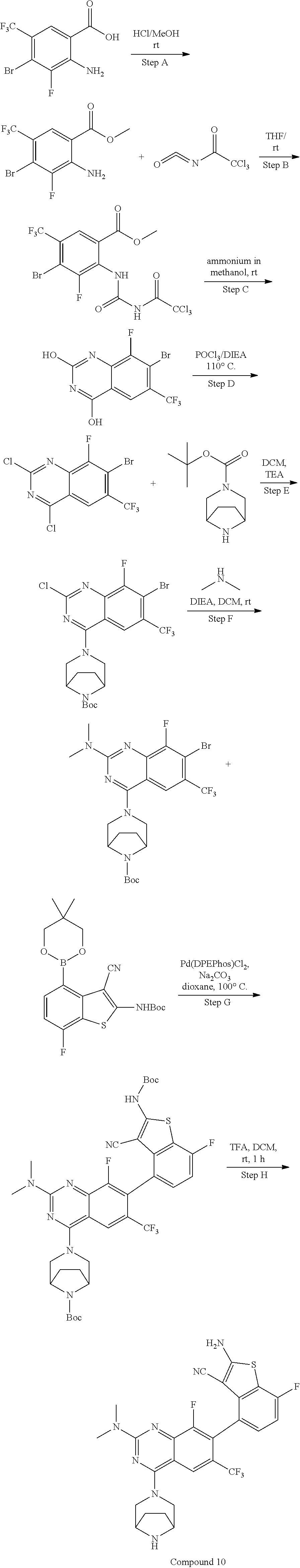

Step A: Preparation of 2-amino-4-bromo-3-fluoro-5-(trifluoromethyl)benzoate: A mixture of methyl 2-acetamido-4-bromo-3-fluoro-5-(trifluoromethyl)benzoate (1.20 g, 3.35 mmol) in 3 M HCl in MeOH was heated at 60° C. for 2 hours. After cooling to ambient temperature, the mixture was concentrated, and the crude product was partitioned between EtOAc and saturated NaHCO₃. The organic layer was separated, dried over sodium sulfate, filtered, and concentrated to provide 2-amino-4-bromo-3-fluoro-5-(trifluoromethyl)benzoate (1.00 g, 94%) as an oil. LCMS ESI (+) m/z 316.9 (M+H). ¹H NMR (300 MHz, CDCl₃) δ 8.01 (s, 1H), 6.28 (s, 2H), 3.94 (s, 3H).

Step B: Preparation of methyl 4-bromo-3-fluoro-2-(3-(2,2,2-trichloroacetyl)ureido)-5-(trifluoromethyl)benzoate: To a mixture of methyl 2-amino-4-bromo-3-fluoro-5-(trifluoromethyl)benzoate (0.80 g, 2.53 mmol) in THF (4.2 mL) was added trichloroethanecarbonyl isocyanate (0.45 mL, 3.79 mmol) at ambient temperature. After 15 minutes, the reaction mixture was concentrated, followed by the addition of MTBE, and the solid formed was collected and washed with MTBE to provide methyl 4-bromo-3-fluoro-2-(3-(2,2,2-trichloroacetyl)ureido)-5-(trifluoromethyl)benzoate (0.71 g, 56%). LCMS ESI (+) m/z 529.99 (M+Na). ¹H NMR (300 MHz, CDCl₃) δ 10.91 (s, 1H), 8.65 (s, 1H), 8.23 (s, 1H), 4.03 (s, 3H).

Step C: Preparation of 7-bromo-8-fluoro-6-(trifluoromethyl)quinazoline-2,4-diol: To a solution of methyl 4-bromo-3-fluoro-2-(3-(2,2,2-trichloroacetyl)ureido)-5-(trifluoromethyl)benzoate (0.71 g, 1.40 mmol) in methanol (7.0 mL) was added 7 M solution of ammonia in methanol (0.46 mL, 3.23 mmol) at ambient temperature and stirred at ambient temperature for 1 hour. The mixture was concentrated under reduced pressure to provide a solid which was co-evaporated with ether to provide 7-bromo-8-fluoro-6-(trifluoromethyl)quinazoline-2,4-diol (0.60 g, 100%) as a solid. LCMS ESI (+) m/z 260.0 (M+H). ¹H NMR (300 MHz, DMSO-d₆) δ 8.41 (s, 1H), 8.28 (s, 1H), 7.99 (s, 1H).

Step D: Preparation of 7-bromo-2,4-dichloro-8-fluoro-6-(trifluoromethyl)quinazoline: To a stirring solution of phosphorus oxychloride (0.97 mL, 10.5 mmol) and Hunig's base (0.40 mL, 2.29 mmol) was added 7-bromo-8-fluoro-6-(trifluoromethyl)quinazoline-2,4-diol (0.15 g, 0.46 mmol) at 0° C. After addition, the resulting mixture was stirred at 110° C. for 1 hour. After cooling down to ambient temperature, the mixture was evaporated and co-evaporated with chloroform to give 7-bromo-2,4-dichloro-8-fluoro-6-(trifluoromethyl)quinazoline which used as such for the next step. ¹H NMR (300 MHz, CDCl₃) δ 8.45 (s, 1H).

Step E: Preparation of tert-butyl-3-(7-bromo-2,6-dichloro-8-fluoro-quinazolin-4-yl)-3,8-diazabicyclo[3.2.1]octane-8-carboxylate: To a mixture of 7-bromo-2,4,6-trichloro-8-fluoro-quinazoline (500 mg, 1.51 mmol) in DCM (10 mL) was added tert-butyl 3,8-diazabicyclo[3.2.1]octane-8-carboxylate (386 mg, 1.82 mmol) and Et₃N (459 mg, 4.54 mmol) at ambient temperature, and the mixture was stirred at ambient temperature for 2 hours. Ethyl acetate (50 mL) was added and the mixture was washed with saturated brine solution. The organic layers were then separated, dried (MgSO₄), filtered and concentrated under reduced pressure. The crude was then purified by flash chromatography on silica gel to give tert-butyl-3-(7-bromo-2,6-dichloro-8-fluoro-quinazolin-4-yl)-3,8-diazabicyclo[3.2.1]octane-8-carboxylate (608 mg, 79%). LCMS (ES+) m/z 505 (M+H).

Step F: Preparation of tert-butyl (1R,5S)-3-(7-bromo-2-(dimethylamino)-8-fluoro-6-(trifluoromethyl)quinazolin-4-yl)-3,8-diazabicyclo[3.2.1]octane-8-carboxylate: To a solution of dimethylamine (3.00 eq, 50.11 mg, 1.11 mmol) in DCM (5 mL) was added DIEA (6.0 eq, 287.3 mg, 2.22 mmol) and tert-butyl 3-[7-bromo-2-chloro-8-fluoro-6-(trifluoromethyl)quinazolin-4-yl]-3,8-diazabicyclo[3.2.1]octane-8-carboxylate (1.0 eq, 200 mg, 0.37 mmol). The resulting mixture was stirred at 25° C. for 12 hours. The mixture was quenched with water (50 mL) and extracted with DCM (10 mL×3). The organic layer was dried over Na₂SO₄ and concentrated to give a residue. The residue was purified by preparative TLC (PE/EA=5:1) to give tert-butyl (1R,5S)-3-(7-bromo-2-(dimethylamino)-8-fluoro-6-(trifluoromethyl)quinazolin-4-yl)-3,8-diazabicyclo[3.2.1]octane-8-carboxylate (188.00 mg, 0.33 mmol, 87.9% yield) as a solid. LCMS ESI (+) m/z 548.1 (M+H).

Step G: Preparation of tert-butyl (1R,5S)-3-(7-(2-((tert-butoxycarbonyl)amino)-3-cyano-7-fluorobenzo[b]thiophen-4-yl)-2-(dimethylamino)-8-fluoro-6-(trifluoromethyl)quinazolin-4-yl)-3,8-diazabicyclo[3.2.1]octane-8-carboxylate: To a solution of tert-butyl N-[3-cyano-4-(5,5-dimethyl-1,3,2-dioxaborinan-2-yl)-7-fluoro-benzothiophen-2-yl]carbamate (1.5 eq, 55.0 mg, 0.14 mmol) in 1,4-dioxane (5 mL) was added tert-butyl 3-[7-bromo-2-(dimethylamino)-8-fluoro-6-(trifluoromethyl)quinazolin-4-yl]-3,8-diazabicyclo[3.2.1]octane-8-carboxylate (1.0 eq, 50.0 mg, 0.091 mmol), Na₂CO₃ (3.0 eq, 29.0 mg, 0.27 mmol) and Pd(DPEphos)Cl₂ (0.2 eq, 13.06 mg, 0.018 mmol) under N₂. The mixture was stirred at 100° C. for 12 hours under N₂. The mixture was quenched with water (10 mL) and extracted with DCM (10 mL×3). The organic layer was dried over Na₂SO₄ and concentrated to give a residue. The residue was purified by preparative TLC (DCM/MeOH=15:1) to give tert-butyl (1R,5S)-3-(7-(2-((tert-butoxycarbonyl)amino)-3-cyano-7-fluorobenzo[b]thiophen-4-yl)-2-(dimethylamino)-8-fluoro-6-(trifluoromethyl)quinazolin-4-yl)-3,8-diazabicyclo[3.2.1]octane-8-carboxylate (15.0 mg, 0.018 mmol, 19.5% yield) as a yellow solid. LCMS ESI (+) m/z 760.2 (M+H).

Step H: Preparation of 4-(4-((1R,5S)-3,8-diazabicyclo[3.2.1]octan-3-yl)-2-(dimethylamino)-8-fluoro-6-(trifluoromethyl)quinazolin-7-yl)-2-amino-7-fluorobenzo[b]thiophene-3-carbonitrile: To a solution of tert-butyl 3-[7-[2-(tert-butoxycarbonylamino)-3-cyano-7-fluoro-benzothiophen-4-yl]-2-(dimethylamino)-8-fluoro-6-(trifluoromethyl)quinazolin-4-yl]-3,8-diazabicyclo[3.2.1]octane-8-carboxylate (1.0 eq, 15.0 mg, 0.02 mmol) in DCM (5 mL) was added TFA (5 mL). The mixture was stirred for 1 hour at 25° C. The mixture was concentrated in vacuo to give a residue. The residue was purified by preparative-HPLC to give 4-(4-((1R,5S)-3,8-diazabicyclo[3.2.1]octan-3-yl)-2-(dimethylamino)-8-fluoro-6-(trifluoromethyl)quinazolin-7-yl)-2-amino-7-fluorobenzo[b]thiophene-3-carbonitrile (2 mg, 0.0035 mmol, 17.9% yield) as a solid. LCMS ESI (+) m/z 561.3 (M+H). ¹H NMR (400 MHz, CD₃OD) δ 8.00 (s, 1H), 7.23-7.20 (m, 1H), 7.05-7.00 (m, 1H), 4.71-4.67 (d, 1H), 4.56-4.52 (d, 1H), 4.25-4.23 (m, 2H), 3.93-3.89 (d, 1H), 3.83-3.80 (d, 1H), 3.33 (s, 3H), 3.31 (s, 3H), 2.19-2.13 (m, 4H).

### Synthetic Example 3: Synthesis of N-(7-(2-amino-3-cyano-7-fluorobenzo[b]thiophen-4-yl)-4-((1R,5S)-3,8-diazabicyclo[3.2.1]octan-3-yl)-8-fluoro-6-(trifluoromethyl)quinazolin-2-yl)-N-methylacetamide (Compound 21)

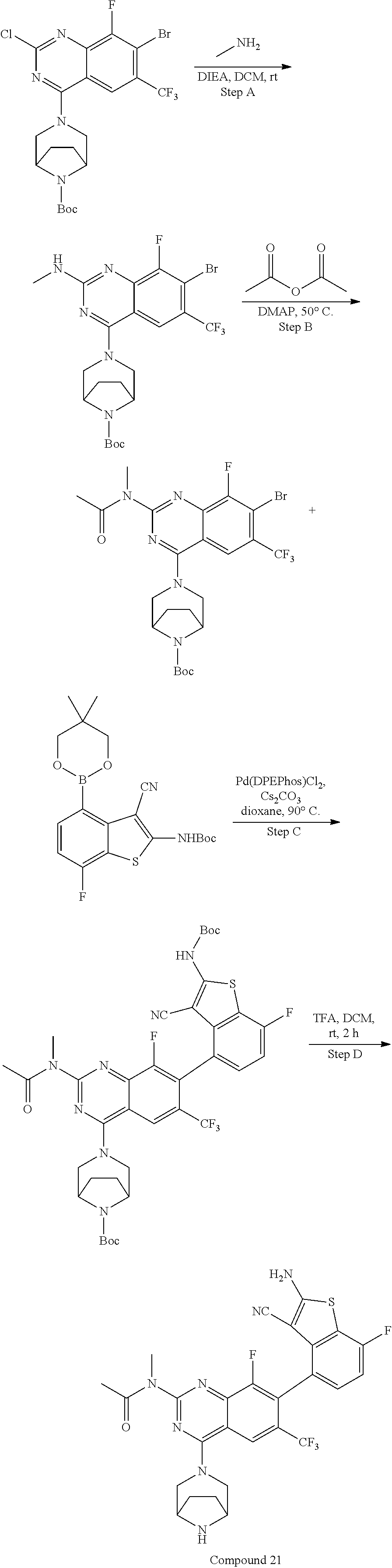

Step A: Preparation of tert-butyl 3-[7-bromo-8-fluoro-2-(methylamiino)-6-(trifluoromethyl)quinazolin-4-yl]-3,8-diazabicyclo[3.2.1]octane-8-carboxylate: To a solution of tert-butyl 3-[7-bromo-2-chloro-8-fluoro-6-(trifluoromethyl)quinazolin-4-yl]-3,8-diazabicyclo[3.2.1]octane-8-carboxylate (1.0 eq, 500 mg, 0.93 mmol) in DCM (10 mL) was added methylamine in EtOH (30%-40%) (3.0 eq, 57 mg, 2.78 mmol) and DIEA (5.0 eq, 0.82 mL, 4.63 mmol). After addition, the reaction mixture was stirred at 20° C. for 16 hours. The reaction mixture was quenched with water, extracted with DCM (3×20 mL). The combined organic phase was washed with brine, dried over Na₂SO₄ and concentrated. The residue was purified by preparative-TLC (EA/PE=1:4) to give tert-butyl 3-[7-bromo-8-fluoro-2-(methylamino)-6-(trifluoromethyl)quinazolin-4-yl]-3,8-diazabicyclo[3.2.1]octane-8-carboxylate (451 mg, 0.782 mmol, 84.4% yield) as a solid. LCMS ESI (+) m/z 534.1 (M+H).

Step B: Preparation of tert-butyl 3-[2-[acetyl(methyl)amino]-7-bromo-8-fluoro-6-(trifluoromethyl)quinazolin-4-yl]-3,8-diazabicyclo[3.2.1]octane-8-carboxylate: To a solution of tert-butyl 3-[7-bromo-8-fluoro-2-(methylamino)-6-(trifluoromethyl)quinazolin-4-yl]-3,8-diazabicyclo[3.2.1]octane-8-carboxylate (1.0 eq, 100 mg, 0.19 mmol) in acetic anhydride (283 eq, 5.0 mL, 53.0 mmol) was added DMAP (0.20 eq, 4.6 mg, 0.037 mmol). After addition, the reaction mixture was stirred at 50° C. for 3 hours. The reaction mixture was quenched with water, extracted with EA (3×30 mL). The combined organic phase was washed with brine, dried over Na₂SO₄ and concentrated. The residue was purified by preparative-TLC (EA/PE=1:4) to give crude tert-butyl 3-[2-[acetyl(methyl)amino]-7-bromo-8-fluoro-6-(trifluoromethyl)quinazolin-4-yl]-3,8-diazabicyclo[3.2.1]octane-8-carboxylate (125 mg, 0.203 mmol, 100% yield). LCMS ESI (+) m/z 576.12 (M+H).

Step C: Preparation of tert-butyl 3-[2-[acetyl(methyl)amino]-7-[2-(tert-butoxycarbonylamino)-3-cyano-7-fluoro-benzothiophen-4-yl]-8-fluoro-6-(trifluoromethyl)quinazolin-4-yl]-3,8-diazabicyclo[3.2.1]octane-8-carboxylate: To a solution of tert-butyl 3-[2-[acetyl(methyl)amino]-7-bromo-8-fluoro-6-(trifluoromethyl)quinazolin-4-yl]-3,8-diazabicyclo[3.2.1]octane-8-carboxylate (1.0 eq, 100 mg, 0.17 mmol) and tert-butyl N-[3-cyano-4-(5,5-dimethyl-1,3,2-dioxaborinan-2-yl)-7-fluoro-benzothiophen-2-yl]carbamate (1.5 eq, 105 mg, 0.26 mmol) in 1,4-dioxane (5 mL) was added Pd(DPEPhos)Cl₂ (0.40 eq, 30 mg, 0.070 mmol) and Cs₂CO₃ (3.0 eq, 169.5 mg, 0.52 mmol). After addition, the reaction mixture was stirred at 90° C. for 3 hours under N₂. After cooling to ambient temperature, the reaction mixture was quenched with water and extracted with EA (3×30 mL). The combined organic phase was washed with brine, dried over Na₂SO₄, and concentrated. The residue was purified by preparative-TLC (EA/PE=1:2) to give tert-butyl 3-[2-[acetyl(methyl)amino]-7-[2-(tert-butoxycarbonylamino)-3-cyano-7-fluoro-benzothiophen-4-yl]-8-fluoro-6-(trifluoromethyl)quinazolin-4-yl]-3,8-diazabicyclo[3.2.1]octane-8-carboxylate (35 mg, 0.0386 mmol, 22.2% yield) as a yellow solid. LCMS ESI (+) m/z 788.26 (M+H).

Step D: Preparation of N-[7-(2-amino-3-cyano-7-fluoro-benzothiophen-4-yl)-4-(3,8-diazabicyclo[3.2.1]octan-3-yl)-8-fluoro-6-(trifluoromethyl)quinazolin-2-yl]-N-methyl-acetamide: To a solution of tert-butyl 3-[2-[acetyl(methyl)amino]-7-[2-(tert-butoxycarbonylamino)-3-cyano-7-fluoro-benzothiophen-4-yl]-8-fluoro-6-(trifluoromethyl)quinazolin-4-yl]-3,8-diazabicyclo[3.2.1]octane-8-carboxylate (1.0 eq, 30 mg, 0.038 mmol) in DCM (6 mL) was added TFA (2.0 mL, 26.8 mmol) at 0° C. under N₂. After addition, the reaction mixture was stirred at 20° C. for 2 hours. The reaction mixture was quenched with NaHCO₃ and extracted with DCM (3×30 mL). The combined organic phase was washed with brine, dried over Na₂SO₄, concentrated. The residue was purified by preparative-HPLC to give N-[7-(2-amino-3-cyano-7-fluoro-benzothiophen-4-yl)-4-(3,8-diazabicyclo[3.2.1]octan-3-yl)-8-fluoro-6-(trifluoromethyl)quinazolin-2-yl]-N-methyl-acetamide (9.5 mg, 0.0154 mmol, 40.4% yield) as a solid. LCMS ESI (+) m/z 588.15 (M+H). ¹H NMR (400 MHz, CD₃OD) δ 8.16 (s, 1H), 7.25-7.21 (m, 1H), 7.02 (t, J=8.8 Hz, 1H), 4.65-4.54 (m, 2H), 4.01 (s, 2H), 3.78 (t, J=14.4 Hz, 2H), 3.51 (s, 3H), 2.50 (s, 3H), 2.05-1.95 (m, 4H).

### Synthetic Example 4: Synthesis of 4-(4-((1R,5S)-3,8-diazabicyclo[3.2.1]octan-3-yl)-8-fluoro-2-methyl-6-(trifluoromethyl)quinazolin-7-yl)-2-amino-7-fluorobenzo[b]thiophene-3-carbonitrile (Compound 22)

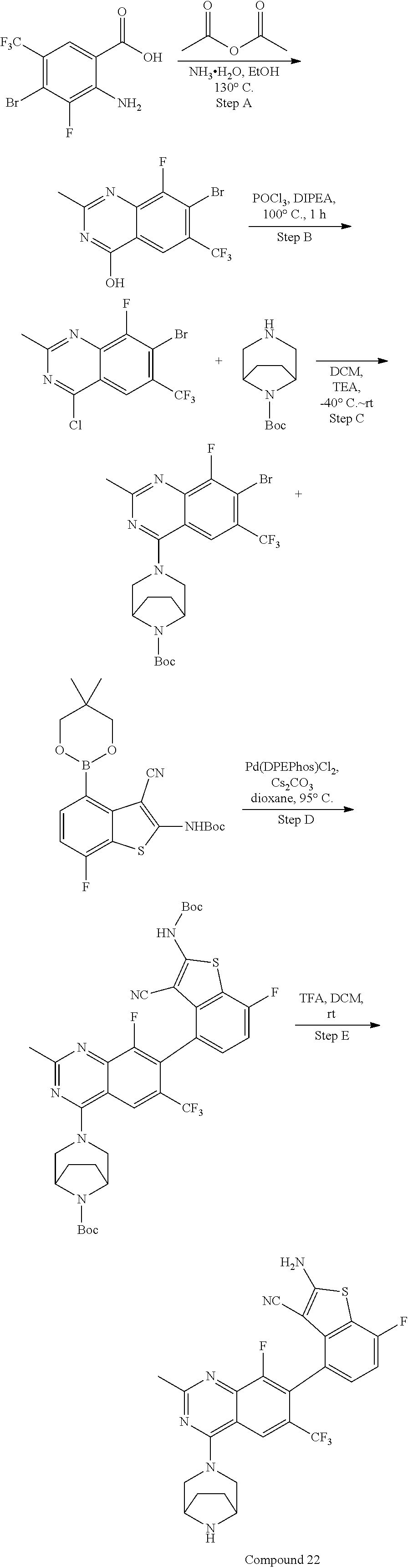

Step A: Preparation of 7-bromo-8-fluoro-2-methyl-6-(trifluoromethyl)quinazolin-4-ol: A solution of 2-amino-4-bromo-3-fluoro-5-(trifluoromethyl) benzoic acid (1.0 eq, 900 mg, 2.98 mmol) in Ac₂O (32.0 eq, 9.0 mL, 95.4 mmol) was stirred at 130° C. for 16 hours. After cooling to ambient temperature, the reaction was concentrated and was directly used in the next step without further purification. A solution of the above crude product and ammonium hydroxide (10 mL) in ethanol (10 mL) was stirred at 80° C. for 16 hours. After cooling to ambient temperature, the mixture was diluted with water and extracted with EA (4×10 mL). The combined organic phase was washed with brine, dried over sodium sulfate, filtered and concentrated. The residue was purified by silica gel column chromatography eluting with EA in petroleum from 0% to 50% to afford 7-bromo-8-fluoro-2-methyl-6-(trifluoromethyl)quinazolin-4-ol (440 mg, 1.26 mmol, 42.2% yield) as a solid. LCMS ESI (+) m/z 325.1 (M+H).

Step B: Preparation of 7-bromo-4-chloro-8-fluoro-2-methyl-6-(trifluoromethyl)quinazoline: A solution of 7-bromo-8-fluoro-2-methyl-6-(trifluoromethyl)quinazolin-4-ol (1.0 eq, 440 mg, 1.26 mmol), DIEA (2.0 eq, 0.44 mL, 2.52 mmol) and phosphorus oxychloride (2.0 eq, 0.24 mL, 2.52 mmol) in toluene (10 mL) was stirred at 100° C. for 1 hour. After cooling to ambient temperature, the reaction was concentrated to dryness, washed three times with methyl tert-butyl ether and the supernatant was concentrated to obtain 7-bromo-4-chloro-8-fluoro-2-methyl-6-(trifluoromethyl)quinazoline (400 mg, 0.967 mmol, 71.4% yield), which was used in the next step without further purification. LCMS ESI (+) m/z 343.0 (M+H).

Step C: Preparation of tert-butyl (1R,5S)-3-(7-bromo-8-fluoro-2-methyl-6-(trifluoromethyl)quinazolin-4-yl)-3,8-diazabicyclo[3.2.1]octane-8-carboxylate: To a solution of 7-bromo-4-chloro-8-fluoro-2-methyl-6-(trifluoromethyl)quinazoline (1.0 eq, 400 mg, 0.967 mmol) in DCM (6 mL) was added TEA (3.00 eq, 294 mg, 2.91 mmol) until pH>7, then tert-butyl 3,8-diazabicyclo[3.2.1]octane-8-carboxylate (1.0 eq. 205 mg, 0.97 mmol) in DCM was added at −40° C. under N₂. After addition, the reaction mixture was stirred at room temperature for 16 hours. The resulting mixture was diluted with water and extracted with DCM (4×10 mL). The combined organic phase was washed with brine, dried over sodium sulfate, filtered and concentrated. The residue was purified by preparative-TLC to give tert-butyl 3-[7-bromo-8-fluoro-2-methyl-6-(trifluoromethyl)quinazolin-4-yl]-3,8-diazabicyclo[3.2.1]octane-8-carboxylate (340 mg, 0.622 mmol, 71.2% yield) as a solid. LCMS ESI (+) m/z 519.2 (M+H).

Step D: Preparation of tert-butyl (1R,5S)-3-(7-(2-((tert-butoxycarbonyl)amino)-3-cyano-7-fluorobenzo[b]thiophen-4-yl)-8-fluoro-2-methyl-6-(trifluoromethyl)quinazolin-4-yl)-3,8-diazabicyclo[3.2.1]octane-8-carboxylate: A solution of tert-butyl 3-[7-bromo-8-fluoro-2-methyl-6-(trifluoromethyl)quinazolin-4-yl]-3,8-diazabicyclo[3.2.1]octane-8-carboxylate (1.0 eq, 200 mg, 0.39 mmol), tert-butyl N-[3-cyano-4-(5,5-dimethyl-1,3,2-dioxaborinan-2-yl)-7-fluoro-benzothiophen-2-yl]carbamate (1.50 eq, 234 mg, 0.58 mmol), cesium carbonate (3.0 eq, 378 mg, 1.16 mmol), and Pd(DPEPhos)Cl₂ (0.40 eq, 110 mg, 0.15 mmol) in 1,4-dioxane (1 mL) was stirred at 95° C. for 16 hours under argon. After cooling to ambient temperature, the mixture was diluted with water and extracted with EA (3×10 mL). The combined extracts were washed with brine, dried over sodium sulfate, filtered, and concentrated. The residue was purified by preparative-TLC to give tert-butyl 3-[7-[2-(tert-butoxycarbonylamino)-3-cyano-7-fluoro-benzothiophen-4-yl]-8-fluoro-2-methyl-6-(trifluoromethyl) quinazolin-4-yl]-3,8-diazabicyclo[3.2.1]octane-8-carboxylate (105 mg, 0.138 mmol, 35.8% yield) as a solid. LCMS ESI (+) m/z 731.1 (M+H).

Step E: Preparation of 4-(4-((1R,5S)-3,8-diazabicyclo[3.2.1]octan-3-yl)-8-fluoro-2-methyl-6-(trifluoromethyl)quinazolin-7-yl)-2-amino-7-fluorobenzo[b]thiophene-3-carbonitrile: A solution of tert-butyl 3-[7-[2-(tert-butoxycarbonylamino)-3-cyano-7-fluoro-benzothiophen-4-yl]-8-fluoro-2-methyl-6-(trifluoromethyl)quinazolin-4-yl]-3,8-diazabicyclo[3.2.1]octane-8-carboxylate (1.0 eq, 105 mg, 0.14 mmol) and TFA (142 eq, 1.5 mL, 19.5 mmol) in DCM (4 mL) was stirred at 25° C. for 4 hours. The reaction was concentrated in vacuo. The residue was purified by preparative-HPLC to give 2-amino-4-[4-(3,8-diazabicyclo[3.2.1]octan-3-yl)-8-fluoro-2-methyl-6-(trifluoromethyl)quinazolin-7-yl]-7-fluoro-benzothiophene-3-carbonitrile (20 mg, 0.0378 mmol, 27.6% yield) as a solid. LCMS ESI (+) m/z 531.0 (M+H). ¹H NMR (400 MHz, CD₃OD) δ 8.46 (s, 0.7H), 8.14 (s, 1H), 7.21-7.25 (m, 1H), 7.00-7.05 (m, 1H), 4.76 (d, 1H), 4.60 (d, 1H), 4.07 (d, 2H), 3.76-3.88 (m, 2H), 2.66 (s, 3H), 1.98-2.03 (m, 4H).

### Synthetic Example 5: Synthesis of 4-(4-((1R,5S)-3,8-diazabicyclo[3.2.1]octan-3-yl)-8-fluoro-2-((tetrahydro-2H-pyran-4-yl)methoxy)-6-(trifluoromethyl)quinazolin-7-yl)-2-amino-7-fluorobenzo[b]thiophene-3-carbonitrile (Compound 37)

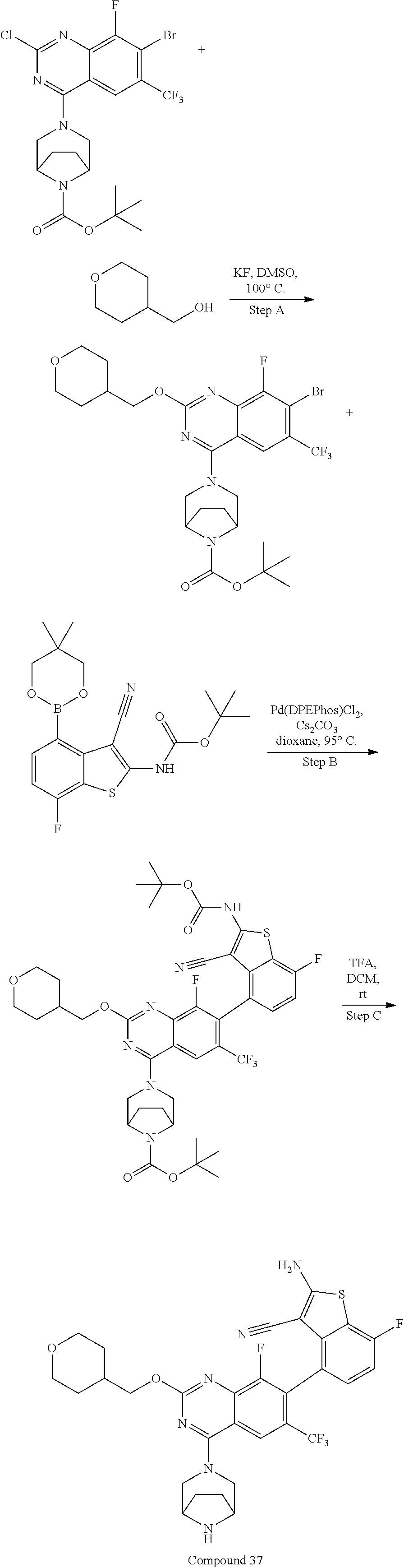

Step A: Preparation of tert-butyl (1R,5S)-3-(7-bromo-8-fluoro-2-((tetrahydro-2H-pyran-4-yl)methoxy)-6-(trifluoromethyl)quinazolin-4-yl)-3,8-diazabicyclo[3.2.1]octane-8-carboxylate: A solution of tert-butyl 3-[7-bromo-2-chloro-8-fluoro-6-(trifluoromethyl)quinazolin-4-yl]-3,8-diazabicyclo[3.2.1]octane-8-carboxylate (1.0 eq, 300 mg, 0.56 mmol), tetrahydropyran-4-ylmethanol (2.0 eq, 129 mg, 1.11 mmol), and KF (8.0 eq, 258 mg, 4.45 mmol) in DMSO (5 mL) was stirred at 100° C. for 16 hours under N₂. After cooling to ambient temperature, the mixture was diluted with water and extracted with EA (4×10 mL). The combined extracts were washed with brine, dried over sodium sulfate, filtered, and concentrated. The residue was purified by preparative-TLC to give tert-butyl 3-[7-bromo-8-fluoro-2-(tetrahydropyran-4-ylmethoxy)-6-(trifluoromethyl)quinazolin-4-yl]-3,8-diazabicyclo[3.2.1]octane-8-carboxylate (200 mg, 0.310 mmol, 55.8% yield) as a solid. LCMS ESI (+) m/z 621.0 (M+H).

Step B: Preparation of tert-butyl (1R,5S)-3-(7-(2-((tert-butoxycarbonyl)amino)-3-cyano-7-fluorobenzo[b]thiophen-4-yl)-8-fluoro-2-((tetrahydro-2H-pyran-4-yl)methoxy)-6-(trifluoromethyl)quinazolin-4-yl)-3,8-diazabicyclo[3.2.1]octane-8-carboxylate: A mixture of tert-butyl N-[3-cyano-4-(5,5-dimethyl-1,3,2-dioxaborinan-2-yl)-7-fluoro-benzothiophen-2-yl]carbamate (1.5 eq, 196 mg, 0.46 mmol), tert-butyl 3-[7-bromo-8-fluoro-2-(tetrahydropyran-4-ylmethoxy)-6-(trifluoromethyl)quinazolin-4-yl]-3,8-diazabicyclo[3.2.1]octane-8-carboxylate (1.0 eq, 200 mg, 0.31 mmol), K₂CO₃ (3.0 eq, 128.53 mg, 0.93 mmol), and Pd(DPEPhos)Cl₂ (0.40 eq, 88.77 mg, 0.12 mmol) in 1,4-dioxane (10 mL) was stirred at 95° C. for 16 hours under argon. After cooling to ambient temperature, the mixture was diluted with water and extracted with EA (4×10 mL). The combined extracts were washed with brine, dried over sodium sulfate, filtered, and concentrated. The residue was purified by preparative-TLC to give tert-butyl 3-[7-[2-(tert-butoxycarbonylamino)-3-cyano-7-fluoro-benzothiophen-4-yl]-8-fluoro-2-(tetrahydropyran-4-ylmethoxy)-6-(trifluoromethyl)quinazolin-4-yl]-3,8-diazabicyclo[3.2.1]octane-8-carboxylate (130 mg, 0.106 mmol, 41.2% yield) as a white solid. LCMS ESI (+) m/z 831.3 (M+H).

Step C: Preparation of 4-(4-((1R,5S)-3,8-diazabicyclo[3.2.1]octan-3-yl)-8-fluoro-2-((tetrahydro-2H-pyran-4-yl)methoxy)-6-(trifluoromethyl)quinazolin-7-yl)-2-amino-7-fluorobenzo[b]thiophene-3-carbonitrile: A solution of tert-butyl 3-[7-[2-(tert-butoxycarbonylamino)-3-cyano-7-fluoro-benzothiophen-4-yl]-8-fluoro-2-(tetrahydropyran-4-ylmethoxy)-6-(trifluoromethyl)quinazolin-4-yl]-3,8-diazabicyclo[3.2.1]octane-8-carboxylate (1.0 eq, 130 mg. 0.11 mmol) and TFA (86.3 eq, 0.68 mL, 8.83 mmol) in DCM (3 mL) was stirred at 25° C. for 4 hours. The reaction solution was adjusted to pH 8 with saturation sodium bicarbonate aqueous solution and extracted with EA (4×10 mL). The combined organic phase was washed with brine, dried over sodium sulfate, and concentrated. The residue was purified by preparative-TLC to give 2-amino-4-[4-(3,8-diazabicyclo[3.2.1]octan-3-yl)-8-fluoro-2-(tetrahydropyran-4-ylmethoxy)-6-(trifluoromethyl)quinazolin-7-yl]-7-fluoro-benzothiophene-3-carbonitrile (31 mg, 0.0469 mmol, 45.8% yield) as a solid. LCMS ESI (+) m/z 631.2 (M+H). ¹H NMR (400 MHz, CD₃OD) δ 8.10 (s, 1H), 7.19-7.22 (m, 1H), 7.00 (t, 1H), 4.56 (t, 1H), 4.44 (d, 1H), 4.31 (d, 2H), 3.96-4.00 (m, 2H), 3.62-3.72 (m, 4H), 3.46 (t, 2H), 2.10-2.16 (m, 1H), 1.77-1.80 (m, 6H), 1.41-1.52 (m, 2H).

Table 2 includes selected compounds of the present disclosure.

TABLE 2
Selected compounds of the present disclosure.

| Compound | | |
|---|---|---|
| No. | Structure | Analytical data |
| 1 | 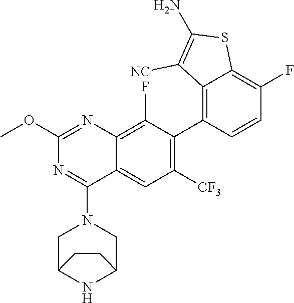 [SMILES: COc1nc(N2CC3CCC(C2)N3)c2cc(C(F)(F)F)c(-c3ccc(F)c4sc(N)c(C#N)c34)c(F)c2n1] | LCMS m/z [M + 1]: 547.0 ¹HNMR (400 MHz, CD₃OD) δ 8.15 (s, 1H), 7.23 (t, 1H), 7.04 (t, 1H), 4.82 (d, 1H), 4.68 (d, 1H), 4.25 (s, 2H), 4.12 (s, 3H), 3.95 (q, 2H), 2.14 (s, 4H). |
| 2 | 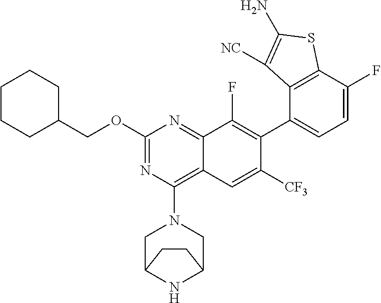 [SMILES: N#Cc1c(N)sc2c(F)ccc(-c3c(C(F)(F)F)cc4c(N5CC6CCC(C5)N6)nc(OCC5CCCCC5)nc4c3F)c12] | LCMS m/z [M + 1]: 629.0. ¹HNMR (400 MHz, CD₃OD) δ 8.11 (s, 1H), 7.23-7.20 (m, 1H), 7.02 (t, 1H), 4.75 (d, 1H), 4.62 (d, 1H), 4.29 (d, 2H), 4.23 (s, 2H), 3.95- 3.84 (m, 2H), 2.12 (s, 4H), 1.90-1.70 (m, 6H), 1.38-1.22 (m, 3H), 1.17-1.08 (m, 2H). |
| 3 | 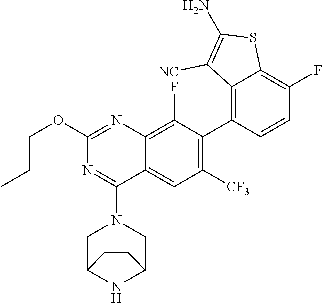 [SMILES: CCCOc1nc(N2CC3CCC(C2)N3)c2cc(C(F)(F)F)c(-c3ccc(F)c4sc(N)c(C#N)c34)c(F)c2n1] | LCMS m/z [M + 1]: 575.0 ¹HNMR (400 MHz, CD₃OD) δ 8.13 (s, 1H), 7.21-7.26 (m, 1H), 7.04 (t, 1H), 4.76 (d, 1H), 4.63 (d, 1H), 4.46 (t, 2H), 4.21-4.29 (m, 2H), 3.82-3.97 (m, 2H), 2.07-2.22 (m, 4H), 1.82-1.91 (m, 2H), 1.09 (t, 3H). |
| 4 | 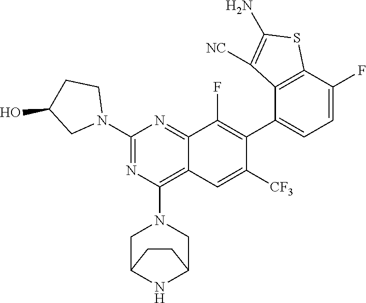 [SMILES: N#Cc1c(N)sc2c(F)ccc(-c3c(C(F)(F)F)cc4c(N5CC6CCC(C5)N6)nc(N5CC[C@H](O)C5)nc4c3F)c12] | LCMS m/z [M + 1]: 602.0 ¹HNMR (400 MHz, CD₃OD) δ 8.04 (s, 1H), 7.22 (t, 1H), 7.03 (t, 1H), 4.72-4.57 (m, 3H), 4.25 (s, 2H), 4.15-3.83 (m, 6H), 2.25-2.02 (m, 6H). |
| 4a | 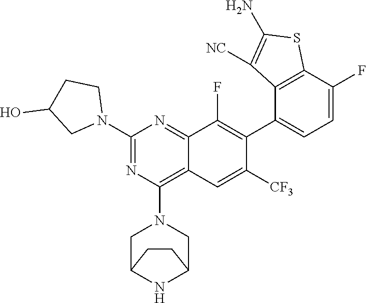 [SMILES: N#Cc1c(N)sc2c(F)ccc(-c3c(C(F)(F)F)cc4c(N5CC6CCC(C5)N6)nc(N5CCC(O)C5)nc4c3F)c12] | |
| 5 | 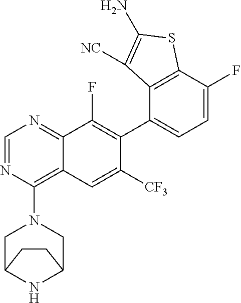 [SMILES: N#Cc1c(N)sc2c(F)ccc(-c3c(C(F)(F)F)cc4c(N5CC6CCC(C5)N6)ncnc4c3F)c12] | LCMS m/z [M + 1]: 517 ¹HNMR (400 MHz, CD₃OD) δ 8.74 (s, 1H), 8.19 (s, 1H), 7.17-7.33 (m, 1H), 7.03 (t, 1H), 4.81 (d, 1H), 4.63 (d, 1H), 4.09 (d, 2H), 3.94 (d, 1H), 3.84 (d, 1H), 1.85-2.14 (m, 4H). |
| 5 | 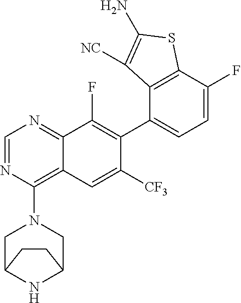 [SMILES: N#Cc1c(N)sc2c(F)ccc(-c3c(C(F)(F)F)cc4c(N5CC6CCC(C5)N6)ncnc4c3F)c12] | LCMS m/z [M + 1]: 517 ¹HNMR (400 MHz, CD₃OD) δ 8.74 (s, 1H), 8.19 (s. 1H), 7.17-7.33 (m, 1H), 7.03 (t, 1H), 4.81 (d, 1H), 4.63 (d, 1H), 4.09 (d, 2H), 3.94 (d, 1H), 3.84 (d, 1H), 1.85-2.14 (m, 4H). |
| 6 | 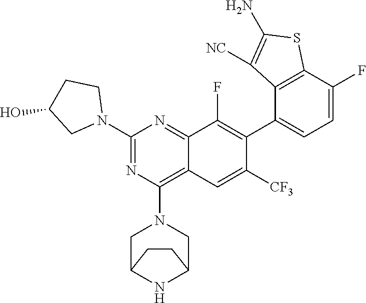 [SMILES: N#Cc1c(N)sc2c(F)ccc(-c3c(C(F)(F)F)cc4c(N5CC6CCC(C5)N6)nc(N5CC[C@@H](O)C5)nc4c3F)c12] | LCMS m/z [M + 1]: 602 ¹HNMR (400 MHz, CD₃OD) δ 8.08 (s, 1H), 7.21-7.25 (m, 1H), 7.04 (t, 1H), 4.56-4.77 (m, 3H), 4.26 (s, 2H), 3.73-4.09 (m, 6H), 2.04-2.24 (m, 6H). |
| 7 | 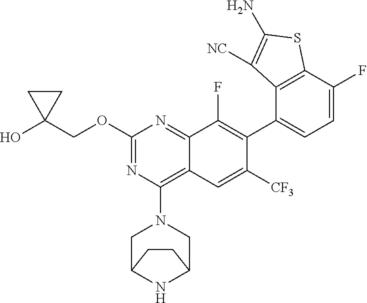 [SMILES: N#Cc1c(N)sc2c(F)ccc(-c3c(C(F)(F)F)cc4c(N5CC6CCC(C5)N6)nc(OCC5(O)CC5)nc4c3F)c12] | LCMS m/z [M + 1]: 602.9 ¹HNMR (400 MHz, CD₃OD) δ 8.48 (brs, 1H), 8.12 (s, 1H), 7.24-7.18 (m, 1H), 7.01 (t, 1H), 4.67 (d, 1H), 4.53 (d, 1H), 4.01-3.88 (m, 3H), 6.83 (d, 1H), 3.74 (d, 1H), 2.02-1.89 (m, 4H), 1.11-0.98 (m, 4H). |
| 8 | 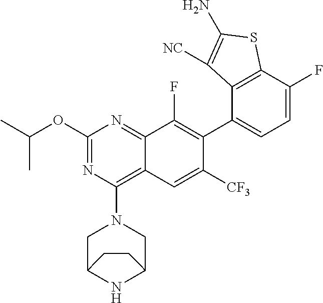 [SMILES: CC(C)Oc1nc(N2CC3CCC(C2)N3)c2cc(C(F)(F)F)c(-c3ccc(F)c4sc(N)c(C#N)c34)c(F)c2n1] | LCMS m/z [M + 1]: 575.0 ¹HNMR (400 MHz, CD₃OD) δ 8.13 (s, 1H), 7.21(t, 1H), 7.02 (t, 1H), 5.48-5.44(m, 1H), 4.76 (d, 1H), 4.64 (d, 1H), 4.25 (s, 2H), 3.98-3.88 (m, 2H), 2.12 (s, 4H), 1.43(d, 6H). |
| 9 | 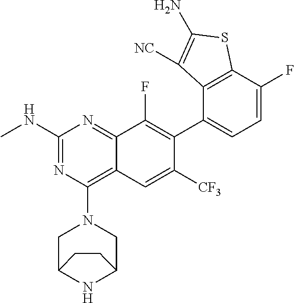 [SMILES: CNc1nc(N2CC3CCC(C2)N3)c2cc(C(F)(F)F)c(-c3ccc(F)c4sc(N)c(C#N)c34)c(F)c2n1] | LCMS m/z [M + 1]: 546.1 ¹HNMR (400 MHz, CD₃OD) δ 8.15 (s, 1H), 7.26-7.23 (m, 1H), 7.07-7.03 (m, 1H), 5.00-4.96 (m, 2H), 4.29 (s, 2H), 4.07 (s, 2H), 3.15 (s, 3H), 2.13-1.99 (m, 4H). |
| 10 | 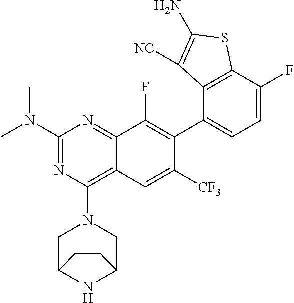 [SMILES: CN(C)c1nc(N2CC3CCC(C2)N3)c2cc(C(F)(F)F)c(-c3ccc(F)c4sc(N)c(C#N)c34)c(F)c2n1] | LCMS m/z [M + 1]: 561.3 ¹HNMR (400 MHz, CD₃OD) δ 8.00 (s, 1H), 7.23-7.20 (m, 1H), 7.05-7.00 (m, 1H), 4.71-4.67 (d, 1H), 4.56-4.52 (d, 1H), 4.25-4.23 (m, 2H), 3.93-3.89 (d, 1H), 3.83-3.80 (d, 1H), 3.33 (s, 6H), 2.19-2.13 (m, 4H). |
| 11 | 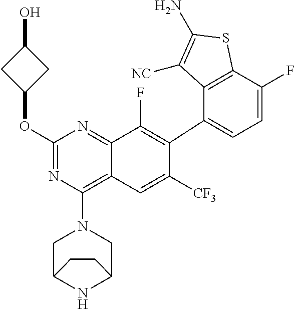 | LCMS m/z [M + 1]: 603.0 ¹HNMR (400 MHz, CD₃OD) δ 8.09 (s, 1H), 7.19-7.22 (m, 1H), 7.01 (t, 1H), 4.92 (d, 1H), 4.65 (t, 2H), 4.52 (d, 1H), 3.97-3.99 (m, 2H), 3.72-3.84 (m, 2H), 2.93-2.96 (m, 2H), 2.10-2.16 (m, 2H), 1.97 (s, 4H). |
| 11a | 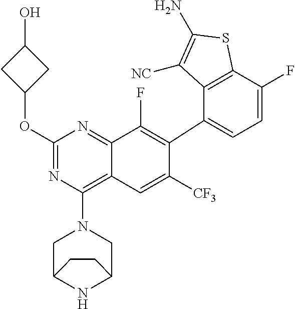 [SMILES: N#Cc1c(N)sc2c(F)ccc(-c3c(C(F)(F)F)cc4c(N5CC6CCC(C5)N6)nc(OC5CC(O)C5)nc4c3F)c12] | |
| 12 | 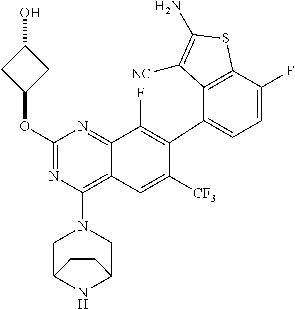 | LCMS m/z [M + 1]: 603.0 ¹HNMR (400 MHz, CD₃OD) δ 8.11 (s, 1H), 7.19-7.23 (m, 1H), 7.02 (t, 1H), 5.48-5.51 (m, 1H), 4.76 (d, 1H), 4.63 (t, 1H), 4.51-4.57 (, 1H), 4.21-4.27 (m, 2H), 3.85-3.96 (m, 2H), 2.45-2.53 (m, 4H), 2.11 (s, 4H). |
| 13 | 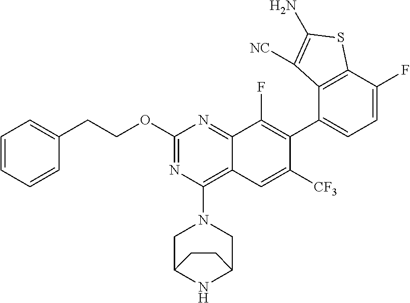 [SMILES: N#Cc1c(N)sc2c(F)ccc(-c3c(C(F)(F)F)cc4c(N5CC6CCC(C5)N6)nc(OCCc5ccccc5)nc4c3F)c12] | LCMS m/z [M + 1]: 637.0 ¹HNMR (400 MHz, CD₃OD) δ 8.09 (s, 1H), 7.15-7.33 (m, 6H), 7.02 (t, 1H), 4.65-4.75 (m, 3H), 4.59 (d, 1H), 4.21 (s, 2H), 3.77-3.92 (m, 3H), 3.13 (t, 2H), 2.07-2.13 (m, 3H). |
| 14 | 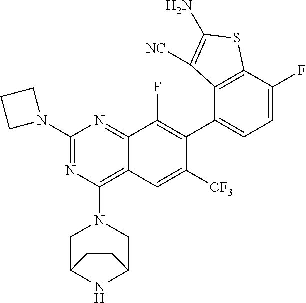 [SMILES: N#Cc1c(N)sc2c(F)ccc(-c3c(C(F)(F)F)cc4c(N5CC6CCC(C5)N6)nc(N5CCC5)nc4c3F)c12] | LCMS m/z [M + 1]: 572.3 ¹HNMR (400 MHz, CD₃OD) δ 8.05 (s, 1H), 7.20-7.24 (m, 1H), 7.03 (t, 1H), 4.77 (d, 1H), 4.66 (d, 1H), 4.32-4.47 (m, 4H), 4.24 (s, 2H), 3.85-3.97 (m, 2H), 2.45-2.53 (m, 2H), 2.11 (s, 4H). |
| 15 | 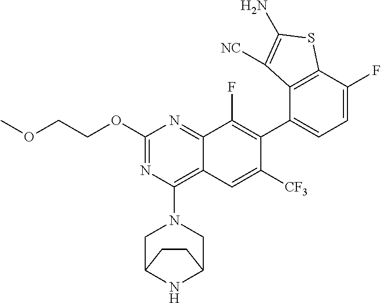 [SMILES: COCCOc1nc(N2CC3CCC(C2)N3)c2cc(C(F)(F)F)c(-c3ccc(F)c4sc(N)c(C#N)c34)c(F)c2n1] | LCMS m/z [M + 1]: 590.6 ¹HNMR (400 MHz, CD₃OD) δ 8.11 (s, 1H), 7.19-7.23 (m, 1H), 7.00-7.04 (m, 1H), 4.73-4.77 (d, 1H), 4.61-4.63 (m, 2H), 4.22 (s, 2H), 3.77- 3.93 (m, 4H), 3.41 (s, 3H), 2.01-2.21 (m, 5H). |
| 16 | 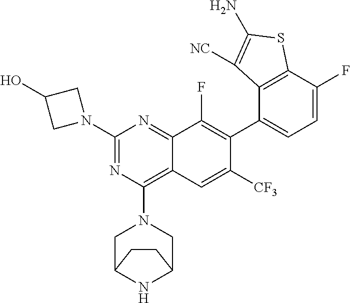 [SMILES: N#Cc1c(N)sc2c(F)ccc(-c3c(C(F)(F)F)cc4c(N5CC6CCC(C5)N6)nc(N5CC(O)C5)nc4c3F)c12] | LCMS m/z [M + 1]: 588 ¹HNMR (400 MHz, CD₃OD) δ 8.111 (s, 1H), 7.21-7.27 (m, 1H), 7.02-7.08 (m, 1H), 4.835 (S, 1H), 4.72-4.78 (m, 2H), 4.58-4.67 (m, 2H), 3.96-4.29 (m, 6H), 2.02-2.17 (m, 4H). |
| 17 | 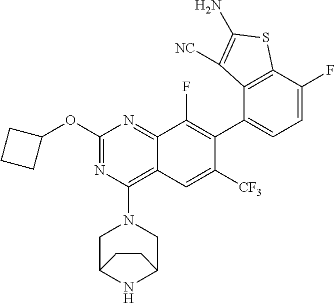 [SMILES: N#Cc1c(N)sc2c(F)ccc(-c3c(C(F)(F)F)cc4c(N5CC6CCC(C5)N6)nc(OC5CCC5)nc4c3F)c12] | LCMS m/z [M + 1]: 587.0 ¹HNMR (400 MHz, CD₃OD) δ 8.10 (s, 1H), 7.17-7.22 (m, 1H), 7.02 (t, 1H), 5.28-5.36 (m, 1H), 4.76 (d, 1H), 4.62 (d, 1H), 4.18-4.27 (m, 2H), 3.93 (d, 1H), 3.84 (d, 1H), 2.47-2.57 (m, 2H), 2.18-2.28 (m, 2H), 2.03-2.17 (m, 4H), 1.85-1.93 (m, 1H), 1.70-1.78 (m, 1H). |
| 18 | 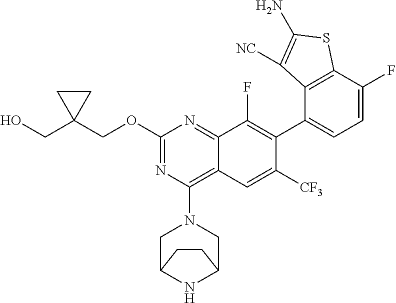 [SMILES: N#Cc1c(N)sc2c(F)ccc(-c3c(C(F)(F)F)cc4c(N5CC6CCC(C5)N6)nc(OCC5(CO)CC5)nc4c3F)c12] | LCMS m/z [M + 1]: 617.17 ¹HNMR (400 MHz, CD₃OD) δ 8.14-8.09 (s, 1H), 7.24-7.18 (m, 1H), 7.05-6.98 (m, 1H), 4.77-4.49 (m, 2H), 4.51-4.37 (s, 2H), 4.09-4.96 (s, 2H), 3.89-3.72 (m, 2H), 3.62-3.51 (s, 2H), 2.75-2.65 (s, 1H), 2.07-1.99 (s, 4H), 0.73-0.65 (m, 2H), 0.63-0.59 (m, 2H). |
| 19 | 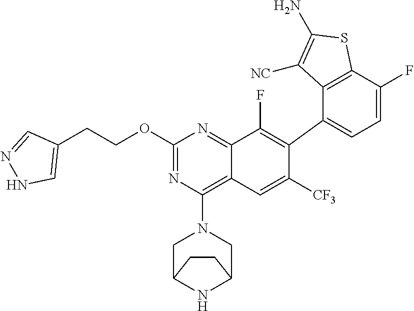 [SMILES: N#Cc1c(N)sc2c(F)ccc(-c3c(C(F)(F)F)cc4c(N5CC6CCC(C5)N6)nc(OCCc5cn[nH]c5)nc4c3F)c12] | LCMS m/z [M + 1]: 627.3 ¹HNMR (400 MHz, CD₃OD): δ 8.45 (brs, 1H), 8.12 (s, 1H), 7.57 (s, 2H), 7.24-7.22 (m, 1H), 7.06-7.02 (m, 1H), 4.70-4.56 (m, 4H), 4.18-4.03 (m, 2H), 3.88-3.79 (m, 2H), 3.05 (t, 2H), 2.13- 1.97 (m, 4H) |
| 20 | 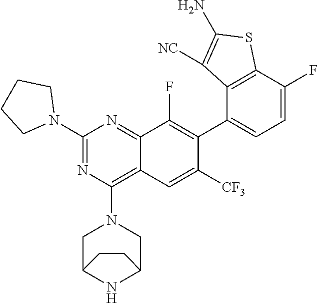 [SMILES: N#Cc1c(N)sc2c(F)ccc(-c3c(C(F)(F)F)cc4c(N5CC6CCC(C5)N6)nc(N5CCCC5)nc4c3F)c12] | LCMS m/z [M + 1]: 586.0 ¹HNMR (400 MHz, CD₃OD) δ 8.02 (s, 1H), 7.20-7.23 (m, 1H), 7.03 (t, 1H), 4.74 (d, 1H), 4.61 (d, 1H), 4.24 (s, 2H), 3.94 (d, 1H), 3.85 (d, 1H), 3.70-3.81 (m, 4H), 2.07-2.13 (m, 8H). |
| 21 | 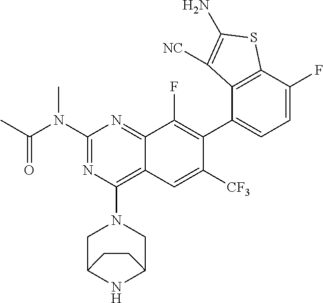 [SMILES: CC(=O)N(C)c1nc(N2CC3CCC(C2)N3)c2cc(C(F)(F)F)c(-c3ccc(F)c4sc(N)c(C#N)c34)c(F)c2n1] | LCMS m/z [M + 1]: 588.15 ¹HNMR (400 MHz, CD₃OD) δ 8.16 (s, 1H), 7.25-7.21 (m, 1H), 7.02 (t, J = 8.8Hz, 1H), 4.65- 4.54 (m, 2H), 4.01 (s, 2H), 3.78 (t, J = 14.4Hz, 2H), 3.51 (s, 3H), 2.50 (s, 3H), 2.05-1.95 (m, 4H). |
| 22 | 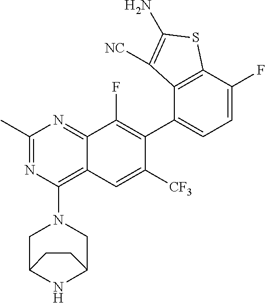 [SMILES: Cc1nc(N2CC3CCC(C2)N3)c2cc(C(F)(F)F)c(-c3ccc(F)c4sc(N)c(C#N)c34)c(F)c2n1] | LCMS m/z [M + 1]: 531 ¹HNMR (400 MHz, CD₃OD) δ 8.46 (s, 0.7H), 8.14 (s, 1H), 7.21-7.25 (m, 1H), 7.00-7.05 (m, 1H), 4.76 (d, 1H), 4.60 (d, 1H), 4.07 (d, 2H), 3.76-3.88 (m, 2H), 2.66 (s, 3H), 1.98-2.03 (m, 4H). |
| 23 | 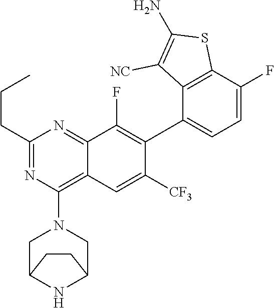 [SMILES: CCCc1nc(N2CC3CCC(C2)N3)c2cc(C(F)(F)F)c(-c3ccc(F)c4sc(N)c(C#N)c34)c(F)c2n1] | LCMS m/z [M + 1]: 559 ¹HNMR (400 MHz, CD₃OD) δ 8.40 (s, 0.3H), 8.14 (s, 1H), 7.22-7.25 (m, 1H), 7.02 (t, 1H), 4.81 (d, 1H), 4.62 (d, 1H), 4.15 (d, 2H), 3.80- 3.94 (m, 2H), 2.90 (t, 2H), 1.86-2.07 (m, 6H), 1.01 (t, 3H). |
| 24 | 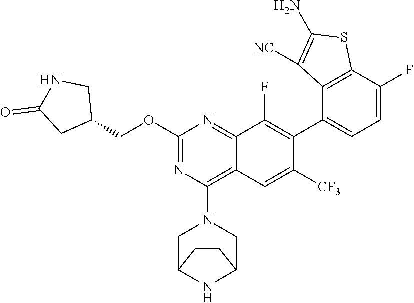 [SMILES: N#Cc1c(N)sc2c(F)ccc(-c3c(C(F)(F)F)cc4c(N5CC6CCC(C5)N6)nc(OC[C@H]5CNC(=O)C5)nc4c3F)c12] | LCMS m/z [M + 1]: 630 ¹HNMR (400 MHz, CD₃OD) δ 8.12 (s, 1H), 7.19-7.23 (m, 1H), 7.02 (t, 1H), 4.45-4.76 (m, 4H), 4.23 (s, 2H), 3.83-3.93 (m, 2H), 3.59-3.87 (m, 1H), 3.02-3.13 (m, 1H), 2.52-2.58 (m, 1H), 2.27-2.33 (m, 1H), 2.12 (s, 4H), 1.28-1.66 (m, 1H). |
| 24a | 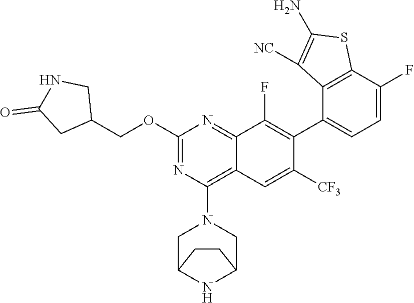 [SMILES: N#Cc1c(N)sc2c(F)ccc(-c3c(C(F)(F)F)cc4c(N5CC6CCC(C5)N6)nc(OCC5CNC(=O)C5)nc4c3F)c12] | |
| 25 | 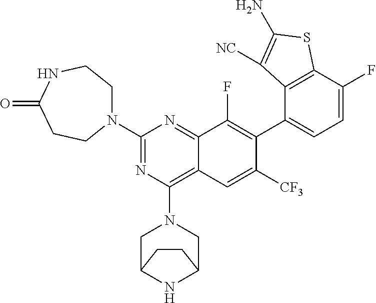 [SMILES: N#Cc1c(N)sc2c(F)ccc(-c3c(C(F)(F)F)cc4c(N5CC6CCC(C5)N6)nc(N5CCNC(=O)CC5)nc4c3F)c12] | LCMS m/z [M + 1]: 630.3 ¹HNMR (400 MHz, CD₃OD) δ 7.95 (s, 1H), 7.19 (m, 1H), 7.00 (s, 1H), 4.57 (d, 1H), 4.43 (d, 1H), 4.10-4.24 (m, 6H), 3.80 (d, 1H), 3.71 (d, 1H), 3.39-3.44 (m, 2H), 2.73 (t, 2H), 2.11-2.22 (m, 4H). |
| 26 | 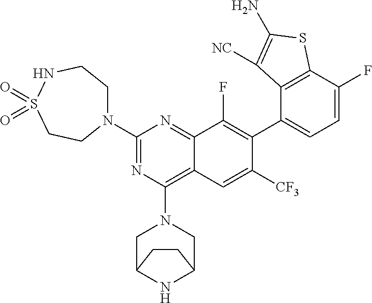 [SMILES: N#Cc1c(N)sc2c(F)ccc(-c3c(C(F)(F)F)cc4c(N5CC6CCC(C5)N6)nc(N5CCNS(=O)(=O)CC5)nc4c3F)c12] | LCMS m/z [M + 1]: 666.1 ¹HNMR (400 MHz, CD₃OD) δ 7.977 (s, 1H), 7.16-7.22 (m, 1H), 6.97-7.03 (m, 1H), 4.43-4.59 (m, 2H), 4.08-4.26 (m, 6H), 3.73-3.856 (m, 2H), 3.457-3.582 (m, 2H), 3.318-3.418 (m, 2H), 2.107-2.218 (m, 4H). |
| 27 | 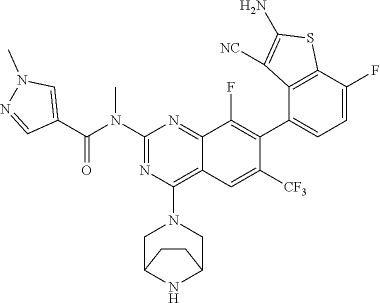 [SMILES: CN(C(=O)c1cnn(C)c1)c1nc(N2CC3CCC(C2)N3)c2cc(C(F)(F)F)c(-c3ccc(F)c4sc(N)c(C#N)c34)c(F)c2n1] | LCMS m/z [M + 1]: 654.1 ¹HNMR (400 MHz, CD₃OD) δ 8.14 (s, 1H), 7.88 (s, 1H), 7.74 (s, 1H), 7.45 (s, 1H), 7.25- 7.22 (m, 1H), 7.06-7.01 (m, 1H), 4.44-4.39 (m, 2H), 4.10 (s, 2H), 3.84-3.77 (m, 5H), 3.61 (s, 3H), 1.99-1.95 (m, 2H), 1.82-1.70 (m, 2H). |
| 28 | 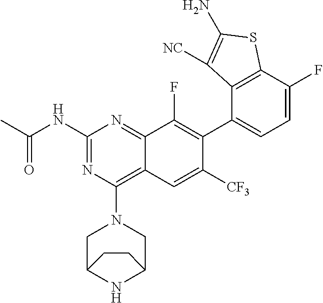 [SMILES: CC(=O)Nc1nc(N2CC3CCC(C2)N3)c2cc(C(F)(F)F)c(-c3ccc(F)c4sc(N)c(C#N)c34)c(F)c2n1] | LCMS m/z [M + 1]: 574 ¹HNMR (400 MHz, CD₃OD) δ 8.16 (s, 1H), 7.22-7.25 (m, 1H), 7.01-7.05 (m, 1H), 4.89 (s, 1H), 4.71 (d, 1H), 4.23 (s, 2H), 4.03 (d, 1H), 3.93 (d, 1H), 2.38 (s, 3H), 2.01-2.15 (m, 4H). |
| 29 | 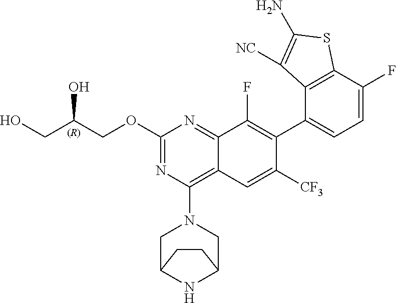 | LCMS m/z [M + 1]: 607.1 ¹HNMR (400 MHz, CD₃OD) δ 8.33 (s, 1H), 8.11 (s, 1H), 7.24-7.17 (m, 1H), 7.05-6.98 (m, 1H), 4.78-4.70 (m, 1H), 4.65-4.53 (m, 2H), 4.51-4.42 (m, 1H), 4.15 (s, 2H), 4.05-3.98 (m, 1H), 3.93-3.79 (m, 2H), 3.72-3.60 (m, 2H), 2.08 (s, 4H). |
| 29a | 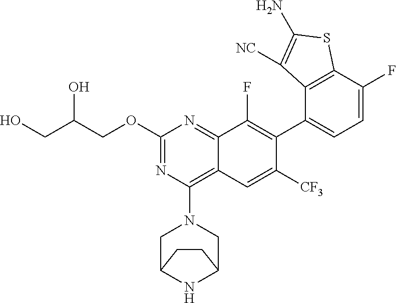 [SMILES: N#Cc1c(N)sc2c(F)ccc(-c3c(C(F)(F)F)cc4c(N5CC6CCC(C5)N6)nc(OCC(O)CO)nc4c3F)c12] | |
| 30 | 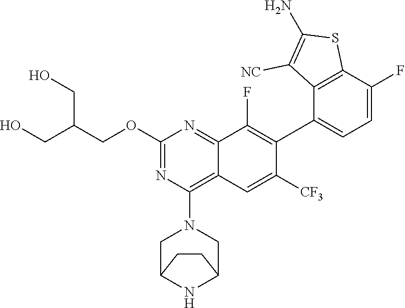 [SMILES: N#Cc1c(N)sc2c(F)ccc(-c3c(C(F)(F)F)cc4c(N5CC6CCC(C5)N6)nc(OCC(CO)CO)nc4c3F)c12] | LCMS m/z [M + 1]: 621.1 ¹HNMR (400 MHz, CD₃OD) δ 8.11 (s, 1H), 7.23-7.19 (m, 1H), 7.04-6.99 (m, 1H), 4.73-4.70 (m, 1H), 4.61-4.55 (m, 3H), 4.02 (s, 2H), 3.87- 3.70 (m, 6H), 2.19-2.16 (m, 1H), 2.02 (s, 4H). |
| 31 | 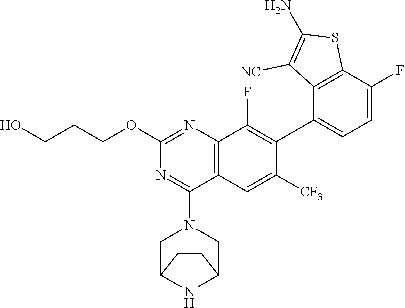 [SMILES: N#Cc1c(N)sc2c(F)ccc(-c3c(C(F)(F)F)cc4c(N5CC6CCC(C5)N6)nc(OCCCO)nc4c3F)c12] | LCMS m/z [M + 1]: 591.15 ¹HNMR (400 MHz, CD₃OD) δ 8.10 (s, 1H), 7.24-7.17 (m, 1H), 7.05-6.97 (m, 1H), 4.73-4.65 (m, 1H), 4.61-4.49 (m, 3H), 4.08-3.98 (m, 2H), 3.90-3.71 (m, 4H), 2.13-1.90 (m, 6H). |
| 32 | 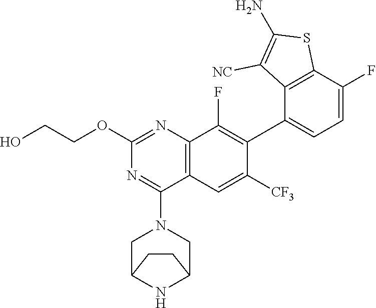 [SMILES: N#Cc1c(N)sc2c(F)ccc(-c3c(C(F)(F)F)cc4c(N5CC6CCC(C5)N6)nc(OCCO)nc4c3F)c12] | LCMS m/z [M + 1]: 577.1 ¹HNMR (400 MHz, CD₃OD) δ 8.14 (s, 1H), 7.25-7.16 (m, 1H), 7.06-6.93 (m, 1H), 4.73-4.63 (m, 1H), 4.62-4.43 (m, 3H), 4.07-3.69 (m, 6H), 2.97-2.26 (m, 3H), 2.12-1.87 (m, 4H). |
| 33 | 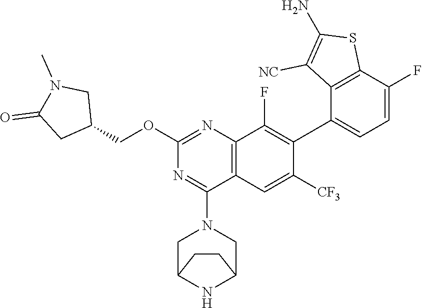 [SMILES: CN1C[C@H](COc2nc(N3CC4CCC(C3)N4)c3cc(C(F)(F)F)c(-c4ccc(F)c5sc(N)c(C#N)c45)c(F)c3n2)CC1=O] | LCMS m/z [M + 1]: 644 ¹HNMR (400 MHz, CD₃OD) δ 8.11 (s, 1H), 7.19-7.22 (m, 1H), 7.00 (t, 1H), 4.40-4.57 (m, 4H), 3.63-3.72 (m, 5H), 3.39-3.47 (m, 1H), 2.90-2.96 (m, 1H), 2.84 (s, 3H), 2.58-2.65 (m, 1H), 2.33-2.39 (m, 1H), 1.78-1.82 (m, 4H). |
| 33a | 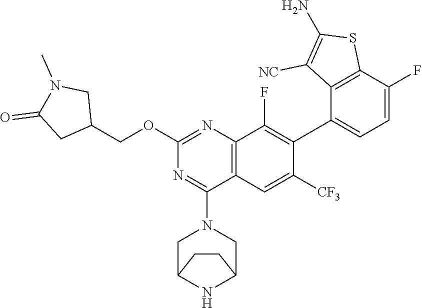 [SMILES: CN1CC(COc2nc(N3CC4CCC(C3)N4)c3cc(C(F)(F)F)c(-c4ccc(F)c5sc(N)c(C#N)c45)c(F)c3n2)CC1=O] | |
| 34 | 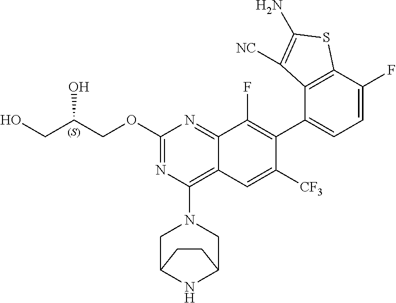 | LCMS m/z [M + 1]: 607.0 ¹HNMR (400 MHz, CD₃OD) δ 8.11 (s, 1H), 7.25-7.17 (m, 1H), 7.06-6.97 (m, 1H), 4.74-4.63 (m, 1H), 4.60-4.52 (m, 2H), 4.50-4.40 (m, 1H), 4.09-3.51 (m, 7H), 2.81 (s, 2H), 2.08-1.83 (m, 4H). |
| 35 | 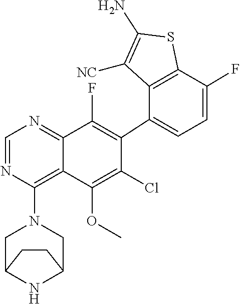 [SMILES: COc1c(Cl)c(-c2ccc(F)c3sc(N)c(C#N)c23)c(F)c2ncnc(N3CC4CCC(C3)N4)c12] | LCMS m/z [M + 1]: 513.0 ¹HNMR (400 MHz, CD₃OD) δ 8.45 (s, 1H), 7.22-7.25 (m, 1H), 7.02-7.06 (m, 1H), 3.73 (s, 3H), 3.47-3.59 (m, 4H), 2.79 (s, 2H), 1.71 (s, 4H). |
| 36 | 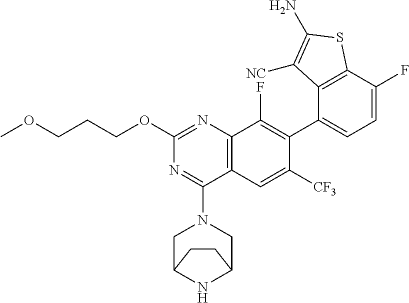 [SMILES: COCCCOc1nc(N2CC3CCC(C2)N3)c2cc(C(F)(F)F)c(-c3ccc(F)c4sc(N)c(C#N)c34)c(F)c2n1] | LCMS m/z [M + 1]: 605.17 ¹HNMR (400 MHz, CD₃OD) δ 8.13 (s, 1H), 7.30-7.20 (m, 1H), 7.09-6.99 (m, 1H), 4.67-4.40 (m, 4H), 3.78-3.58 (m, 6H), 3.43-3.36 (m, 3H), 2.15-2.05 (m, 2H), 1.93-1.74 (m, 4H). |
| 37 | 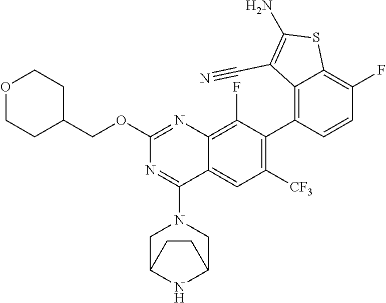 [SMILES: N#Cc1c(N)sc2c(F)ccc(-c3c(C(F)(F)F)cc4c(N5CC6CCC(C5)N6)nc(OCC5CCOCC5)nc4c3F)c12] | LCMS m/z [M + 1]: 631.2 ¹HNMR (400 MHz, CD₃OD) δ 8.10 (s, 1H), 7.19-7.22 (m, 1H), 7.00 (t, 1H), 4.56 (t, 1H), 4.44 (d, 1H), 4.31 (d, 2H), 3.96-4.00 (m, 2H), 3.62-3.72 (m, 4H), 3.46 (t, 2H), 2.10-2.16 (m, 1H), 1.77-1.80 (m, 6H), 1.41-1.52 (m, 2H). |
| 38 | 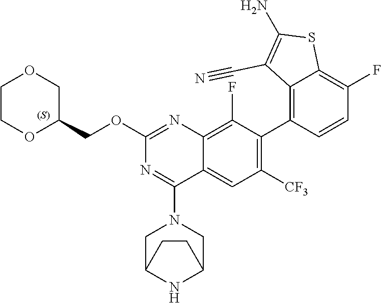 | LCMS m/z [M + 1]: 633.0 ¹HNMR (400 MHz, CD₃OD) δ 8.10 (s, 1H), 7.19-7.22 (m, 1H), 7.00 (t, 1H), 4.56 (t, 1H), 4.40-4.46 (m, 3H), 3.97-9-3.98 (m, 1H), 3.88- 3.92 (m, 1H), 3.79-3.83 (m, 1H), 3.69-3.76 (m, 3H), 3.52-3.65 (m, 5H), 1.78-1.80 (m, 4H). |
| 38a | 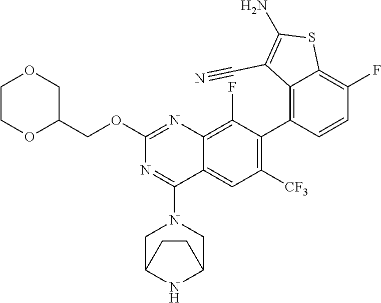 [SMILES: N#Cc1c(N)sc2c(F)ccc(-c3c(C(F)(F)F)cc4c(N5CC6CCC(C5)N6)nc(OCC5COCCO5)nc4c3F)c12] | |
| 39 | 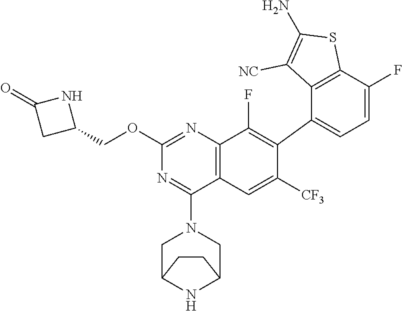 [SMILES: N#Cc1c(N)sc2c(F)ccc(-c3c(C(F)(F)F)cc4c(N5CC6CCC(C5)N6)nc(OC[C@@H]5CC(=O)N5)nc4c3F)c12] | LCMS m/z [M + 1]: 616.2 ¹HNMR (400 MHz, CD₃OD) δ 7.99 (s, 1H), 7.18-7.21 (m, 1H), 7.01 (t, 1H), 4.64-4.69 (m, 2H), 4.36-4.40 (m, 1H), 4.21 (s, 2H), 3.70-3.89 (m, 4H), 3.01-3.07 (m, 1H), 2.67 (d, 1H), 2.13 (s, 4H). |
| 39a | 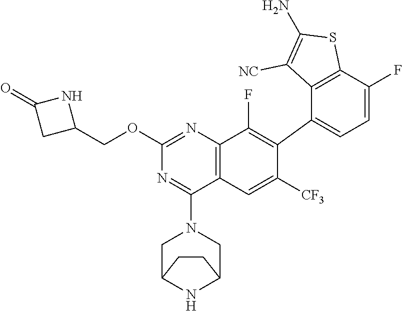 [SMILES: N#Cc1c(N)sc2c(F)ccc(-c3c(C(F)(F)F)cc4c(N5CC6CCC(C5)N6)nc(OCC5CC(=O)N5)nc4c3F)c12] | |
| 40 | 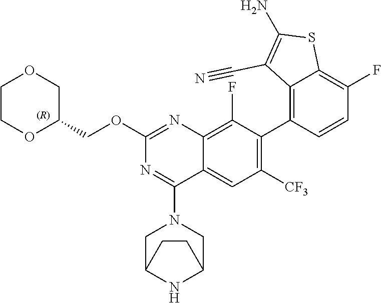 | LCMS m/z [M + 1]: 633.3 ¹HNMR (400 MHz, CD₃OD) δ 8.10 (s, 1H), 7.19-7.22 (m, 1H), 6.98-7.02 (m, 1H), 4.754- 4.57 (m, 1H), 4.37-4.48 (m, 3H), 3.95-4.01 (m, 1H), 3.488-3.92 (m, 1H), 3.69-3.82 (m, 4H), 3.51-3.64 (m, 5H), 1.75-1.87 (m, 4H). |
| 41 | 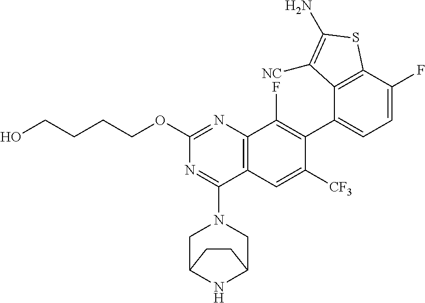 [SMILES: N#Cc1c(N)sc2c(F)ccc(-c3c(C(F)(F)F)cc4c(N5CC6CCC(C5)N6)nc(OCCCCO)nc4c3F)c12] | LCMS m/z [M + 1]: 605.0 ¹HNMR (400 MHz, CD₃OD) δ 8.10 (s, 1H), (m, 1H), 7.04-6.97 (m, 1H), 4.62-4.58 (m, 1H), 4.53-4.42 (m, 3H), 3.85-3.72 (m, 3H), 3.71-3.66 (m, 1H), 3.65-3.59 (m, 2H), 1.98-1.80 (m, 6H), 1.76-1.68 (m, 2H). |
| 42 | 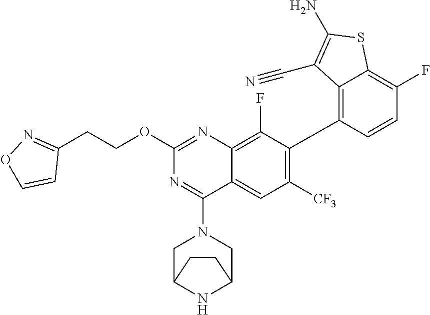 [SMILES: N#Cc1c(N)sc2c(F)ccc(-c3c(C(F)(F)F)cc4c(N5CC6CCC(C5)N6)nc(OCCc5ccon5)nc4c3F)c12] | LCMS m/z [M + 1]: 628.0 ¹HNMR (400 MHz, CD₃OD) δ 8.57 (s, 1H), 8.11 (s, 1H), 7.22-7.19 (m, 1H), 7.03-6.99 (m, 1H), 6.55-6.54 (m, 1H), 4.79-4.76 (m, 2H), 4.69-4.65 (m, 1H), 4.56-4.53 (m, 1H), 3.98 (s, 2H), 3.87-3.76 (m, 2H), 3.26-3.23 (m, 2H), 1.98 (s, 4H). |
| 43 | 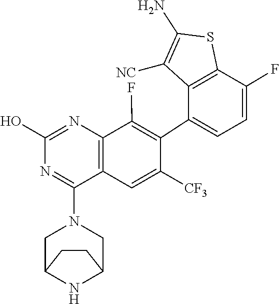 [SMILES: N#Cc1c(N)sc2c(F)ccc(-c3c(C(F)(F)F)cc4c(N5CC6CCC(C5)N6)nc(O)nc4c3F)c12] | LCMS m/z [M + 1]: 533.0 ¹H NMR (400 MHz, CD₃OD) δ 8.05 (s, 1H), 7.24-7.21 (m, 1H), 7.03-6.99 (m, 1H), 4.60-4.53 (m, 1H), 4.46-4.39 (m, 1H), 3.67-3.40 (m, 4H), 1.82-1.72 (m, 3H), 1.57 (s, 1H). |
| 44 | 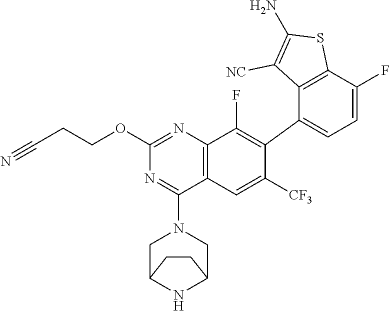 [SMILES: N#CCCOc1nc(N2CC3CCC(C2)N3)c2cc(C(F)(F)F)c(-c3ccc(F)c4sc(N)c(C#N)c34)c(F)c2n1] | LCMS m/z [M + 1]: 586.0 ¹H NMR (400 MHz, CD₃OD) δ 8.19 (s, 1H), 7.21 (s, 1H), 7.03-6.98 (m, 1H), 4.65-4.40 (m, 4H), 3.75-3.65 (m, 3H), 3.51-3.40 (m, 1H), 3.02-2.99 (m, 2H), 1.83 (s, 3H), 1.60-1.53 (m, 1H). |
| 45 | 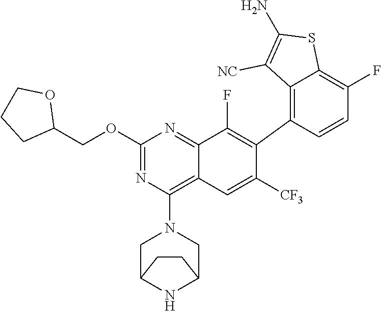 [SMILES: N#Cc1c(N)sc2c(F)ccc(-c3c(C(F)(F)F)cc4c(N5CC6CCC(C5)N6)nc(OCC5CCCO5)nc4c3F)c12] | LCMS m/z [M + 1]: 617.0 ¹HNMR (400 MHz, CD₃OD) δ 8.12 (s, 1H), 7.24-7.21 (m, 1H), 7.06-7.01 (m, 1H), 4.76-4.72 (m, 1H), 4.62-4.59 (m, 1H), 4.53-4.45 (m, 2H), 4.34-4.31 (m, 1H), 4.17 (s, 2H), 3.95-3.79 (m, 4H), 2.14-1.82 (m, 8H). |
| 46 | 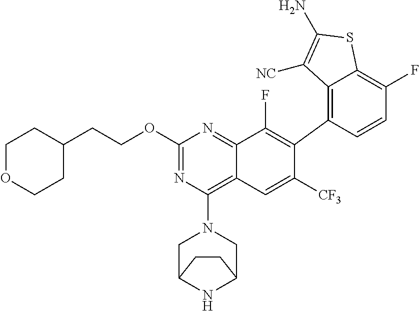 [SMILES: N#Cc1c(N)sc2c(F)ccc(-c3c(C(F)(F)F)cc4c(N5CC6CCC(C5)N6)nc(OCCC5CCOCC5)nc4c3F)c12] | LCMS m/z [M + 1]: 645.20 ¹HNMR (400 MHz, CD₃OD) δ 8.11 (s, 1H), 7.25-7.18 (m, 1H), 7.05-6.98 (m, 1H), 4.61-4.42 (m, 4H), 3.99-3.89 (m, 2H), 3.77-3.61 (m, 4H), 3.48-3.39 (m, 2H), 1.91-1.66 (m, 9H), 1.41-1.32 (m, 2H). |
| 47 | 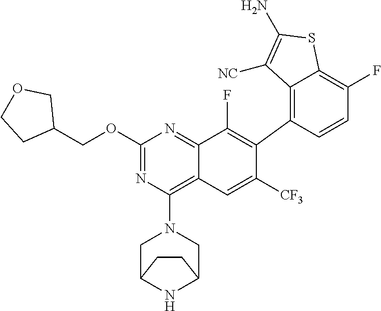 [SMILES: N#Cc1c(N)sc2c(F)ccc(-c3c(C(F)(F)F)cc4c(N5CC6CCC(C5)N6)nc(OCC5CCOC5)nc4c3F)c12] | LCMS m/z [M + 1]: 617.0 ¹HNMR (400 MHz, CD₃OD) δ 8.11 (s, 1H), 7.23-7.20 (m, 1H), 7.03-6.99 (m, 1H), 4.58-4.55 (m, 1H), 4.46-4.43 (m, 2H), 4.38-4.34 (m, 1H), 3.94-3.89 (m, 2H), 3.81-3.63 (m, 6H), 2.82-2.77 (m, 1H), 2.19-2.10 (m, 1H), 1.85-1.77 (m, 5H). |
| 48 | 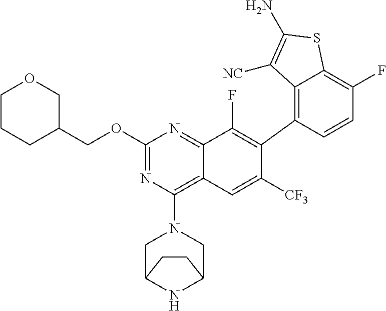 [SMILES: N#Cc1c(N)sc2c(F)ccc(-c3c(C(F)(F)F)cc4c(N5CC6CCC(C5)N6)nc(OCC5CCCOC5)nc4c3F)c12] | LCMS m/z [M + 1]: 631.18 ¹HNMR (400 MHz, CD₃OD) δ 8.12 (s, 1H), 7.25-7.20 (m, 1H), 7.05-6.99 (m, 1H), 4.62-4.53 (m, 1H), 4.49-4.36 (m, 2H), 4.34-4.28 (m, 1H), 4.08-4.01 (m, 1H), 3.89-3.82 (m, 1H), 3.76-3.61 (m, 4H), 3.53-3.40 (m, 2H), 2.22-2.13 (m, 1H), 1.99-1.92 (m, 1H), 1.88-1.76 (m, 4H), 1.75-1.63 (m, 2H), 1.57-1.47 (m, 1H). |
| 49 | 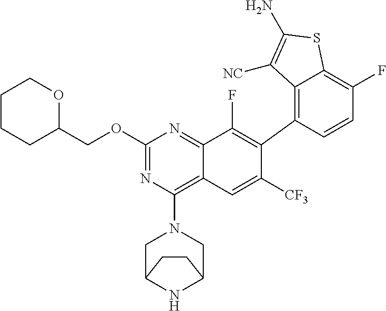 [SMILES: N#Cc1c(N)sc2c(F)ccc(-c3c(C(F)(F)F)cc4c(N5CC6CCC(C5)N6)nc(OCC5CCCCO5)nc4c3F)c12] | LCMS m/z [M + 1]: 631.0 ¹HNMR (400 MHz, CD₃OD) δ 8.10 (s, 1H), 7.23-7.17 (m, 1H), 7.03-6.97 (m, 1H), 4.60-4.53 (m, 1H), 4.46-4.33 (m, 3H), 4.00-3.94 (m, 1H), 3.81-3.57 (m, 5H), 3.53-3.48 (m, 1H), 1.97-1.68 (m, 6H), 1.63-1.46 (m, 4H). |
| 50 | 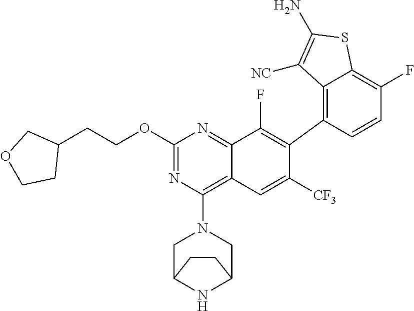 [SMILES: N#Cc1c(N)sc2c(F)ccc(-c3c(C(F)(F)F)cc4c(N5CC6CCC(C5)N6)nc(OCCC5CCOC5)nc4c3F)c12] | LCMS m/z [M + 1]: 631.2 ¹HNMR (400 MHz, CD₃OD) δ 8.10 (s, 1H), 7.23-7.19 (m, 1H), 7.03-6.98 (m, 1H), 4.57-4.43 (m, 4H), 3.98-3.94 (m, 1H), 3.89-3.84 (m, 1H), 3.78-3.63 (m, 5H), 3.49-3.42 (m, 1H), 2.47-2.40 (m, 1H), 2.19-2.13 (m, 1H), 1.99-1.88 (m, 2H), 1.82-1.79 (m, 4H), 1.70-1.61 (m, 1H). |
| 51 | 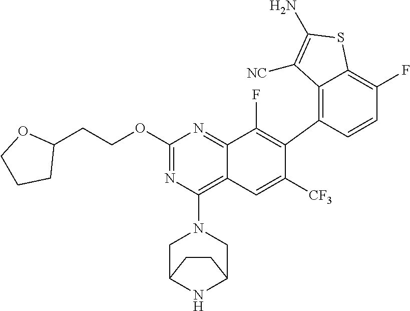 [SMILES: N#Cc1c(N)sc2c(F)ccc(-c3c(C(F)(F)F)cc4c(N5CC6CCC(C5)N6)nc(OCCC5CCCO5)nc4c3F)c12] | LCMS m/z [M + 1]: 631.18 ¹HNMR (400 MHz, CD₃OD) δ 8.11 (s, 1H), 7.26-7.15 (m, 1H), 7.06-6.95 (m, 1H), 4.77-4.68 (m, 1H), 4.64-4.49 (m, 3H), 4.15-4.00 (m, 3H), 3.98-3.80 (m, 3H), 3.78-3.68 (m, 1H), 2.14-1.86 (m, 9H), 1.66-1.53 (m, 1H). |
| 52 | 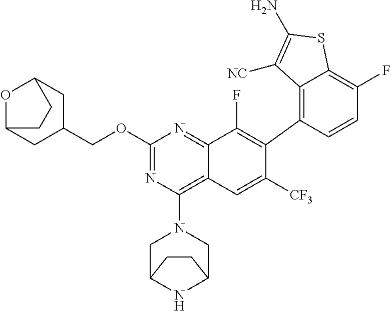 [SMILES: N#Cc1c(N)sc2c(F)ccc(-c3c(C(F)(F)F)cc4c(N5CC6CCC(C5)N6)nc(OCC5CC6CCC(C5)O6)nc4c3F)c12] | LCMS m/z [M + 1]: 657.2 ¹HNMR (400 MHz, CD₃OD) δ 8.09 (s, 1H), 7.24-7.17 (m, 1H), 7.03-6.96 (m, 1H), 4.60-4.53 (m, 1H), 4.49-4.35 (m, 3H), 4.29-4.20 (m, 2H), 3.77-3.59 (m, 4H), 2.43-2.33 (m, 1H), 2.04-1.76 (m, 8H), 1.73-1.65 (m, 2H), 1.61-1.51 (m, 2H). |
| 53 | 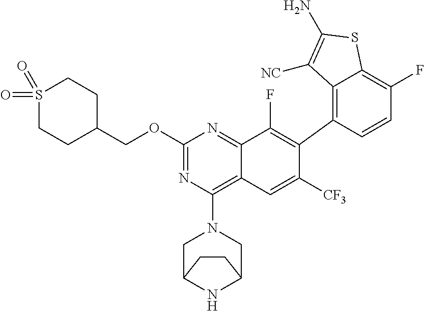 [SMILES: N#Cc1c(N)sc2c(F)ccc(-c3c(C(F)(F)F)cc4c(N5CC6CCC(C5)N6)nc(OCC5CCS(=O)(=O)CC5)nc4c3F)c12] | LCMS m/z [M + 1]: 679.1 ¹HNMR (400 MHz, CD₃OD) δ 8.12 (s, 1H), 7.23-7.20 (m, 1H), 7.04-6.99 (m, 1H), 4.57-4.54 (m, 1H), 4.47-4.44 (m, 1H), 4.39-4.37 (m, 2H), 3.73-3.63 (m, 4H), 3.20-3.08 (m, 4H), 2.32-2.28 (m, 2H), 2.19 (m, 1H), 2.02-1.92 (m, 2H), 1.81- 1.79 (m, 4H). |
| 54 | 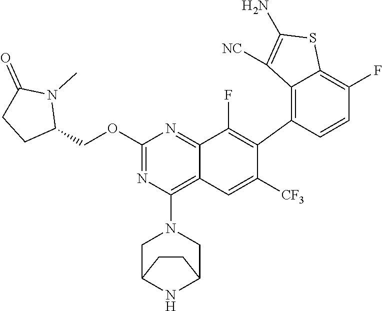 [SMILES: CN1C(=O)CC[C@H]1COc1nc(N2CC3CCC(C2)N3)c2cc(C(F)(F)F)c(-c3ccc(F)c4sc(N)c(C#N)c34)c(F)c2n1] | LCMS m/z [M + 1]: 644.2 ¹HNMR (400 MHz, CD₃OD) δ 8.12 (s, 1H), 7.23-7.19 (m, 1H), 7.03-6.98 (m, 1H), 4.76-4.73 (m, 1H), 4.59-4.44 (m, 3H), 4.03-4.00 (m, 1H), 3.74-3.58 (m, 4H), 2.91 (s, 3H), 2.63-2.57 (m, 1H), 2.36- 2.26 (m, 2H), 2.07-2.02 (m, 1H), 1.84-1.79 (m, 4H). |
| 54a | 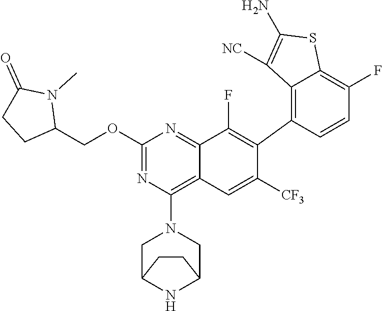 [SMILES: CN1C(=O)CCC1COc1nc(N2CC3CCC(C2)N3)c2cc(C(F)(F)F)c(-c3ccc(F)c4sc(N)c(C#N)c34)c(F)c2n1] | |
| 55 | 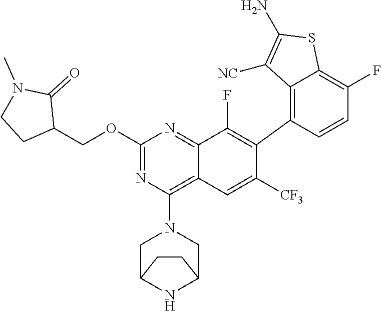 [SMILES: CN1CCC(COc2nc(N3CC4CCC(C3)N4)c3cc(C(F)(F)F)c(-c4ccc(F)c5sc(N)c(C#N)c45)c(F)c3n2)C1=O] | LCMS m/z [M + 1]: 644.0 ¹HNMR (400 MHz, CD₃OD) δ 8.11 (s, 1H), 7.25-7.18 (m, 1H), 7.04-6.98 (m, 1H), 4.71-4.58 (m, 3H), 4.53-4.44 (m, 1H), 3.80-3.59 (m, 4H), 3.54-3.41 (m, 2H), 3.01-2.93 (m, 1H), 2.87 (s, 3H), 2.37-2.28 (m, 1H), 2.22-2.14 (m, 1H), 1.92-1.74 (m, 4H). |
| 56 | 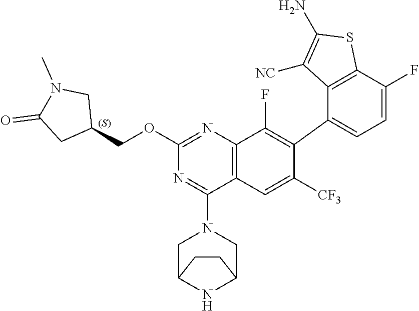 | LCMS m/z [M + 1]: 644.2 ¹H NMR (400 MHz, CD₃OD) δ 8.11 (s, 1H), 7.21-7.19 (m, 1H), 7.03-6.98 (m, 1H), 4.60-4.40 (m, 4H), 3.78-3.58 (m, 5H), 3.44-3.41 (m, 1H), 2.95-2.93 (m, 1H), 2.85 (s, 3H), 2.65-2.59 (m, 1H), 2.93-2.34 (m, 1H), 1.90-1.79 (m, 4H). |
| 56a | 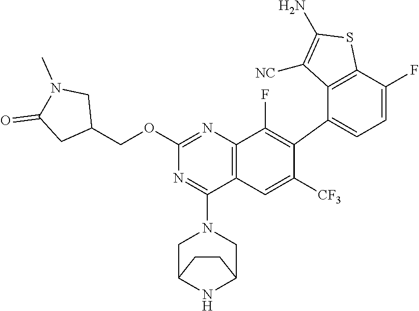 [SMILES: CN1CC(COc2nc(N3CC4CCC(C3)N4)c3cc(C(F)(F)F)c(-c4ccc(F)c5sc(N)c(C#N)c45)c(F)c3n2)CC1=O] | |
| 57 | 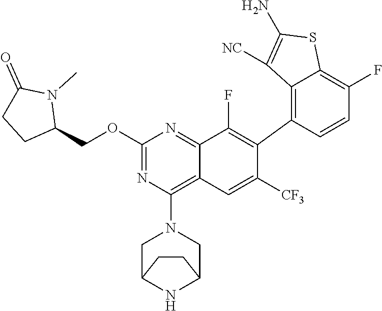 [SMILES: CN1C(=O)CC[C@@H]1COc1nc(N2CC3CCC(C2)N3)c2cc(C(F)(F)F)c(-c3ccc(F)c4sc(N)c(C#N)c34)c(F)c2n1] | LCMS m/z [M + 1]: 644.0 ¹H NMR (400 MHz, CD₃OD) δ 8.13 (s, 1H), 7.24-7.21 (m, 1H), 7.05-7.00 (m, 1H), 4.80-4.76 (m, 1H), 4.64-4.59 (m, 1H), 4.55-4.51 (m, 2H), 4.03-4.02 (m, 1H), 3.79-3.68 (m, 4H), 2.93 (s, 3H), 2.63-2.58 (m, 1H), 2.37-2.30 (m, 2H), 2.08-1.90 (m, 5H). |
| 58 | 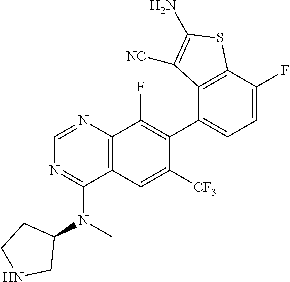 [SMILES: CN(c1ncnc2c(F)c(-c3ccc(F)c4sc(N)c(C#N)c34)c(C(F)(F)F)cc12)[C@@H]1CCNC1] | LCMS m/z [M + 1]: 505 ¹HNMR (400 MHz, CD₃OD) δ 8.76 (s, 1H), 8.41 (s, 1H), 7.22-7.29 (m, 1H), 7.04 (t, 1H), 5.05-5.18 (m, 1H), 3.61-3.82 (m, 3H), 3.59 (d, 3H), 3.33-3.42 (m, 1H), 2.53-2.67 (m, 1H), 2.39-2.53 (m, 1H). |
| 58a | 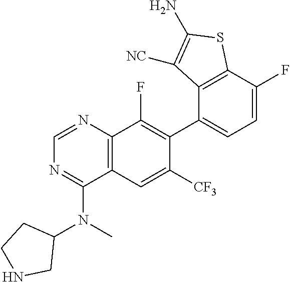 [SMILES: CN(c1ncnc2c(F)c(-c3ccc(F)c4sc(N)c(C#N)c34)c(C(F)(F)F)cc12)C1CCNC1] | |
| 59 | 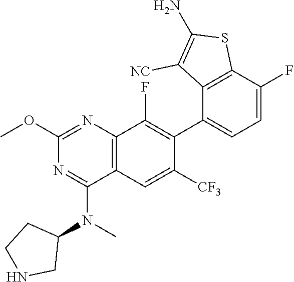 [SMILES: COc1nc(N(C)[C@@H]2CCNC2)c2cc(C(F)(F)F)c(-c3ccc(F)c4sc(N)c(C#N)c34)c(F)c2n1] | LCMS m/z [M + 1]: 535.0 ¹HNMR (400 MHz, CD₃OD) δ 8.54 (s, 1H), 8.29 (s, 1H), 7.20-7.23 (m, 1H), 6.99-7.04 (t, 1H), 5.14-5.16 (m, 1H), 4.07 (s, 3H) 3.60- 3.72 (m, 2H), 3.49 (s, 3H), 2.35-3.43 (m, 1H), 2.33-2.51 (m, 3H). |
| 59a | 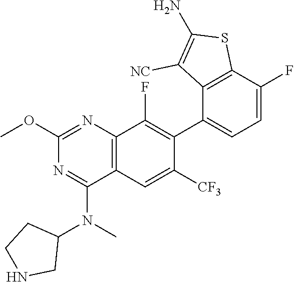 [SMILES: COc1nc(N(C)C2CCNC2)c2cc(C(F)(F)F)c(-c3ccc(F)c4sc(N)c(C#N)c34)c(F)c2n1] | |
| 60 | 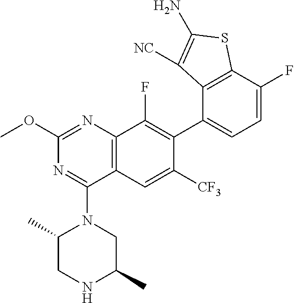 [SMILES: COc1nc(N2C[C@@H](C)NC[C@@H]2C)c2cc(C(F)(F)F)c(-c3ccc(F)c4sc(N)c(C#N)c34)c(F)c2n1] | LCMS m/z [M + 1]: 549 ¹HNMR (400 MHz, CD₃OD) δ 8.13-8.25 (m, 1H), 7.14-7.27 (m, 1H), 6.98-7.07 (m, 1H), 4.44-4.54 (m, 1H), 4.114 (s, 3H), 3.75-3.97 (m, 3H), 3.58-3.67 (m, 1H), 1.43-1.65 (m, 3H), 1.41-1.44 (m, 1H), 1.373 (s, 3H). |
| 60a | 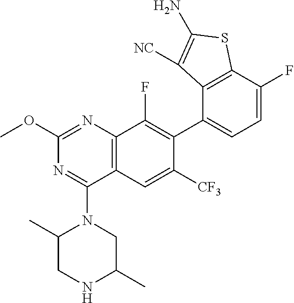 [SMILES: COc1nc(N2CC(C)NCC2C)c2cc(C(F)(F)F)c(-c3ccc(F)c4sc(N)c(C#N)c34)c(F)c2n1] | |
| 61 | 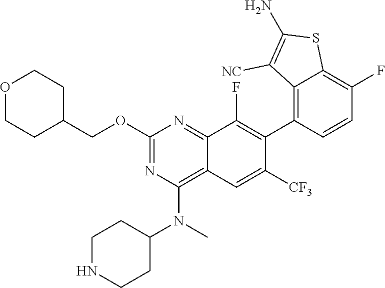 [SMILES: CN(c1nc(OCC2CCOCC2)nc2c(F)c(-c3ccc(F)c4sc(N)c(C#N)c34)c(C(F)(F)F)cc12)C1CCNCC1] | LCMS m/z [M + 1]: 633.0 ¹HNMR (400 MHz, CD₃OD) δ 8.25 (s, 1H), 7.24-7.18 (m, 1H), 7.00 (t, J = 8.4 Hz, 1H), 4.78- 4.69 (m, H), 4.32 (d, J = 11.2 Hz, 2H), 4.02-3.94 (m, 2H), 3.50-3.41 (m, 2H), 3.39 (s, 3H), 3.28- 3.17 (m, 2H), 2.87-2.74 (m, 2H), 2.19-2.09 (m, 1H), 2.06-1.92 (m, 4H), 1.81-1.73 (m, 2H), 1.51- 1.39 (m, 2H). |
| 62 | 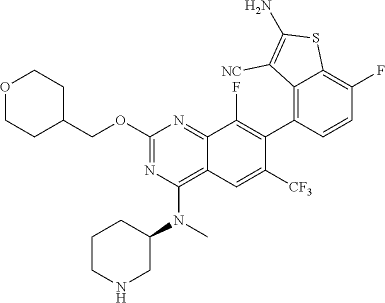 [SMILES: CN(c1nc(OCC2CCOCC2)nc2c(F)c(-c3ccc(F)c4sc(N)c(C#N)c34)c(C(F)(F)F)cc12)[C@@H]1CCCNC1] | LCMS m/z [M + 1]: 633.0 ¹HNMR (400 MHz, CD₃OD) δ 8.26 (s, 1H), 7.27-7.15 (m, 1H), 7.00 (t, J = 9.2 Hz, 1H), 4.74- 4.58 (m, 3H), 4.33 (d, J = 6.4 Hz, 2H), 4.05- 3.87 (m, 2H), 3.51-3.38 (m, 4H), 3.11-2.90 (m, 2H), 2.63 (t, J = 2.8 Hz, 1H), 2.24-2.07 (m, 2H), 2.05-1.90 (m, 2H), 1.84-1.68 (m, 3H), 1.54- 1.40 (m, 2H). |
| 62a | 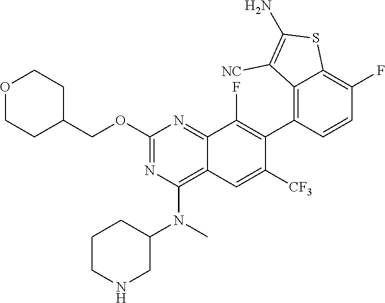 [SMILES: CN(c1nc(OCC2CCOCC2)nc2c(F)c(-c3ccc(F)c4sc(N)c(C#N)c34)c(C(F)(F)F)cc12)C1CCCNC1] | |
| 63 | 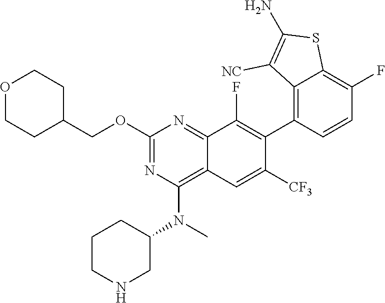 [SMILES: CN(c1nc(OCC2CCOCC2)nc2c(F)c(-c3ccc(F)c4sc(N)c(C#N)c34)c(C(F)(F)F)cc12)[C@H]1CCCNC1] | LCMS m/z [M + 1]: 633.2 ¹H NMR (400 MHz, CD₃OD) δ 8.26-8.13 (m, 1H), 8.14-7.96 (m, 2H), 7.27-7.19 (m, 1H), 7.17-7.08 (m, 1H), 4.51-4.39 (m, 1H), 4.38-4.16 (m, 2H), 3.92-3.82 (m, 2H), 3.30-3.22 (m, 4H), 3.18-3.04 (m, 2H), 3.94-2.74 (m, 2H), 2.48-2.34 (m, 2H), 2.10-1.72 (m, 4H), 1.70-1.61 (m, 2H), 1.57-1.45 (m, 1H), 1.38-1.26 (m, 2H). |

Table 3 includes selected compounds of the present disclosure.

TABLE 3
Selected compounds of the present disclosure

| Compound | | |
|---|---|---|
| No. | Structure | Analytical data |
| 64 | 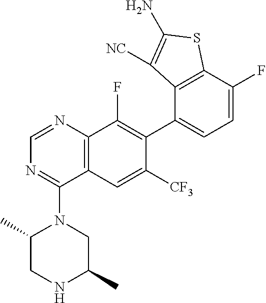 [SMILES: C[C@@H]1CN(c2ncnc3c(F)c(-c4ccc(F)c5sc(N)c(C#N)c45)c(C(F)(F)F)cc23)[C@@H](C)CN1] | LCMS m/z [M + 1]: 519 ¹H NMR (400 MHz, CD₃OD) δ 8.95 (d, 1H), 8.30 (d, 1H), 7.20-7.35 (m, 1H), 7.04 (t, 1H), 4.49-4.68 (m, 1H), 3.72-4.11 (m, 3H), 3.58- 3.69 (m, 1H), 3.33-3.45 (m, 1H), 1.50-1.68 (m, 3H), 1.27-1.46 (m, 3H). |
| 64a | 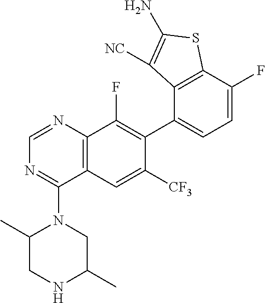 [SMILES: CC1CN(c2ncnc3c(F)c(-c4ccc(F)c5sc(N)c(C#N)c45)c(C(F)(F)F)cc23)C(C)CN1] | |
| 65 | 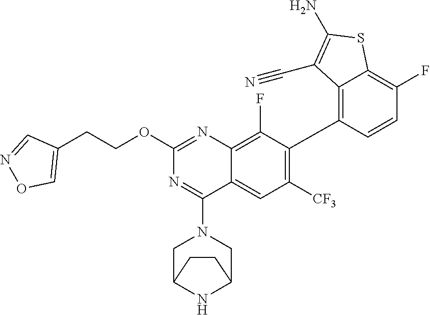 [SMILES: N#Cc1c(N)sc2c(F)ccc(-c3c(C(F)(F)F)cc4c(N5CC6CCC(C5)N6)nc(OCCc5cnoc5)nc4c3F)c12] | LCMS m/z [M + 1]: 628.0 ¹H NMR (400 MHz, CD₃OD) δ 8.41 (s, 1H), 8.27 (s, 1H), 8.18 (s, 1H), 7.25-7.19 (m, 1H), 7.05-6.98 (m, 1H), 4.65-4.59 (m, 1H), 4.55-4.47 (m, 1H), 3.81-3.62 (m, 6H), 2.72-2.61 (m, 1H), 2.52-2.45 (m, 1H), 1.89-1.73 (m, 4H). |
| 66 | 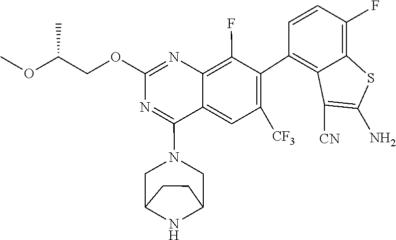 [SMILES: CO[C@H](C)COc1nc(N2CC3CCC(C2)N3)c2cc(C(F)(F)F)c(-c3ccc(F)c4sc(N)c(C#N)c34)c(F)c2n1] | LCMS m/z [M + 1]: 605.2 ¹H NMR (400 MHz, CD₃OD) δ 8.11 (s, 1H), 7.19-7.24 (m, 1H), 6.99-7.05 (m, 1H), 4.59-4.79 (m, 2H), 4.42-4.50 (m, 2H), 4.19-4.25 (m, 2H), 3.75-3.95 (m, 3H), 3.42 (s, 3H), 2.06-2.18 (m, 4H), 1.24-1.30 (m, 3H). |
| 66a | 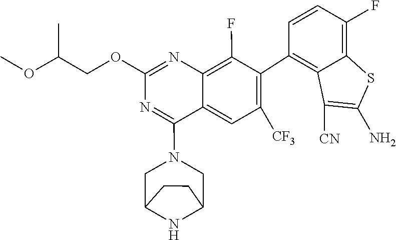 [SMILES: COC(C)COc1nc(N2CC3CCC(C2)N3)c2cc(C(F)(F)F)c(-c3ccc(F)c4sc(N)c(C#N)c34)c(F)c2n1] | |
| 67 | 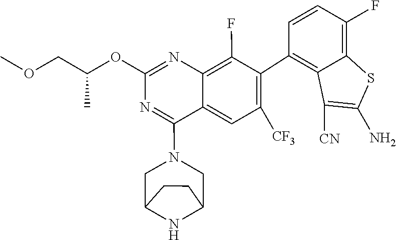 [SMILES: COC[C@@H](C)Oc1nc(N2CC3CCC(C2)N3)c2cc(C(F)(F)F)c(-c3ccc(F)c4sc(N)c(C#N)c34)c(F)c2n1] | LCMS m/z [M + 1]: 605.3 ¹H NMR (400 MHz, CD₃OD) δ 8.13 (s, 1H), 7.22 (dd, J = 8.3, 5.0 Hz, 1H), 7.00-7.06 (m, 1H), 5.52-5.60 (m, 1H), 4.59-4.82 (m, 2H), 4.20-4.30 (m, 2H), 3.85-4.02 (m, 2H), 3.56-3.69 (m, 2H), 3.39 (s, 3H), 2.04-2.22 (m, 4H), 1.40 (d, J = 6.4 Hz, 3H). |
| 67a | 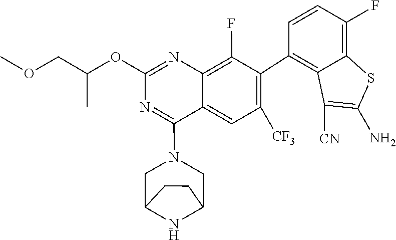 [SMILES: COCC(C)Oc1nc(N2CC3CCC(C2)N3)c2cc(C(F)(F)F)c(-c3ccc(F)c4sc(N)c(C#N)c34)c(F)c2n1] | |
| 68 | 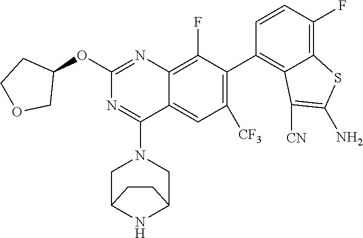 [SMILES: N#Cc1c(N)sc2c(F)ccc(-c3c(C(F)(F)F)cc4c(N5CC6CCC(C5)N6)nc(O[C@@H]5CCOC5)nc4c3F)c12] | LCMS m/z [M + 1]: 603.2 ¹H NMR (400 MHz, CD₃OD) δ 8.12 (s, 1H), 7.19-7.25 (m, 1H), 7.02 (t, J = 8.8 Hz, 1H), 5.70- 5.74 (m, 1H), 4.73-4.77 (m, 1H), 4.61-4.65 (m, 1H), 4.20-4.28 (m, 2H), 3.86-4.10 (m, 6 H), 2.12-2.38 (m, 6H). |
| 68a | 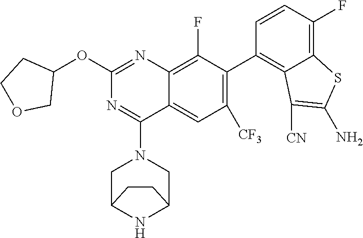 [SMILES: N#Cc1c(N)sc2c(F)ccc(-c3c(C(F)(F)F)cc4c(N5CC6CCC(C5)N6)nc(OC5CCOC5)nc4c3F)c12] | |
| 69 | 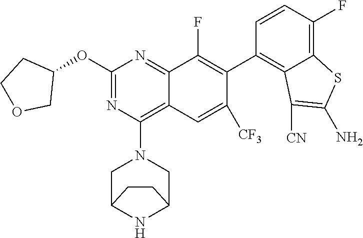 [SMILES: N#Cc1c(N)sc2c(F)ccc(-c3c(C(F)(F)F)cc4c(N5CC6CCC(C5)N6)nc(O[C@H]5CCOC5)nc4c3F)c12] | LCMS m/z [M + 1]: 603.2 ¹H NMR (400 MHz, CD₃OD) δ 8.12 (s, 1H), 7.21 (dd, J = 5.2 and 8.4 Hz), 7.02 (t, J = 8.8 Hz, 1H), 5.72 (d, J = 5.2 Hz, 1H), 4.74 (d, J = 3.6 Hz, 1H), 4.62 (d, J = 13.6 Hz, 1H), 4.24 (s, 2H), 4.10- 4.06 (m, 1H), 4.03-3.97 (m, 2H), 3.93-3.88 (m, 2H), 3.84 (d, J = 14.0 Hz, 1H), 2.40-2.31 (m, 1H), 2.24-2.20 (m, 1H), 2.13 (s, 4H). |
| 70 | 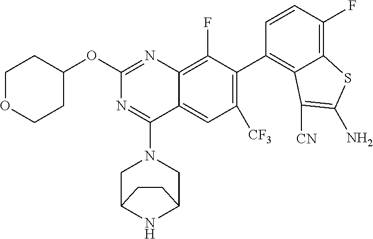 [SMILES: N#Cc1c(N)sc2c(F)ccc(-c3c(C(F)(F)F)cc4c(N5CC6CCC(C5)N6)nc(OC5CCOCC5)nc4c3F)c12] | LCMS m/z [M + 1]: 617.3 ¹H NMR (400 MHz, CD₃OD) δ 8.12 (s, 1H), 7.19-7.23 (m, 1H), 7.02 (t, J = 9.2 Hz, 1H), 5.37- 5.44 (m, 1H), 4.71-4.75 (m, 1H), 4.60-4.64 (m, 1H), 4.20-4.28 (m, 2H), 3.84-4.00 (m, 4 H), 3.60-3.68 (m, 2H), 1.95-2.20 (m, 6H), 1.81-1.89 (m, 2H). |
| 71 | 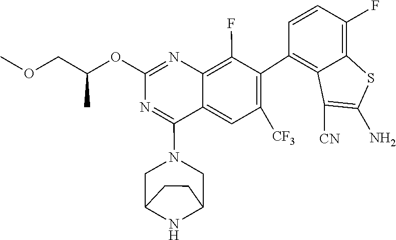 [SMILES: COC[C@H](C)Oc1nc(N2CC3CCC(C2)N3)c2cc(C(F)(F)F)c(-c3ccc(F)c4sc(N)c(C#N)c34)c(F)c2n1] | LCMS m/z [M + 1]: 605.3 ¹H NMR (400 MHz, CD₃OD) δ 8.13 (s, 1H), 7.19-7.24 (m, 1H), 7.02 (t, J = 9.2 Hz, 1H), 5.52- 5.59 (m, 1H), 4.59-4.79 (m, 2H), 4.20-4.30 (m, 2H) 3.78-4.00 (m, 2H), 3.57-3.67 (m, 2H), 3.38 (s, 3H), 2.03-2.20 (m, 4H), 1.39 (d, J = 6.4 Hz, 3H) |
| 72 | 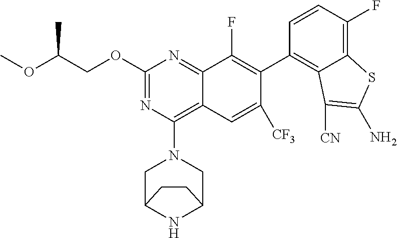 [SMILES: CO[C@@H](C)COc1nc(N2CC3CCC(C2)N3)c2cc(C(F)(F)F)c(-c3ccc(F)c4sc(N)c(C#N)c34)c(F)c2n1] | LCMS m/z [M + 1]: 605.2 ¹H NMR (400 MHz, CD₃OD) δ 8.42 (s, 1H), 7.23 (dd, J = 5.2 and 8.4 Hz, 1H), 7.03 (t, J = 8.8 Hz, 1H), 6.86 (d, J = 49.2 Hz, 1H), 5.56 (d, J = 52.0 Hz, 1H), 5.04-5.01 (m, 1H), 4.83- 4.78 (m, 1H), 4.69 (s, 2H), 4.01-3.88 (m, 4H), 3.66 (s, 3H), 3.51-3.45 (m, 1H), 2.74-2.32 (m, 8H), 2.19-2.13 (m, 1H), 1.13 (J = 6.8 Hz, 3H). |
| 73 | 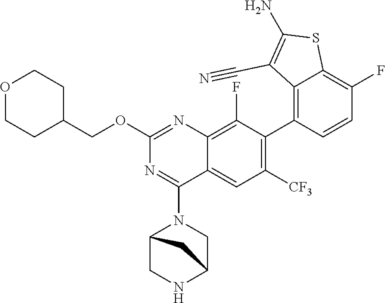 [SMILES: N#Cc1c(N)sc2c(F)ccc(-c3c(C(F)(F)F)cc4c(N5C[C@H]6C[C@@H]5CN6)nc(OCC5CCOCC5)nc4c3F)c12] | |
| 73a | 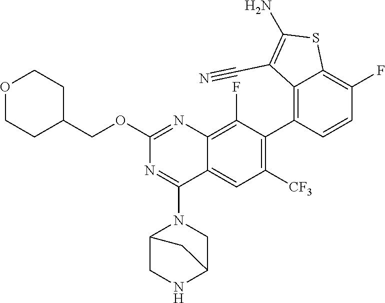 [SMILES: N#Cc1c(N)sc2c(F)ccc(-c3c(C(F)(F)F)cc4c(N5CC6CC5CN6)nc(OCC5CCOCC5)nc4c3F)c12] | |
| 74 | 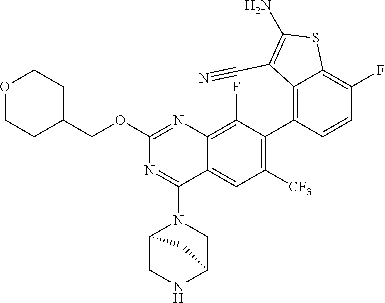 [SMILES: N#Cc1c(N)sc2c(F)ccc(-c3c(C(F)(F)F)cc4c(N5C[C@@H]6C[C@H]5CN6)nc(OCC5CCOCC5)nc4c3F)c12] | |
| 75 | 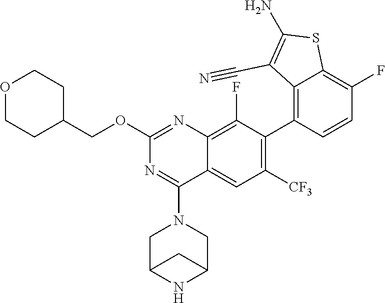 [SMILES: N#Cc1c(N)sc2c(F)ccc(-c3c(C(F)(F)F)cc4c(N5CC6CC(C5)N6)nc(OCC5CCOCC5)nc4c3F)c12] | |
| 76 | 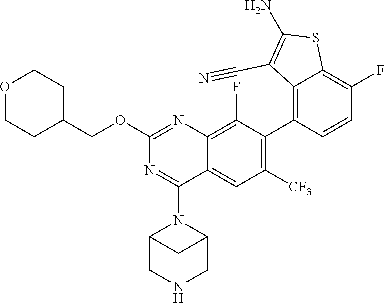 [SMILES: N#Cc1c(N)sc2c(F)ccc(-c3c(C(F)(F)F)cc4c(N5C6CNCC5C6)nc(OCC5CCOCC5)nc4c3F)c12] | |
| 77 | 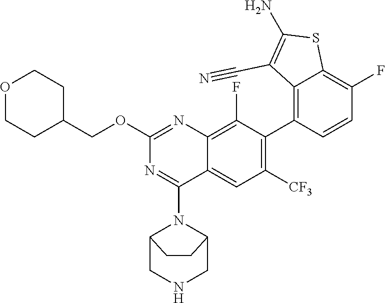 [SMILES: N#Cc1c(N)sc2c(F)ccc(-c3c(C(F)(F)F)cc4c(N5C6CCC5CNC6)nc(OCC5CCOCC5)nc4c3F)c12] | |
| 78 | 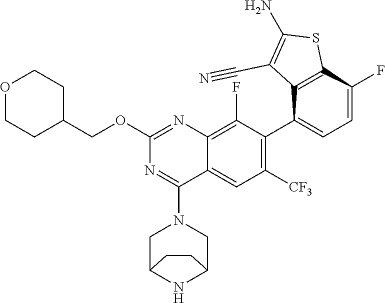 | |
| 79 | 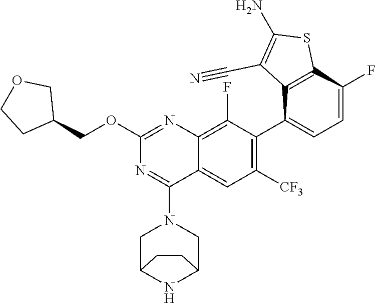 | |
| 80 | 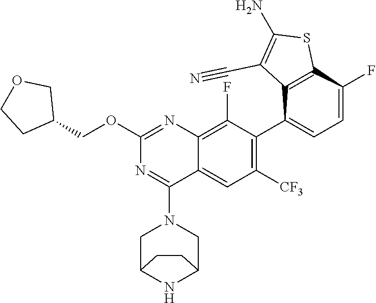 | |

### Biological Example 1: Disrupting RAS-Effector Binding HTRF Assay

A protein:protein interaction (PPI) Homogeneous Time Resolved Fluorescence (HTRF) assay was used to determine the effectiveness of compounds of the present disclosure in disrupting KRAS protein and effector RAF1 binding.

The HTRF assay used the following reagents and proteins: (i) 50 nM Avi-KRAS G12D (1-169) GppNHp/RAF1 RBD-3×FLAG (52-151); (ii) 50 nM Avi-KRAS G12R (1-169) GppNHp/RAF1 RBD-3×FLAG (52-151); (iii) 50 nM Avi-KRAS G12V (1-169) GppNHp/RAF1 RBD-3×FLAG (52-151), (iv) 50 nM Avi-KRAS WT (1-169) GppNHp/RAF1 RBD-3×FLAG (52-151); and (v) 75 nM Avi-RAF1 RBD-3×FLAG. The assay buffer included: 50 mM Tris pH 7.5, 100 mM NaCl, 5 mM MgCl₂, 0.1% BSA, 0.0100 Tween, 10% DMSO. The bead buffer included: 50 mM Tris pH 7.5, 0.01% o Tween 20. Assay volume of 20 μL was used in a 384 well plate-low volume format. Compound titration: 30-0.0045 μM, 3× dilution series.

The HTRF assay employed the following protocol:

Compounds were dispensed in assay plate (384-well, Grenier Bione #784075) using Echo (model 555) with dose response settings: 200 nL final volume, titration from 30 μM as a 10-point dilution series. Protein mix was prepared in assay buffer, and dispensed on plates, 10 μL per well, then incubated for 1 h at room temperature, with 700 rpm shaking. Reagents mix was prepared and dispensed on plates, 10 μL per well, then incubated for 1 h at room temperature, with 700 rpm shaking.

Plates were analyzed on an Envision plate reader using the following setting: Excitation 320 nM, Bandwidth 75 nM; Emission 615 nM, Bandwidth 85 nM, Gain 100%, Flashes 100, Lag 60 μs. Data were reported as percentage of activity, with DMSO as 100%. Data were plotted and analyzed using Prism 8.

The biochemical Raf1-KRAS G12D/G12R/G12V/WT-GppNHP disruption assay IC₅₀ of selected compounds described herein are shown in Table 4.

### Biological Example 2: Cell-Based pERK HTRF Assay in GP2d Cell

A pERK assay (Perkin Elmer) was used to determine the effectiveness of compounds in disrupting KRAS G12D protein/effector signaling in cells.

On Day 1, cells (GP2d) were seeded into 96-well plates at 3×10⁴ cells/well in 80 μL complete growth media (DMEM, 10% FBS).

On Day 2, cells were treated with compounds at 0.25% DMSO. The source plate was created with compounds diluted in media at 5-fold the final assay concentration. The compounds were run in a 9-point concentration curve starting at 1 M, with a half-log dilution between concentrations. 20 μL was transferred onto the cell plates (final volume in wells was 100 μL). Plates were harvested after 60 min incubation by aspirating media and adding kit-supplied 1× supplemented lysis buffer to all wells (50 μL per well). Plates were then placed on a plate shaker and incubated at 850 rpm for an additional 30 min.

Antibody mixture solution was prepared by diluting aliquoted d2 and Eu Cryptate antibodies 1:20 in kit supplied detection buffer, and mixing the diluted antibodies solutions (1:1 v:v). 4 μL of this solution was then added to a 384-well detection plate (Perkin Elmer; 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. Maximum signal was reached after 4 h incubation time and remained stable over a period of 24 hours. Therefore, readings could be made between 4 and 24 h of incubation. Plates were centrifuged again (524 g for 1 min), then analyzed on the EnVision plate reader using the following settings: Excitation 320 nm, Bandwidth 75 nm; Emission 615 nm, Bandwidth 85 nm; Gain 100%; Flashes 100; Lag 60 μs.

### Biological Example 3: Cell-Based pERK HTRF Assay in SW620 Cell

pERK assay (Perkin Elmer) was used to determine the effectiveness of compounds in disrupting KRAS G12V protein/effector signaling in cells.

On Day 1, cells (SW620) were seeded into 96-well plates at 3×10⁴ cells/well in 80 μL complete growth media (DMEM, 10% FBS).

On Day 2, cells were treated with compounds at 0.25% DMSO. The source plate was created with compounds diluted in media at 5-fold the final assay concentration. The compounds were run in a 9-point concentration curve starting at 10 μM, with a half-log dilution between concentrations. 20 μL was transferred onto the cell plates (final volume in wells was 100 μL). Plates were harvested after 1 h incubation by aspirating media and adding kit-supplied 1× supplemented lysis buffer to all wells (50 μL per well). Plates were then placed on a plate shaker and incubated at 850 rpm for an additional 30 min.

Antibody mixture solution was prepared by diluting aliquoted d2 and Eu Cryptate antibodies 1:20 in kit supplied detection buffer, and mixing the diluted antibodies solutions (1:1 v:v). Four μL of this solution was then added to a 384-well detection plate (Perkin Elmer; 6008230).

Samples were homogenized by pipetting up and down and then transferred (16 μL of cell lysates) from the 96-well cell culture plate to two wells of the HTRF 384-well detection plate containing the antibody solution. Plates were centrifuged (524 g for 1 min) and allowed to incubate between 4 and 24 h at room temperature. Maximum signal was reached after 4 h incubation time and remained stable over a period of 24 hours. Therefore, readings could be made between 4 and 24 h of incubation. Plates were centrifuged again (524 g for 1 min), then analyzed on the EnVision plate reader using the following settings: Excitation 320 nm, Bandwidth 75 nm; Emission 615 nm, Bandwidth 85 nm; Gain 100%; Flashes 100; Lag 60 μs.

For Raf1 RBD-KRAS G12D-GppNHp, Raf1 RBD-KRAS G12V-GppNHp, Raf1 RBD-KRAS G12R-GppNHp, and Raf1 RBD-KRAS WT-GppNHp disruption assays: A: IC₅₀≤0.1 μM; B: 0.1 μM<IC₅₀≤1 μM; C: 1 μM<IC₅₀≤10 μM; D: IC₅₀>10 M. For Gp2d pERK HTRF assay: A: IC₅₀≤0.1 μM; B: 0.1 μM<IC₅₀≤1 μM; C: IC₅₀>1 μM. For SW620 pERK HTRF assay: A: IC₅₀≤0.1 μM; B: 0.1 μM<IC₅₀≤1 μM; C: 1 μM<IC₅₀≤10 μM; D: IC₅₀>10 μM. Blanks in the table represent that a compound was not tested in the indicated assay.

TABLE 4
Biological characterization of selected compounds of the present disclosure.

| | Raf1 RBD- | Raf1 RBD- | Raf1 RBD- | Raf1 RBD- | | |
|---|---|---|---|---|---|---|
| | KRAS G12D- | KRAS G12V- | KRAS G12R- | KRAS WT- | | |
| Compound | GppNHp | GppNHp | GppNHp | GppNHp | pERK- | pERK- |
| No. | disruption | disruption | disruption | disruption | Gp2D | SW620 |
| 1 | C | D | D | C | A | |
| 2 | D | D | D | D | D | |
| 3 | C | D | D | C | B | |
| 4 | C | D | D | C | B | |
| 5 | C | D | D | C | A | |
| 6 | C | D | D | C | B | |
| 7 | C | D | D | D | B | |
| 8 | C | D | D | C | B | |
| 9 | C | D | D | C | B | |
| 10 | C | D | D | D | C | |
| 11 | C | D | D | D | B | |
| 12 | C | D | D | C | B | |
| 13 | C | D | D | D | B | |
| 14 | C | D | D | C | B | |
| 15 | C | D | D | C | A | |
| 16 | C | D | D | C | B | |
| 17 | C | D | D | C | B | |
| 18 | B | C | C | C | A | |
| 19 | C | D | D | C | A | |
| 20 | C | D | D | C | B | |
| 21 | D | D | D | C | B | |
| 22 | C | D | D | C | A | |
| 23 | C | D | D | C | A | |
| 24 | B | C | C | B | A | |
| 25 | C | D | D | C | B | |
| 26 | C | D | D | C | B | |
| 27 | D | D | D | C | B | |
| 28 | C | D | D | D | A | D |
| 29 | B | C | C | C | A | C |
| 30 | B | C | C | B | A | D |
| 31 | B | D | D | C | A | D |
| 32 | B | D | D | C | A | D |
| 33 | B | D | D | C | A | D |
| 34 | B | C | D | C | B | D |
| 35 | D | D | D | D | B | D |
| 36 | B | D | D | C | A | D |
| 37 | B | D | D | C | A | D |
| 38 | C | D | D | C | A | D |
| 39 | C | D | D | C | B | |
| 40 | B | C | C | B | A | D |
| 41 | B | D | D | C | A | D |
| 42 | B | D | C | C | A | D |
| 43 | D | D | D | D | C | D |
| 44 | C | D | D | C | A | D |
| 45 | B | C | C | C | A | D |
| 46 | B | D | D | C | A | D |
| 47 | B | D | D | C | A | D |
| 48 | C | D | D | C | A | D |
| 49 | C | D | D | C | A | D |
| 50 | B | D | D | C | A | D |
| 51 | B | D | D | C | A | D |
| 52 | C | D | D | C | A | D |
| 53 | B | C | D | B | A | D |
| 54 | B | D | D | C | A | D |
| 55 | B | C | C | B | A | D |
| 56 | B | C | C | B | A | D |
| 57 | B | D | D | C | A | D |
| 58 | C | D | D | D | B | |
| 59 | C | D | D | D | B | |
| 60 | D | D | D | D | C | |
| 61 | C | D | D | D | C | D |
| 62 | C | D | D | D | C | D |
| 63 | D | D | D | D | C | D |
| 64 | C | D | D | D | A | |
| 65 | B | D | D | C | A | D |
| 66 | | | | | A | D |
| 67 | | | | | A | D |
| 68 | | | | | A | D |
| 69 | | | | | A | D |
| 70 | | | | | A | D |
| 71 | | | | | A | D |
| 72 | | | | | A | D |

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations, and equivalents. It is intended that the following claims define the scope of the invention and that methods and structures within the scope of these claims and their equivalents be covered thereby.

1. A compound represented by Formula 1: 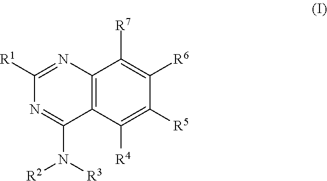
or a salt (e.g., pharmaceutically acceptable salt) thereof, wherein:
R¹ is selected from H, C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷;
R² is selected from H and C₁₋₆alkyl, wherein any C₁₋₆ alkyl is unsubstituted or is substituted with one or more R¹³;
R³ is selected from a 4-6 membered heterocycle that is unsubstituted or substituted with one or more R¹⁰;
or R² and R³, together with the nitrogen atom to which they are attached, form a 4-9 membered heterocycle that is unsubstituted or is substituted with one or more R¹¹;
R⁴ is selected from H and —OR¹⁴;
R⁵ is selected from H, halogen, —CN, —OR¹², a 3-6 membered heterocycle, a 5-6 membered heteroaryl, and C₁₋₆alkyl, wherein any C₁₋₆alkyl is unsubstituted or substituted with one or more R¹³;
R⁶ is a bicyclic heteroaryl substituted with one or more R¹⁵;
R⁷ is selected from halogen;
R⁸ is selected from H, C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R⁹;
each R¹⁰ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹¹ is independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰;
each R¹² is independently selected from C₁₋₆ alkyl, C₂₋₆ alkenyl, and H, wherein any C₁₋₆alkyl or C₂₋₆ alkenyl is unsubstituted or substituted with one or more R¹³;
each R¹³ is independently selected from —OR¹⁴, —CN, —N(R¹⁴)₂, and halogen;
each R¹⁴ is independently selected from C₁₋₆ alkyl, C₂₋₆ alkenyl, and H;
each R¹⁵ is independently selected from halogen, —N(R¹²)₂, —CN, and C₁₋₆alkyl, wherein any C₁₋₆alkyl is unsubstituted or substituted with one or more R¹³;
each R¹⁶ is independently selected from H, C₁₋₆alkyl, —C(O)C₁₋₆alkyl, and —C(O)(C₀₋₆alkylene)heteroaryl, wherein any C₁₋₆alkyl and C₁₋₆alkylene are unsubstituted or substituted with one or more R¹³, and wherein any heteroaryl is unsubstituted or substituted with one or more R¹⁸;
each R¹⁷ is independently selected from —OR¹², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
each R¹⁸ is independently selected from C₁₋₆alkyl;
each R¹⁹ is independently selected from —OR², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl, wherein any C₁₋₆alkyl is unsubstituted or substituted with one or more R²⁰; and
each R²⁰ is independently selected from —OH, —OC₁₋₆alkyl, —CN, —NH₂, —NHC₁₋₆alkyl, and halogen.

2. The compound of claim 1, wherein:
R¹ is selected from C₁₋₆alkyl, —OR⁸, —O(C₁₋₆alkylene)R⁸, —N(R¹⁶)₂, and a 4-7 membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷,
provided that R¹ is not: 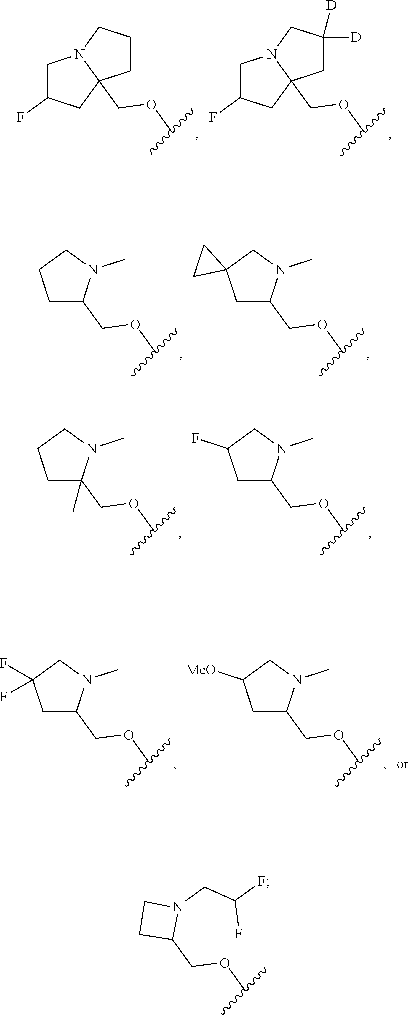
and
R⁶ is

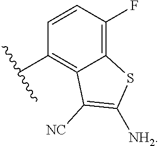 [SMILES: *c1ccc(F)c2sc(N)c(C#N)c12]

3. The compound of claim 1, wherein:
R¹ is selected from C₁₋₆alkyl, —OR⁸, —O(C₁₋₆alkylene)R⁸, —N(R¹⁶)₂, and a 4-, 5- or 7-membered nitrogen-containing heterocycle, wherein any C₁₋₆alkyl and C₁₋₆alkylene are unsubstituted or substituted with one or more R¹³, and wherein the nitrogen containing-heterocycle is unsubstituted or substituted with one or more R¹⁷; and
R⁸ is selected from C₁₋₆alkyl, carbocycle, aryl, heteroaryl, and heterocycle, wherein any C₁₋₆alkyl is unsubstituted or substituted with one or more R¹³, wherein any carbocycle, aryl, heteroaryl, or heterocycle comprises 3-8 members and is unsubstituted or substituted with one or more R¹⁹,
provided that R⁸ is not an unsubstituted or substituted heterocycle selected from:

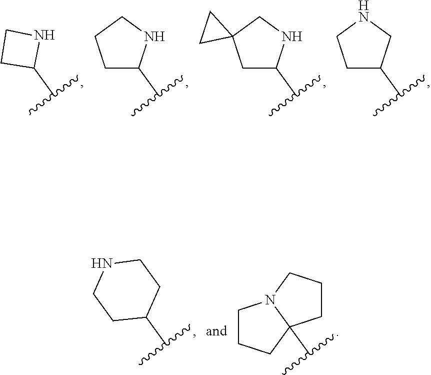 [SMILES: *C1CCN1 ; *C1CCCN1 ; *C1CC2(CC2)CN1 ; *C1CCNC1 ; *C1CCNCC1 ; *C12CCCN1CCC2]

4. The compound of claim 1 or 3, wherein R⁶ is selected from: 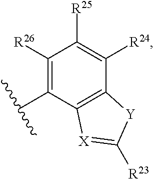
wherein:
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

5. The compound of claim 4, wherein X is C—CN and Y is S.

6. The compound of claim 4 or 5, wherein R²³ is —N(R¹²)₂.

7. The compound of any one of claims 4-6, wherein X is C—CN, Y is S, and R²³ is —NH₂.

8. The compound of any one of claims 4-7, wherein X is C—CN, Y is S, R²³ is —N(R¹²)₂, and R²⁴ is a halogen (e.g., F).

9. The compound of any one of claims 1 or 3-8, wherein R⁶ is selected from: 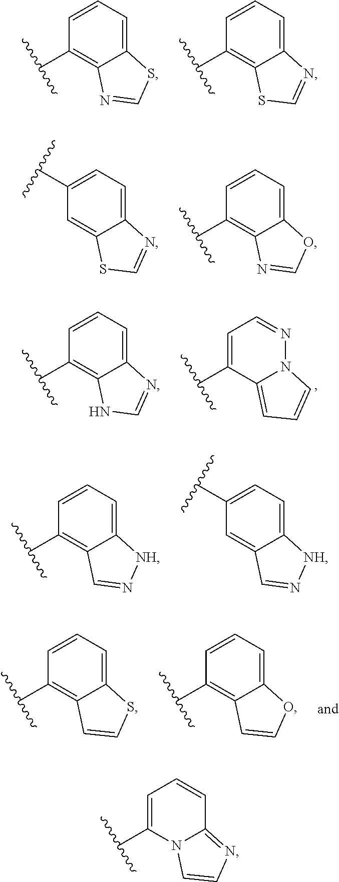
any of which is substituted with one or more R¹⁵.

10. The compound of any one of claims 1 or 3-9, wherein R⁶ is selected from:

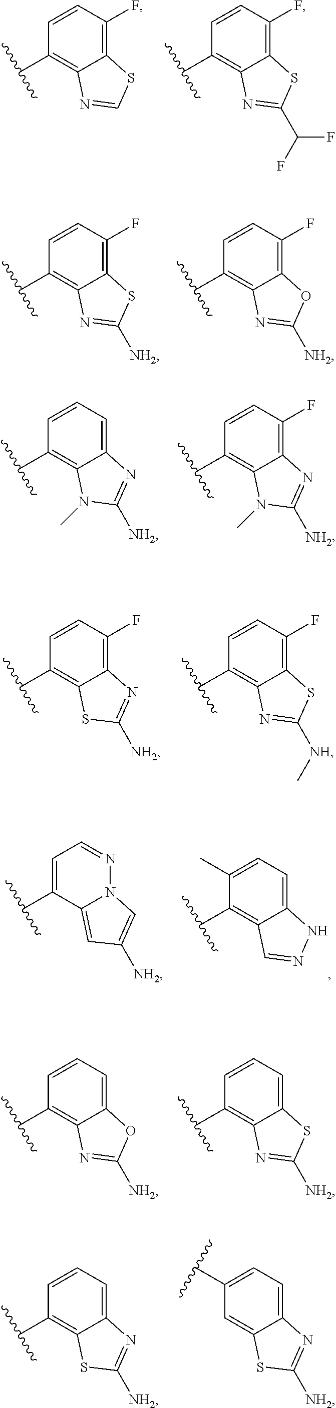

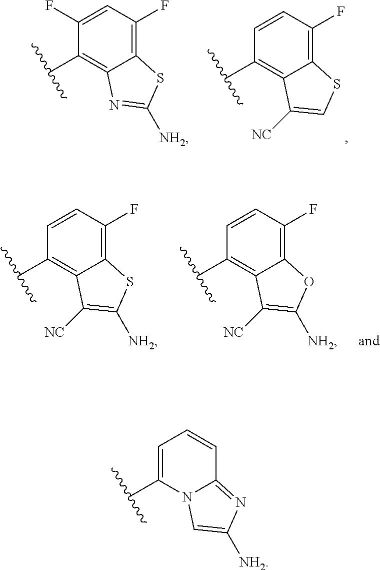 [SMILES: *c1c(F)cc(F)c2sc(N)nc12 ; *c1ccc(F)c2scc(C#N)c12 ; *c1ccc(F)c2sc(N)c(C#N)c12 ; *c1ccc(F)c2oc(N)c(C#N)c12 ; *c1cccc2nc(N)cn12]

11. The compound of any one of claims 1 or 3-10, wherein R⁶ is selected from:

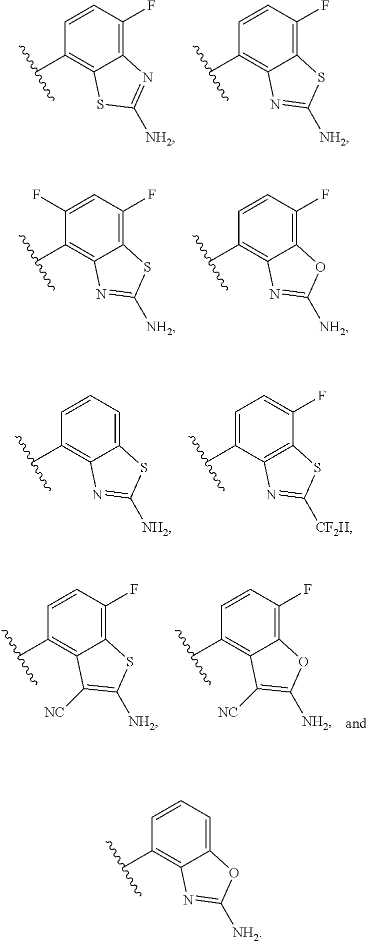

12. The compound of claim 11, wherein R⁶ is

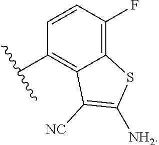 [SMILES: *c1ccc(F)c2sc(N)c(C#N)c12]

13. The compound of any one of claims 1-12, wherein R² is selected from H and C₁₋₆alkyl, wherein any C₁₋₆ alkyl is unsubstituted or is substituted with one or more R¹³; and R³ is selected from a 4-6 membered heterocycle that is unsubstituted or substituted with one or more R¹⁰.

14. The compound of claim 13, wherein R³ is a 4-6 membered heterocycle that includes one or more heteroatoms selected from O, S, and N.

15. The compound of claim 14, wherein R³ is a 4-6 membered heterocycle that includes 1 heteroatom selected from O, S, and N.

16. The compound of claim 14 or 15, wherein R³ is a 4-6 membered heterocycle that includes a nitrogen atom.

17. The compound of claim 16, wherein R³ is an azetidine.

18. The compound of claim 16, wherein R³ is a pyrrolidine.

19. The compound of claim 16, wherein R³ is a piperidine.

20. The compound of any one of claims 16-19, wherein R³ includes an —NH— moiety.

21. The compound of any one of claims 13-20, wherein at least one R¹⁰ is selected from —OR¹² and a C₁₋₆alkyl substituted with —OH.

22. The compound of any one of claims 13-21, wherein at least one R¹⁰ is an unsubstituted C₁₋₆alkyl.

23. The compound of any one of claims 13-20, wherein R³ is unsubstituted.

24. The compound of any one of claims 1-23, wherein the compound is a compound according to Formula IB, IC, ID, or IE: 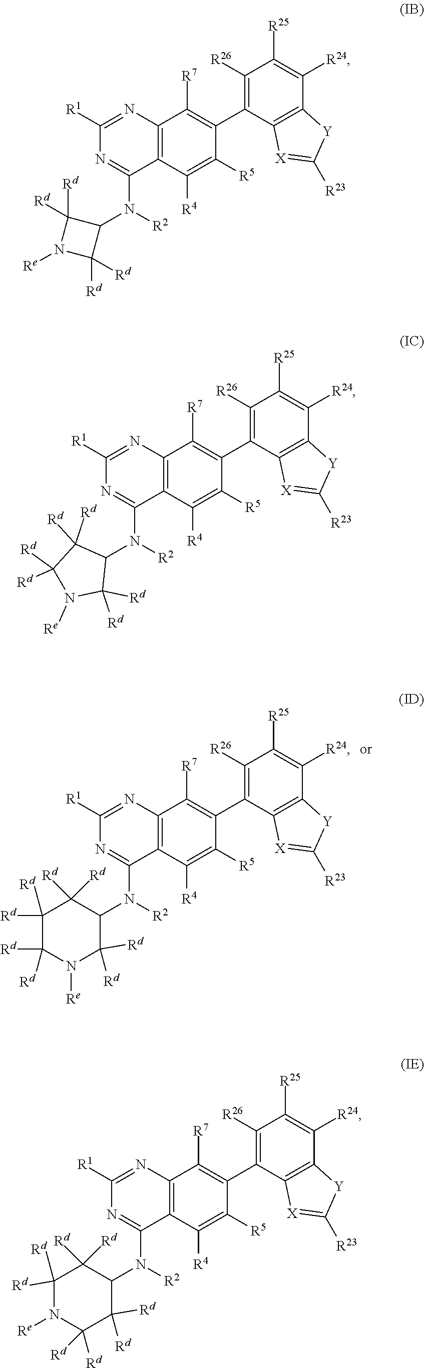
or a salt (e.g., pharmaceutically acceptable salt) thereof, wherein:
each R^{d} is independently selected from H, —OR², ═O, —C(O)(C₁₋₆alkylene)CN, —C(O)(C₁₋₆alkylene)OH, —C(O)(C₁₋₆alkyl), —S(O)₂(C₁₋₆alkyl), halogen, and C₁₋₆alkyl, wherein any C₁₋₆alkyl is unsubstituted or substituted with one or more R²⁰;
R^{e} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

25. The compound of claim 24, wherein the compound is a compound according to Formula IB, or a salt (e.g., pharmaceutically acceptable salt) thereof.

26. The compound of claim 24, wherein the compound is a compound according to Formula IC, or a salt (e.g., pharmaceutically acceptable salt) thereof.

27. The compound of claim 24, wherein the compound is a compound according to Formula ID, or a salt (e.g., pharmaceutically acceptable salt) thereof.

28. The compound of claim 24, wherein the compound is a compound according to Formula IE, or a salt (e.g., pharmaceutically acceptable salt) thereof.

29. The compound of any one of claims 24-28, wherein X is C—CN and Y is S.

30. The compound of any one of claims 24-29, wherein R²³ is —N(R¹²)₂.

31. The compound of any one of claims 24-30, wherein X is C—CN, Y is S, R²³ is —N(R¹²)₂, and R²⁴ is a halogen (e.g., F).

32. The compound of any one of claims 24-31, wherein 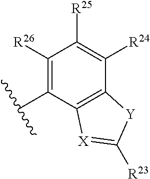
is

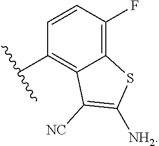 [SMILES: *c1ccc(F)c2sc(N)c(C#N)c12]

33. The compound of any one of claims 25-32, wherein each R^{d} is H.

34. The compound of any one of claims 25-32, wherein at least one R^{d} is selected from —OR¹² and a C₁₋₆alkyl substituted with —OH.

35. The compound of any one of claims 25-32, wherein 0-2 R^{d} groups are C₁₋₆ alkyl, and the remaining R^{d} groups are H.

36. The compound of any one of claims 25-35, wherein R^{e} is H.

37. The compound of any one of claims 25-35, wherein R^{e} is C₁₋₆alkyl that is unsubstituted or substituted with one or more R²⁰.

38. The compound of claim 37, wherein R^{e} is C₁₋₆alkyl that is unsubstituted.

39. The compound of claim 37, wherein R^{e} is C₁₋₆alkyl that is substituted with one or more R²⁰, wherein each R²⁰ is independently selected from —OH, —OC₁₋₆alkyl, ═O, and —CN.

40. The compound of any one of claims 13-39, wherein R² is H.

41. The compound of any one of claims 13-39, wherein R² is a C₁₋₆alkyl that is unsubstituted or is substituted with one or more R¹³.

42. The compound of claim 41, wherein R² is a C₁₋₂alkyl that is unsubstituted or is substituted with one or more R¹³.

43. The compound of claim 41 or 42, wherein R² is unsubstituted.

44. The compound of claim 41 or 42, wherein R² is substituted with one or more R¹³.

45. The compound of any one of claims 1-12, wherein R² and R³, together with the nitrogen atom to which they are attached, form a 4-9 membered heterocycle that is unsubstituted or is substituted with one or more R¹¹.

46. The compound of claim 45, wherein R² and R³, together with the nitrogen atom to which they are attached, form a 4-6 membered heterocycle that is unsubstituted or is substituted with one or more R¹¹.

47. The compound of claim 46, wherein the 4-6 membered heterocycle includes 1 or 2 heteroatoms selected from O, S, and N.

48. The compound of claim 47, wherein the 4-6 membered heterocycle includes 2 nitrogen atoms.

49. The compound of claim 48, wherein the 4-6 membered heterocycle is a piperazine.

50. The compound of any one of claims 46-49, wherein the 4-6 membered heterocycle includes an —NH— moiety.

51. The compound of claim 46 or 47, wherein the 4-6 membered heterocycle is an azetidine.

52. The compound of claim 46 or 47, wherein the 4-6 membered heterocycle is a pyrrolidine.

53. The compound of claim 46 or 47, wherein the 4-6 membered heterocycle is a piperidine.

54. The compound of any one of claims 46-53, wherein the 4-6 membered heterocycle is substituted with one or more R¹¹.

55. The compound of any one of claims 46-54, wherein at least one R¹¹ is selected from —OR¹² and a C₁₋₆alkyl substituted with —OH.

56. The compound of any one of claims 46-55, wherein at least one R¹¹ is an unsubstituted C₁₋₆alkyl.

57. The compound of any one of claims 46-53, wherein the 4-6 membered heterocycle is unsubstituted.

58. The compound of claim 45, wherein R² and R³, together with the nitrogen atom to which they are attached, form a bridged heterocycle that is unsubstituted or is substituted with one or more R¹¹.

59. The compound of claim 58, wherein R² and R³, together with the nitrogen atom to which they are attached, form a 7-9 membered bridged heterocycle that includes 1 or 2 heteroatoms selected from O, S, and N.

60. The compound of claim 59, wherein the bridged heterocycle includes 2 nitrogen atoms.

61. The compound of claim 60, wherein the bridged heterocycle is a bridged piperazine.

62. The compound of claim 60 or 61, wherein the bridged heterocycle includes an —NH— moiety.

63. The compound of claim 59, wherein the bridged heterocycle is a bridged piperidine.

64. The compound of any one of claims 58-63, wherein the bridged heterocycle is substituted with one or more R¹¹, wherein at least one R¹¹ is selected from —OR¹² and a C₁₋₆alkyl substituted with —OH.

65. The compound of any one of claims 58-64, wherein the bridged heterocycle is substituted with one or more R¹¹, wherein at least one R¹¹ is an unsubstituted C₁₋₆alkyl.

66. The compound of any one of claims 58-63, wherein the bridged heterocycle is unsubstituted.

67. The compound of claim 45, wherein R² and R³, together with the nitrogen atom to which they are attached, form a spirocycle that is unsubstituted or is substituted with one or more R¹¹.

68. The compound of claim 67, wherein the spirocycle includes an —NH— moiety.

69. The compound of any one of claims 45-68, wherein R² and R³, together with the nitrogen atom to which they are attached, form a 4-9 membered heterocycle that is unsubstituted or is substituted with one or more R¹¹, wherein the heterocycle includes an —NH— moiety, and wherein the one or more R¹¹ are independently selected from —OR¹², ═O, —C(O)(C₁₋₆alkylene)CN, —C(O)(C₁₋₆alkylene)OR¹⁴, —C(O)(C₁₋₆alkyl), —S(O)₂(C₁₋₆alkyl), —N(R¹⁴)C(O)(C₁₋₆alkyl), —N(R¹⁴)C(O)(C₁₋₆alkylene)OR¹⁴, halogen, and C₁₋₆alkyl that is unsubstituted or substituted with one or more R²⁰.

70. The compound of any one of claims 1-12 and 45-69, wherein the compound is a compound according to Formula IF: 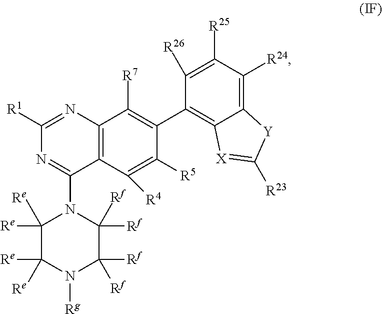
or a salt (e.g., pharmaceutically acceptable salt) thereof, wherein:
each R^{e} and R^{f} is independently selected from R¹¹ and hydrogen, wherein an R^{e} and an R^{f} can optionally join together to form a 4-6 membered ring, or a first R^{f} and a second R^{f} connected to adjacent atoms can optionally join together to form a 3-5 membered ring, or a first R^{e} and a second R^{e} connected to adjacent atoms can optionally join together to form a 3-5 membered ring;
R⁹ is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

71. The compound of claim 70, wherein X is C—CN and Y is S.

72. The compound of claim 70 or 71, wherein R²³ is —N(R¹²)₂.

73. The compound of any one of claims 70-72, wherein X is C—CN, Y is S, R²³ is —N(R¹²)₂, and R²⁴ is a halogen (e.g., F).

74. The compound of any one of claims 70-74, wherein 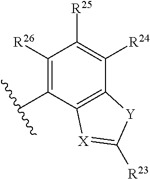
is

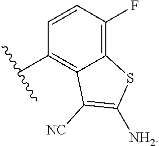 [SMILES: *c1ccc(F)c2sc(N)c(C#N)c12]

75. The compound of any one of claims 71-74, wherein R^{g} is selected from H and C₁₋₆alkyl that is unsubstituted.

76. The compound of claim 75, wherein R^{g} is H.

77. The compound of claim 75, wherein R^{g} is C₁₋₆alkyl that is unsubstituted.

78. The compound of any one of claims 70-77, wherein each R^{e} and R^{f} is independently selected from R¹¹ and hydrogen.

79. The compound of claim 78, wherein at least one of R^{e} and R^{f} is —OR¹² or C₁₋₆alkyl that is unsubstituted or substituted with one or more R²⁰ (e.g., C₁₋₆ alkyl that is unsubstituted or substituted with —OH or —CN).

80. The compound of claim 78, wherein each R^{e} and R^{f} is hydrogen.

81. The compound of claim 78, wherein 0-2 R^{e} and R^{f} groups are C₁₋₆ alkyl, and the remaining R^{e} and R^{f} groups are H.

82. The compound of any one of claims 70-76, wherein each R^{e}, R^{f}, and R^{g} is H.

83. The compound of any one of claims 70-77, wherein an R^{e} and an R^{f} join together to form a 4-6 membered ring.

84. The compound of any one of claims 70-77, wherein a first R^{f} and a second R^{f} connected to adjacent atoms join together to form a 3-5 membered ring, or a first R^{e} and a second R^{f} connected to adjacent atoms join together to form a 3-5 membered ring.

85. The compound of any one of claims 1-12 and 45-84, wherein the compound is a compound according to Formula IG, IH, IJ, IK IL, IM, or IN: 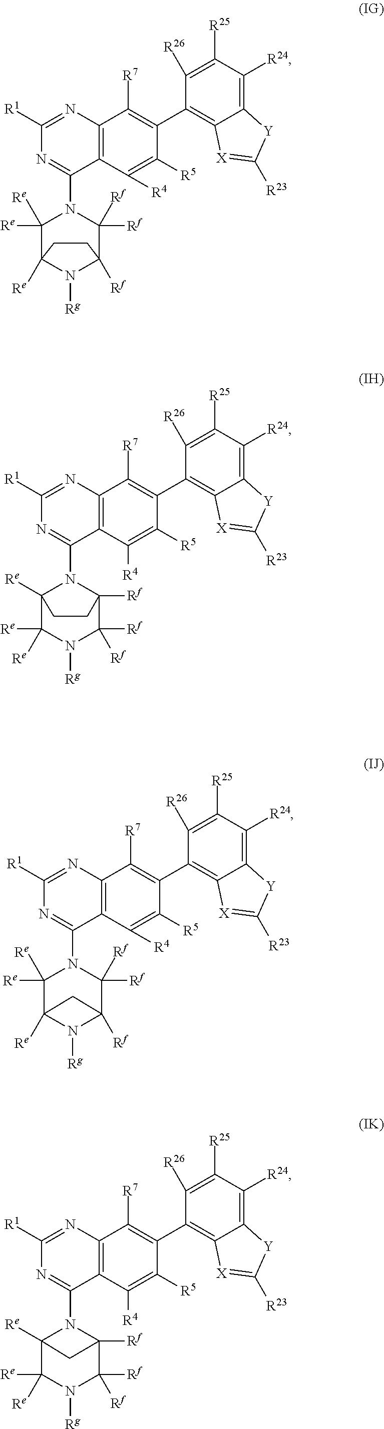 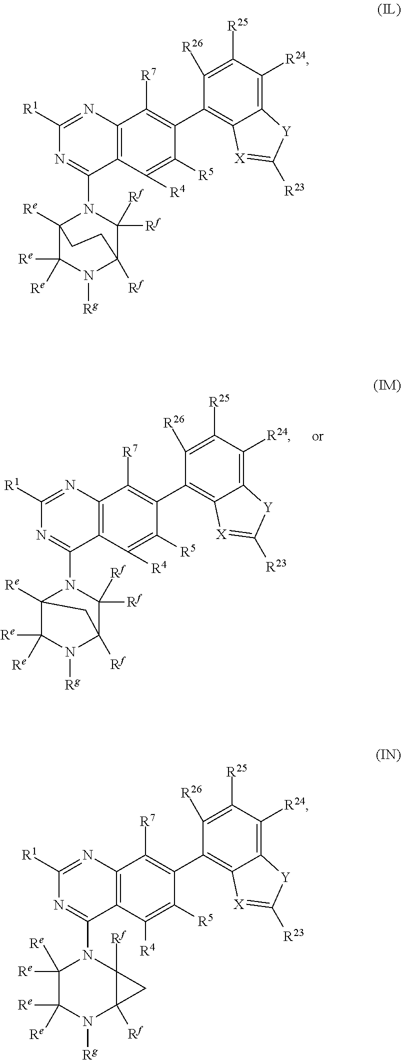
or a salt (e.g., pharmaceutically acceptable salt) thereof, wherein:
each R^{e} and R^{f} is independently selected from R¹¹ and hydrogen;
R^{g} is selected from H and C₁₋₆alkyl, wherein any C₁₋₆alkyl is unsubstituted or substituted with one or more R²⁰;
X is selected from N and C—CN;
Y is selected from O and S;
R²³ is selected from —N(R¹²)₂, C₁₋₆alkyl, and C₁₋₆alkyl-N(R¹⁴)₂, wherein any C₁₋₆alkyl is unsubstituted or substituted with one or more R¹³; and
R²⁴, R²⁵, and R²⁶ are independently selected from H, halogen, and C₁₋₆alkyl, wherein any C₁₋₆alkyl is unsubstituted or substituted with one or more R¹³.

86. The compound of claim 85, wherein the compound is a compound of Formula IG or a salt (e.g., a pharmaceutically acceptable salt) thereof.

87. The compound of claim 85, wherein the compound is a compound of Formula IH or a salt (e.g., a pharmaceutically acceptable salt) thereof.

88. The compound of claim 85, wherein the compound is a compound of Formula IJ or a salt (e.g., a pharmaceutically acceptable salt) thereof.

89. The compound of claim 85, wherein the compound is a compound of Formula IK or a salt (e.g., a pharmaceutically acceptable salt) thereof.

90. The compound of claim 85, wherein the compound is a compound of Formula IL or a salt (e.g., a pharmaceutically acceptable salt) thereof.

91. The compound of claim 85, wherein the compound is a compound of Formula IM or a salt (e.g., a pharmaceutically acceptable salt) thereof.

92. The compound of claim 85, wherein the compound is a compound of Formula IN or a salt (e.g., a pharmaceutically acceptable salt) thereof.

93. The compound of any one of claims 85-92, wherein X is C—CN and Y is S.

94. The compound of any one of claims 85-93, wherein R²³ is —N(R¹²)₂.

95. The compound of any one of claims 85-94, wherein X is C—CN, Y is S, R²³ is —N(R²)₂, and R²⁴ is a halogen (e.g., F).

96. The compound of any one of claims 85-95, wherein 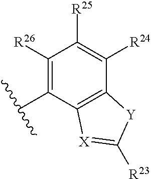
is

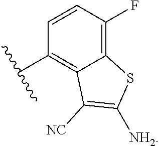 [SMILES: *c1ccc(F)c2sc(N)c(C#N)c12]

97. The compound of any one of claims 85-96, wherein R^{g} is selected from H and C₁₋₆alkyl that is unsubstituted.

98. The compound of claim 97, wherein R^{g} is H.

99. The compound of claim 97, wherein R^{g} is C₁₋₆alkyl that is unsubstituted.

100. The compound of any one of claims 85-99, wherein at least one of R^{e} and R^{f} is —OR² or C₁₋₆alkyl that is unsubstituted or substituted with one or more R²⁰ (e.g., C₁₋₆ alkyl that is unsubstituted or substituted with —OH or —CN).

101. The compound of any one of claims 85-100, wherein each R^{e} and R^{f} is hydrogen.

102. The compound of any one of claims 85-100, wherein 0-2 R^{e} and R^{f} groups are C₁₋₆ alkyl and the remaining R^{e} and R^{f} groups are H.

103. The compound of any one of claims 85-99, wherein each R^{e}, R^{f}, and R^{g} is H.

104. The compound of any one of claims 1-103, wherein R⁴ is H.

105. The compound of any one of claims 1-103, wherein R⁴ is —OR¹⁴.

106. The compound of claim 105, wherein R⁴ is —OCH₃.

107. The compound of any one of claims 1-106, wherein R⁵ is a halogen (e.g., F or Cl).

108. The compound of any one of claims 1-106, wherein R⁵ is selected from C₁₋₆alkyl that is unsubstituted or substituted with one or more R¹.

109. The compound of claim 108, wherein RS is selected from C₁₋₆alkyl that is substituted with one or more halogens or —CN.

110. The compound of claim 109, wherein R⁵ is selected from —CF₂H, —CF₃, —CH₂CN, and —CH₂CH₃.

111. The compound of claim 110, wherein R^{S} is —CF₃.

112. The compound of any one of claims 1-106, wherein R⁵ is —CN or —OR¹².

113. The compound of any one of claims 1-106, wherein R⁵ is a 3-6 membered heterocycle.

114. The compound of any one of claims 1-106, wherein R⁵ is a 5-6 membered heteroaryl.

115. The compound of any one of claims 1-114, wherein R⁷ is F.

116. The compound of any one of claims 1-115, wherein R¹ is H.

117. The compound of any one of claims 1-115, wherein R¹ is C₁₋₆alkyl that is unsubstituted or substituted with one or more R¹³.

118. The compound of claim 117, wherein R¹ is C₁₋₃alkyl that is unsubstituted or substituted with one or more R¹³.

119. The compound of claim 118, wherein R¹ is C₁₋₃alkyl that is unsubstituted.

120. The compound of any one of claims 1-115, wherein R¹ is —OR⁸ or —O(C₁₋₆alkylene)R⁸, wherein any C₁₋₆alkylene is unsubstituted or substituted with one or more R¹³.

121. The compound of claim 120, wherein R¹ is —OR⁸.

122. The compound of claim 120, wherein R¹ is —O(C₁₋₆alkylene)R⁸.

123. The compound of claim 122, wherein R¹ is —O(C₁₋₆alkylene)R, wherein any C₁₋₆alkylene is unsubstituted.

124. The compound of any one of claims 120-123, wherein R⁸ is H.

125. The compound of any one of claims 120-123, wherein R⁸ is C₁₋₆alkyl that is unsubstituted or substituted with one or more R¹³.

126. The compound of claim 125, wherein R⁸ is C₁₋₃alkyl that is unsubstituted or substituted with one or more R¹³.

127. The compound of claim 126, wherein R⁸ is C₁₋₃alkyl that is unsubstituted.

128. The compound of any one of claims 125-127, wherein R¹ is selected from

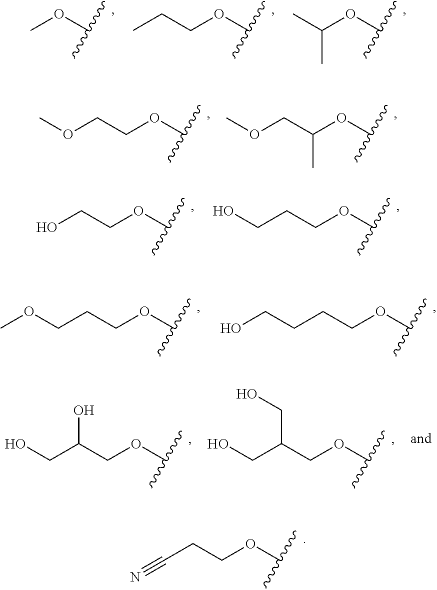

129. The compound of any one of claims 120-123, wherein R⁸ is a heteroaryl that comprises 3-8 members and is unsubstituted or substituted with one or more R¹⁹.

130. The compound of claim 129, wherein R⁸ is a heteroaryl that comprises 5-8 members including one or more heteroatoms selected from N, O, and S.

131. The compound of claim 130, wherein R⁸ is a heteroaryl that comprises 5-8 members including two or more heteroatoms selected from N, O, and S.

132. The compound of claim 131, wherein R⁸ is a heteroaryl that comprises 5-8 members including two nitrogen atoms.

133. The compound of any one of claims 129-132, wherein R⁸ is a heteroaryl that is unsubstituted.

134. The compound of any one of claims 129-133, wherein R¹ is

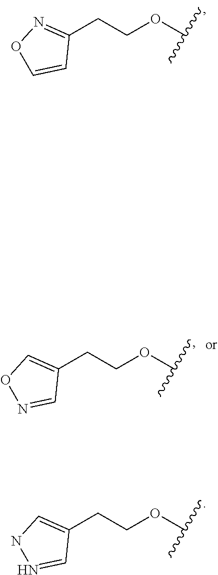 [SMILES: *OCCc1ccon1 ; *OCCc1cnoc1 ; *OCCc1cn[nH]c1]

135. The compound of any one of claims 120-123, wherein R⁸ is a heterocycle that comprises 3-8 members and is unsubstituted or substituted with one or more R¹⁹.

136. The compound of claim 135, wherein R⁸ is a heterocycle that comprises 3-8 members including one or more heteroatoms selected from N, O, and S.

137. The compound of claim 136, wherein R⁸ is a heterocycle that comprises 3-8 members including two or more heteroatoms selected from N, O, and S.

138. The compound of claim 137, wherein R⁸ is a heterocycle that comprises 3-8 members including three or more heteroatoms selected from N, O, and S.

139. The compound of claim 137, wherein R⁸ is a heterocycle that comprises 3-8 members including two oxygen atoms.

140. The compound of any one of claims 135-139, wherein R⁸ is a heterocycle that is unsubstituted.

141. The compound of any one of claims 135-140, wherein R¹ is selected from

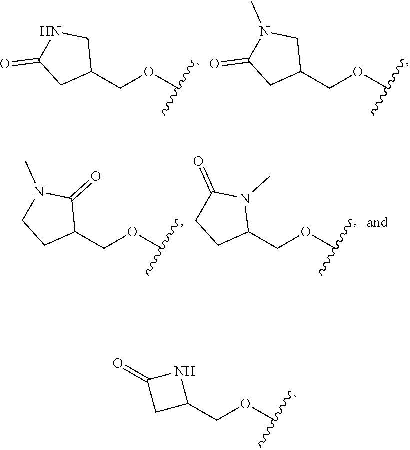

142. The compound of any one of claims 135-140, wherein R¹ is selected from

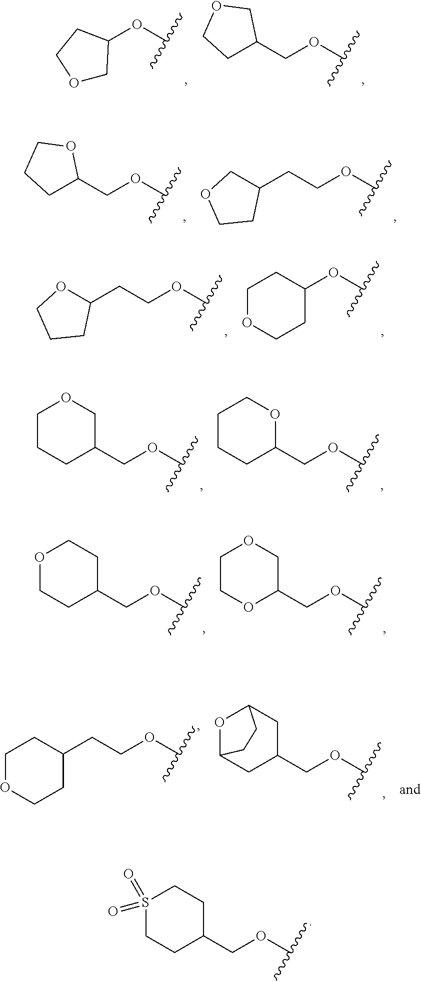

143. The compound of any one of claims 135-140, wherein R¹ is not a structure selected from:
(i) 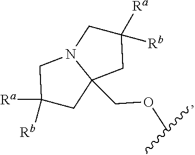
wherein R^{a} and R^{b} are each independently selected from deuterium, halogen, C₁₋₆alkyl, —OR¹², and H, wherein any C₁₋₆alkyl is unsubstituted or is substituted with one or more R¹³;
(ii) 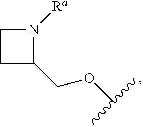
wherein R^{a} is selected from halogen, C₁₋₆alkyl, —OR¹², and H, wherein any C₁₋₆alkyl is unsubstituted or is substituted with one or more R¹³;
(iii) 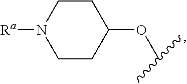
wherein R^{a} is selected from C₁₋₆alkyl and H, wherein any C₁₋₆alkyl is unsubstituted or is substituted with one or more R¹³; and
(iv) 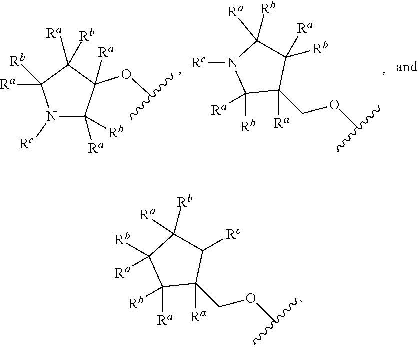
wherein each R^{a} and R^{b} is independently selected from halogen, C₁₋₆ alkyl, —OR¹², and H; and R^{c} is selected from C₁₋₆ alkyl, wherein the C₁₋₆ alkyl is unsubstituted or is substituted with one or more R¹³, or an R^{a} and R^{b} on the same atom combine to form a 3-membered carbocycle.

144. The compound of any one of claims 120-123, wherein R⁸ is phenyl that is unsubstituted or substituted with one or more R¹⁹.

145. The compound of claim 144, wherein R⁸ is phenyl that is unsubstituted.

146. The compound of any one of claims 144-145, wherein R¹ is

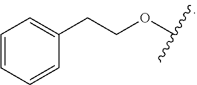 [SMILES: *OCCc1ccccc1]

147. The compound of any one of claims 120-123, wherein R⁸ is a carbocycle that comprises 3-8 members and is unsubstituted or substituted with one or more R¹⁹.

148. The compound of claim 147, wherein R⁸ is a carbocycle that comprises 3-6 members.

149. The compound of claim 147 or 148, wherein R⁸ is a carbocycle that is unsubstituted.

150. The compound of any one of claims 147-149, wherein R¹ is selected from

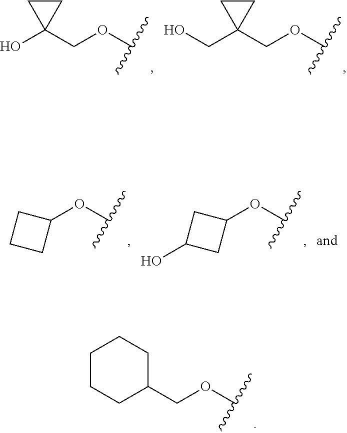 [SMILES: *OCC1(O)CC1 ; *OCC1(CO)CC1 ; *OC1CCC1 ; *OC1CC(O)C1 ; *OCC1CCCCC1]

151. The compound of any one of claims 1-115, wherein R¹ is —N(R¹⁶)₂.

152. The compound of claim 151, wherein R¹ is —NHR¹⁶.

153. The compound of claim 151 or 152, wherein each R¹⁶ is independently selected from C₁₋₆alkyl that is unsubstituted or substituted with one or more R¹³.

154. The compound of claim 151 or 152, wherein each R¹⁶ is independently selected from C₁₋₆alkyl and —C(O)C₁₋₆alkyl, wherein any C₁₋₆alkyl is unsubstituted or substituted with one or more R¹³.

155. The compound of any one of claims 151-154, wherein R¹ is selected from

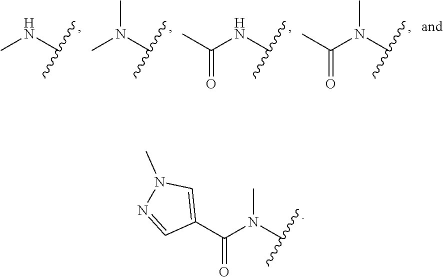

156. The compound of any one of claims 1-115, wherein R¹ is a 4-7 membered nitrogen-containing heterocycle that is unsubstituted or substituted with one or more R¹⁷.

157. The compound of claim 156, wherein R¹ is a 4-5 membered nitrogen-containing heterocycle.

158. The compound of claim 156 or 157, wherein R¹ is a nitrogen-containing heterocycle that is unsubstituted.

159. The compound of any one of claims 156-158, wherein R¹ is selected from

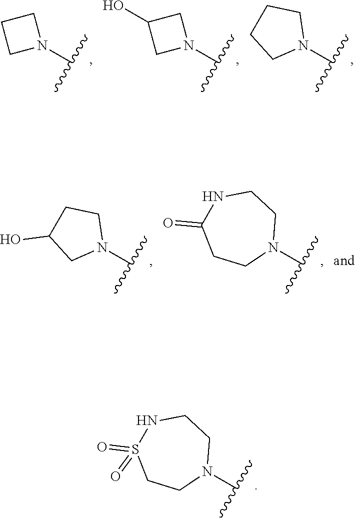

160. The compound of any one of claims 156-159, wherein R¹ is not

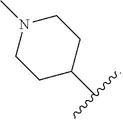 [SMILES: *C1CCN(C)CC1]

161. A compound shown in Table 2, or a salt (e.g., pharmaceutically acceptable salt) thereof.

162. A compound shown in Table 3, or a salt (e.g., pharmaceutically acceptable salt) thereof.

163. A pharmaceutical composition comprising a compound of any one of claims 1-162, or a salt (e.g., pharmaceutically acceptable salt) thereof, and a pharmaceutically acceptable excipient.

164. A compound of any one of claims 1-162, or a salt (e.g., pharmaceutically acceptable salt) thereof, for use as a medicament.

165. The compound of claim 164, wherein the medicament is useful in the prevention or treatment of a disease, disorder, or condition ameliorated by the inhibition of KRAS having a G12D mutation.

166. The compound of claim 164 or 165, wherein the medicament is useful in the prevention or treatment of a cancer.

167. The compound of claim 166, wherein the cancer is selected from the group consisting of pancreatic cancer, colorectal cancer, and lung cancer.

168. A compound of any one of claims 1-162, or a salt (e.g., pharmaceutically acceptable salt) thereof, for use in the treatment of a disease, disorder, or condition.

169. The compound of claim 168, wherein the disease, disorder, or condition is a cancer.

170. The compound of claim 169, wherein the cancer is selected from the group consisting of pancreatic cancer, colorectal cancer, and lung cancer.

171. The compound of any one of claims 168-170, wherein the compound is used in the treatment of a disease, disorder, or condition in a subject in need thereof.

172. A compound of any one of claims 1-162, or a salt (e.g., pharmaceutically acceptable salt) thereof, for use in the manufacture of a medicament.

173. The compound of claim 172, wherein the medicament is useful in the prevention or treatment of a disease, disorder, or condition ameliorated by the inhibition of KRAS having a G12D mutation.

174. The compound of claim 172 or 173, wherein the medicament is useful in the treatment of a cancer.

175. The compound of claim 174, wherein the cancer is selected from the group consisting of pancreatic cancer, colorectal cancer, and lung cancer.

176. A method, comprising administering a therapeutically effective amount of a compound of any one of claims 1-162, or a salt (e.g., pharmaceutically acceptable salt) thereof, to a subject in need thereof.

177. The method of claim 176, wherein the subject has a disease, disorder, or condition ameliorated by the inhibition of KRAS having a G12D mutation.

178. The method of claim 176 or 177, wherein the subject has a cancer.

179. The method of claim 178, wherein the subject was previously diagnosed with the cancer.

180. The method of claim 178, wherein the subject has previously undergone a treatment regimen for the cancer.

181. The method of claim 180, wherein the subject has previously entered remission from the cancer.

182. The method of any one of claims 178-181, wherein the cancer is selected from the group consisting of pancreatic cancer, colorectal cancer, and lung cancer.

183. The method of any one of claims 176-182, wherein the compound, or the salt thereof, is administered in combination with an additional therapeutic agent.

184. The use of a compound of any one of claims 1-162, or a salt (e.g., pharmaceutically acceptable salt) thereof, for the manufacture of a medicament for the treatment of a cancer.

185. The use of claim 184, wherein the cancer is selected from the group consisting of pancreatic cancer, colorectal cancer, and lung cancer.

186. A method, comprising contacting a KRAS protein with a compound of any one of claims 1-162, or a salt (e.g., pharmaceutically acceptable salt) thereof.

187. The method of claim 186, wherein contacting the KRAS protein with the compound modulates KRAS.

188. The method of claim 186 or 187, wherein the KRAS protein has a G12D mutation.

189. The method of any one of claims 186-188, wherein the KRAS protein is in an active (GTP-bound) state.

190. The method of any one of claims 186-188, wherein the KRAS protein is in an inactive (GDP-bound) state.

191. The method of any one of claims 186-190, wherein the KRAS protein is located within a cell.

192. The method of claim 191, wherein the cell is located within a subject.

193. The method of claim 192, wherein the subject is a human.

194. The method of claim 192 or 193, wherein the subject has a cancer.

195. The method of claim 194, wherein the cancer is selected from the group consisting of pancreatic cancer, colorectal cancer, and lung cancer.

196. A method of inhibiting the function of a KRAS protein having a G12D mutation, comprising contacting the KRAS protein with a compound of any one of claims 1-162, or a salt (e.g., pharmaceutically acceptable salt) thereof.

197. The method of claim 196, wherein the KRAS protein has a G12D mutation.

198. The method of claim 196 or 197, wherein the KRAS protein is in an active (GTP-bound) state.

199. The method of claim 196 or 197, wherein the KRAS protein is in an inactive (GDP-bound) state.

200. The method of any one of claims 196-199, wherein the KRAS protein is located within a cell.

201. The method of claim 200, wherein the cell is located within a subject.

202. The method of claim 201, wherein the subject is a human.

203. The method of claim 201 or 202, wherein the subject has a cancer.

204. The method of claim 203, wherein the cancer is selected from the group consisting of pancreatic cancer, colorectal cancer, and lung cancer.

205. A compound capable of inhibiting a KRAS protein having a G12D mutation in both its active (GTP-bound) and inactive (GDP-bound) state.

206. The compound of claim 205, wherein the compound:
(i) has IC₅₀≤0.1 μM, 0.1 μM<IC₅₀<1 μM, or 1 μM<IC₅₀≤10 μM in the assay of Biological Example 1 (e.g., a protein:protein interaction (PPI) Homogenous Time Resolved Fluorescence (HTRF) analysis of 50 nM Avi-KRAS G12D (amino acids 1-169) GppNHp/RAF1 RBD-3×FLAG (52-151); 50 nM Avi-KRAS G12R (amino acids 1-169) GppNHp/RAF1 RBD-3×FLAG (52-151); 50 nM Avi-KRAS G12V (amino acids 1-169) GppNHp/RAF1 RBD-3×FLAG (52-151); 50 nM Avi-KRAS WT (amino acids 1-169) GppNHp/RAF1 RBD-3×FLAG (52-151); and/or 75 nM Avi-RAF1 RBD-3×FLAG); and/or
(ii) has IC₅₀≤0.1 μM or 0.1 μM<IC₅₀≤1 μM in the assay of Biological Example 2 (e.g., cell-based pERK HTRF assay in GP2d (G12D) cell; and/or
(iii) has IC₅₀≤0.1 μM, 0.1 μM<IC₅₀≤1 μM, 1 μM<IC₅₀≤10 μM, or IC₅₀>10 μM in the assay of Biological Example 3 (e.g., cell-based pERK HTRF assay in SW620 (G12V) cell).

207. The compound of claim 206, wherein the compound:
(i) has IC₅₀≤0.1 μM or 0.1 μM<IC₅₀≤1 μM in the assay of Biological Example 1; and/or
(ii) has IC₅₀≤0.1 M or 0.1 μM<IC₅₀≤1 μM in the assay of Biological Example 2.

208. The compound of any one of claims 205-207, wherein the compound is capable of irreversibly binding the KRAS protein.

209. The compound of any one of claims 205-207, wherein the compound is capable of reversibly binding the KRAS protein.

210. The compound of any one of claims 205-209, wherein the compound is a compound according to any one of claims 1-162.
